SEQUENCE LISTING (33 sequences)

SEQ ID NO: 1 - DNA, 1804 bp, Homo sapiens
gtgcagcccgtgccccccgcgcgccggggccgaatgcgcgccgcgtagggtcccccgggccgagaggggtgcccggagggaagagcgcggtgggggcgccccggccccgctgccctggggctatggccatggtgaccggcggctggggcggccccggcggcgacacgaacggcgtggacaaggcgggcggctacccgcgcgcggccgaggacgactcggcctcgccccccggtgccgccagcgacgccgagccgggcgacgaggagcggccggggctgcaggtggactgcgtggtgtgcggggacaagtcgagcggcaagcattacggtgtcttcacctgcgagggctgcaagagctttttcaagcgaagcatccgccgcaacctcagctacacctgccggtccaaccgtgactgccagatcgaccagcaccaccggaaccagtgccagtactgccgtctcaagaagtgcttccgggtgggcatgaggaaggaggcggtgcagcgcggccgcatcccgcactcgctgcctggtgccgtggccgcctcctcgggcagccccccgggctcggcgctggcggcagtggcgagcggcggagacctcttcccggggcagccggtgtccgaactgatcgcgcagctgctgcgcgctgagccctaccctgcggcggccggacgcttcggcgcagggggcggcgcggcgggcgcggtgctgggcatcgacaacgtgtgcgagctggcggcgcggctgctcttcagcaccgtggagtgggcgcgccacgcgcccttcttccccgagctgccggtggccgaccaggtggcgctgctgcgcctgagctggagcgagctcttcgtgctgaacgcggcgcaggcggcgctgcccctgcacacggcgccgctactggccgccgccggcctccacgccgcgcctatggccgccgagcgcgccgtggctttcatggaccaggtgcgcgccttccaggagcaggtggacaagctgggccgcctgcaggtcgactcggccgagtatggctgcctcaaggccatcgcgctcttcacgcccgacgcctgtggcctctcagacccggcccacgttgagagcctgcaggagaaggcgcaggtggccctcaccgagtatgtgcgggcgcagtacccgtcccagccccagcgcttcgggcgcctgctgctgcggctccccgccctgcgcgcggtccctgcctccctcatctcccagctgttcttcatgcgcctggtggggaagacgcccattgagacactgatcagagacatgctgctgtcggggagtaccttcaactggccctacggctcgggccagtgaccatgacggggccacgtgtgctgtggccaggcctgcagacagacctcaagggacagggaatgctgaggcctcgaggggcctcccggggcccaggactctggcttctctcctcagacttctattttttaaagactgtgaaatgtttgtcttttctgttttttaaatgatcatgaaaccaaaaagagactgatcatccaggcctcagcctcatcctccccaggacccctgtccaggatggagggtccaatcctaggacagccttgttcctcagcacccctagcatgaacttgtgggatggtggggttggcttccctggcatgatggacaaaggcctggcgtcggccagaggggctgctccagtgggcaggggtagctagcgtgtgccaggcagatcctctggacacgtaacctatgtcagacactacatgatgactcaaggccaataataaagacatttcctacctgca

SEQ ID NO: 2 - protein, 404 aa, Homo sapiens
MAMVTGGWGGPGGDTNGVDKAGGYPRAAEDDSASPPGAASDAEPGDEERPGLQVDCVVCGDKSSGKHYGVFTCEGCKSFFKRSIRRNLSYTCRSNRDCQIDQHHRNQCQYCRLKKCFRVGMRKEAVQRGRIPHSLPGAVAASSGSPPGSALAAVASGGDLFPGQPVSELIAQLLRAEPYPAAAGRFGAGGGAAGAVLGIDNVCELAARLLFSTVEWARHAPFFPELPVADQVALLRLSWSELFVLNAAQAALPLHTAPLLAAAGLHAAPMAAERAVAFMDQVRAFQEQVDKLGRLQVDSAEYGCLKAIALFTPDACGLSDPAHVESLQEKAQVALTEYVRAQYPSQPQRFGRLLLRLPALRAVPASLISQLFFMRLVGKTPIETLIRDMLLSGSTFNWPYGSGQ

SEQ ID NO: 3 - DNA, 1061 bp, Homo sapiens
cggctgctcttcagcaccgtggagtgggcgcgccacgcgcccttcttccccgagctgccggtggccgaccaggtggcgctgctgcgcctgagctggagcgagctcttcgtgctgaacgcggcgcaggcggcgctgcccctgcacacggcgccgctactggccgccgccggcctccacgccgcgcctatggccgccgagcgcgccgtggctttcatggaccaggtgcgcgccttccaggagcaggtggacaagctgggccgcctgcaggtcgactcggccgagtatggctgcctcaaggccatcgcgctcttcacgcccgacgcctgtggcctctcagacccggcccacgttgagagcctgcaggagaaggcgcaggtggccctcaccgagtatgtgcgggcgcagtacccgtcccagccccagcgcttcgggcgcctgctgctgcggctccccgccctgcgcgcggtccctgcctccctcatctcccagctgttcttcatgcgcctggtggggaagacgcccattgagacactgatcagagacatgctgctgtcggggagtaccttcaactggccctacggctcgggccagtgaccatgacggggccacgtgtgctgtggccaggcctgcagacagacctcaagggacagggaatgctgaggcctcgaggggcctcccggggcccaggactctggcttctctcctcagacttctattttttaaagactgtgaaatgtttgtcttttctgttttttaaatgatcatgaaaccaaaaagagactgatcatccaggcctcagcctcatcctccccaggacccctgtccaggatggagggtccaatcctaggacagccttgttcctcagcacccctagcatgaacttgtgggatggtggggttggcttccctggcatgatggacaaaggcctggcgtcggccagaggggctgctccagtgggcaggggtagctagcgtgtgccaggcagatcctctggacacgtaacctatgtcagacactacatgatgactcaaggccaataataaagacatttcctacctgca

SEQ ID NO: 4 - protein, 198 aa, Homo sapiens
ARLLFSTVEWARHAPFFPELPVADQVALLRLSWSELFVLNAAQAALPLHTAPLLAAAGLHAAPMAAERAVAFMDQVRAFQEQVDKLGRLQVDSAEYGCLKAIALFTPDACGLSDPAHVESLQEKAQVALTEYVRAQYPSQPQRFGRLLLRLPALRAVPASLISQLFFMRLVGKTPIETLIRDMLLSGSTFNWPYGSGQ

SEQ ID NO: 5 - DNA, 1804 bp, Homo Sapiens
  mutation (369)..(370): S83A mutant of NR2F6
gtgcagcccgtgccccccgcgcgccggggccgaatgcgcgccgcgtagggtcccccgggccgagaggggtgcccggagggaagagcgcggtgggggcgccccggccccgctgccctggggctatggccatggtgaccggcggctggggcggccccggcggcgacacgaacggcgtggacaaggcgggcggctacccgcgcgcggccgaggacgactcggcctcgccccccggtgccgccagcgacgccgagccgggcgacgaggagcggccggggctgcaggtggactgcgtggtgtgcggggacaagtcgagcggcaagcattacggtgtcttcacctgcgagggctgcaagagctttttcaagcgagccatccgccgcaacctcagctacacctgccggtccaaccgtgactgccagatcgaccagcaccaccggaaccagtgccagtactgccgtctcaagaagtgcttccgggtgggcatgaggaaggaggcggtgcagcgcggccgcatcccgcactcgctgcctggtgccgtggccgcctcctcgggcagccccccgggctcggcgctggcggcagtggcgagcggcggagacctcttcccggggcagccggtgtccgaactgatcgcgcagctgctgcgcgctgagccctaccctgcggcggccggacgcttcggcgcagggggcggcgcggcgggcgcggtgctgggcatcgacaacgtgtgcgagctggcggcgcggctgctcttcagcaccgtggagtgggcgcgccacgcgcccttcttccccgagctgccggtggccgaccaggtggcgctgctgcgcctgagctggagcgagctcttcgtgctgaacgcggcgcaggcggcgctgcccctgcacacggcgccgctactggccgccgccggcctccacgccgcgcctatggccgccgagcgcgccgtggctttcatggaccaggtgcgcgccttccaggagcaggtggacaagctgggccgcctgcaggtcgactcggccgagtatggctgcctcaaggccatcgcgctcttcacgcccgacgcctgtggcctctcagacccggcccacgttgagagcctgcaggagaaggcgcaggtggccctcaccgagtatgtgcgggcgcagtacccgtcccagccccagcgcttcgggcgcctgctgctgcggctccccgccctgcgcgcggtccctgcctccctcatctcccagctgttcttcatgcgcctggtggggaagacgcccattgagacactgatcagagacatgctgctgtcggggagtaccttcaactggccctacggctcgggccagtgaccatgacggggccacgtgtgctgtggccaggcctgcagacagacctcaagggacagggaatgctgaggcctcgaggggcctcccggggcccaggactctggcttctctcctcagacttctattttttaaagactgtgaaatgtttgtcttttctgttttttaaatgatcatgaaaccaaaaagagactgatcatccaggcctcagcctcatcctccccaggacccctgtccaggatggagggtccaatcctaggacagccttgttcctcagcacccctagcatgaacttgtgggatggtggggttggcttccctggcatgatggacaaaggcctggcgtcggccagaggggctgctccagtgggcaggggtagctagcgtgtgccaggcagatcctctggacacgtaacctatgtcagacactacatgatgactcaaggccaataataaagacatttcctacctgca

SEQ ID NO: 6 - protein, 404 aa, Homo sapiens
  VARIANT (83)..(83): S83A mutation
MAMVTGGWGGPGGDTNGVDKAGGYPRAAEDDSASPPGAASDAEPGDEERPGLQVDCVVCGDKSSGKHYGVFTCEGCKSFFKRAIRRNLSYTCRSNRDCQIDQHHRNQCQYCRLKKCFRVGMRKEAVQRGRIPHSLPGAVAASSGSPPGSALAAVASGGDLFPGQPVSELIAQLLRAEPYPAAAGRFGAGGGAAGAVLGIDNVCELAARLLFSTVEWARHAPFFPELPVADQVALLRLSWSELFVLNAAQAALPLHTAPLLAAAGLHAAPMAAERAVAFMDQVRAFQEQVDKLGRLQVDSAEYGCLKAIALFTPDACGLSDPAHVESLQEKAQVALTEYVRAQYPSQPQRFGRLLLRLPALRAVPASLISQLFFMRLVGKTPIETLIRDMLLSGSTFNWPYGSGQ

SEQ ID NO: 7 - DNA, 1804 bp, Homo sapiens
  mutation (369)..(371): S83E mutant of NR2F6
gtgcagcccgtgccccccgcgcgccggggccgaatgcgcgccgcgtagggtcccccgggccgagaggggtgcccggagggaagagcgcggtgggggcgccccggccccgctgccctggggctatggccatggtgaccggcggctggggcggccccggcggcgacacgaacggcgtggacaaggcgggcggctacccgcgcgcggccgaggacgactcggcctcgccccccggtgccgccagcgacgccgagccgggcgacgaggagcggccggggctgcaggtggactgcgtggtgtgcggggacaagtcgagcggcaagcattacggtgtcttcacctgcgagggctgcaagagctttttcaagcgagagatccgccgcaacctcagctacacctgccggtccaaccgtgactgccagatcgaccagcaccaccggaaccagtgccagtactgccgtctcaagaagtgcttccgggtgggcatgaggaaggaggcggtgcagcgcggccgcatcccgcactcgctgcctggtgccgtggccgcctcctcgggcagccccccgggctcggcgctggcggcagtggcgagcggcggagacctcttcccggggcagccggtgtccgaactgatcgcgcagctgctgcgcgctgagccctaccctgcggcggccggacgcttcggcgcagggggcggcgcggcgggcgcggtgctgggcatcgacaacgtgtgcgagctggcggcgcggctgctcttcagcaccgtggagtgggcgcgccacgcgcccttcttccccgagctgccggtggccgaccaggtggcgctgctgcgcctgagctggagcgagctcttcgtgctgaacgcggcgcaggcggcgctgcccctgcacacggcgccgctactggccgccgccggcctccacgccgcgcctatggccgccgagcgcgccgtggctttcatggaccaggtgcgcgccttccaggagcaggtggacaagctgggccgcctgcaggtcgactcggccgagtatggctgcctcaaggccatcgcgctcttcacgcccgacgcctgtggcctctcagacccggcccacgttgagagcctgcaggagaaggcgcaggtggccctcaccgagtatgtgcgggcgcagtacccgtcccagccccagcgcttcgggcgcctgctgctgcggctccccgccctgcgcgcggtccctgcctccctcatctcccagctgttcttcatgcgcctggtggggaagacgcccattgagacactgatcagagacatgctgctgtcggggagtaccttcaactggccctacggctcgggccagtgaccatgacggggccacgtgtgctgtggccaggcctgcagacagacctcaagggacagggaatgctgaggcctcgaggggcctcccggggcccaggactctggcttctctcctcagacttctattttttaaagactgtgaaatgtttgtcttttctgttttttaaatgatcatgaaaccaaaaagagactgatcatccaggcctcagcctcatcctccccaggacccctgtccaggatggagggtccaatcctaggacagccttgttcctcagcacccctagcatgaacttgtgggatggtggggttggcttccctggcatgatggacaaaggcctggcgtcggccagaggggctgctccagtgggcaggggtagctagcgtgtgccaggcagatcctctggacacgtaacctatgtcagacactacatgatgactcaaggccaataataaagacatttcctacctgca

SEQ ID NO: 8 - protein, 404 aa, Homo sapiens
  VARIANT (83)..(83): S83E mutation
MAMVTGGWGGPGGDTNGVDKAGGYPRAAEDDSASPPGAASDAEPGDEERPGLQVDCVVCGDKSSGKHYGVFTCEGCKSFFKREIRRNLSYTCRSNRDCQIDQHHRNQCQYCRLKKCFRVGMRKEAVQRGRIPHSLPGAVAASSGSPPGSALAAVASGGDLFPGQPVSELIAQLLRAEPYPAAAGRFGAGGGAAGAVLGIDNVCELAARLLFSTVEWARHAPFFPELPVADQVALLRLSWSELFVLNAAQAALPLHTAPLLAAAGLHAAPMAAERAVAFMDQVRAFQEQVDKLGRLQVDSAEYGCLKAIALFTPDACGLSDPAHVESLQEKAQVALTEYVRAQYPSQPQRFGRLLLRLPALRAVPASLISQLFFMRLVGKTPIETLIRDMLLSGSTFNWPYGSGQ

SEQ ID NO: 9 - DNA, 1590 bp, artificial sequence
  /note=“Description of artificial sequence coding sequence of NR2F6 wild-type Er-LBD fusion mutant”
atggccatggtgaccggcggctggggcggccccggcggcgacacgaacggcgtggacaaggcgggcggctacccgcgcgcggccgaggacgactcggcctcgccccccggtgccgccagcgacgccgagccgggcgacgaggagcggccggggctgcaggtggactgcgtggtgtgcggggacaagtcgagcggcaagcattacggtgtcttcacctgcgagggctgcaagagctttttcaagcgaagcatccgccgcaacctcagctacacctgccggtccaaccgtgactgccagatcgaccagcaccaccggaaccagtgccagtactgccgtctcaagaagtgcttccgggtgggcatgaggaaggaggcggtgcagcgcggccgcatcccgcactcgctgcctggtgccgtggccgcctcctcgggcagccccccgggctcggcgctggcggcagtggcgagcggcggagacctcttcccggggcagccggtgtccgaactgatcgcgcagctgctgcgcgctgagccctaccctgcggcggccggacgcttcggcgcagggggcggcgcggcgggcgcggtgctgggcatcgacaacgtgtgcgagctggcggcgtgggatccacgaaatgaaatgggtgcttcaggagacatgagggctgccaacctttggccaagccctcttgtgattaagcacactaagaagaatagccctgccttgtccttgacagctgaccagatggtcagtgccttgttggatgctgaaccgcccatgatctattctgaatatgatccttctagacccttcagtgaagcctcaatgatgggcttattgaccaacctagcagatagggagctggttcatatgatcaactgggcaaagagagtgccaggctttggggacttgaatctccatgatcaggtccaccttctcgagtgtgcctggctggagattctgatgattggtctcgtctggcgctccatggaacacccggggaagctcctgtttgctcctaacttgctcctggacaggaatcaaggtaaatgtgtggaaggcatggtggagatctttgacatgttgctggctacgtcaagtcggttccgcatgatgaacctgcagggagaagagtttgtgtgcctcaaatccatcattttgcttaattccggagtgtacacgtttctgtccagcaccttgaagtctctggaagagaaggaccacatccaccgtgtcctggacaagatcacagacactttgatccacctgatggccaaagctggcctgactctgcagcagcagcatcgccgcctagctcagctccttctcattctttcccatatccggcacatgagtaacaaaggcatggagcatctctacaacatgaaatgcaagaacgttgtgcccctctatgacctgctcctggagatgttggatgcccaccgccttcatgccccagccagtcgcatgggagtgcccccagaggagcccagccagacccagctggccaccaccagctccacttcagcacattccttacaaacctactacatacccccggaagcagagggcttccccaacacgatctga

SEQ ID NO: 10 - protein, 528 aa, artificial sequence
  /note=“Description of artificial sequence amino acid sequence of NR2F6 wild-type Er-LBD fusion mutant”
MAMVTGGWGGPGGDTNGVDKAGGYPRAAEDDSASPPGAASDAEPGDEERPGLQVDCVVCGDKSSGKHYGVFTCEGCKSFFKRSIRRNLSYTCRSNRDCQIDQHHRNQCQYCRLKKCFRVGMRKEAVQRGRIPHSLPGAVAASSGSPPGSALAAVASGGDLFPGQPVSELIAQLLRAEPYPAAAGRFGAGGGAAGAVLGIDNVCELAWDPRNEMGASGDMRAANLWPSPLVIKHTKKNSPALSLTADQMVSALLDAEPPMIYSEYDPSRPFSEASMMGLLTNLADRELVHMINWAKRVPGFGDLNLHDQVHLLECAWLEILMIGLVWRSMEHPGKLLFAPNLLLDRNQGKCVEGMVEIFDMLLATSSRFRMMNLQGEEFVCLKSIILLNSGVYTFLSSTLKSLEEKDHIHRVLDKITDTLIHLMAKAGLTLQQQHRRLAQLLLILSHIRHMSNKGMEHLYNMKCKNVVPLYDLLLEMLDAHRLHAPASRMGVPPEEPSQTQLATTSSTSAHSLQTYYIPPEAEGFPNTI

SEQ ID NO: 11 - DNA, 1590 bp, artificial sequence
  /note=“Description of artificial sequence coding sequence of S83A mutated NR2F6 ER-LBD fusion mutant”
atggccatggtgaccggcggctggggcggccccggcggcgacacgaacggcgtggacaaggcgggcggctacccgcgcgcggccgaggacgactcggcctcgccccccggtgccgccagcgacgccgagccgggcgacgaggagcggccggggctgcaggtggactgcgtggtgtgcggggacaagtcgagcggcaagcattacggtgtcttcacctgcgagggctgcaagagctttttcaagcgagccatccgccgcaacctcagctacacctgccggtccaaccgtgactgccagatcgaccagcaccaccggaaccagtgccagtactgccgtctcaagaagtgcttccgggtgggcatgaggaaggaggcggtgcagcgcggccgcatcccgcactcgctgcctggtgccgtggccgcctcctcgggcagccccccgggctcggcgctggcggcagtggcgagcggcggagacctcttcccggggcagccggtgtccgaactgatcgcgcagctgctgcgcgctgagccctaccctgcggcggccggacgcttcggcgcagggggcggcgcggcgggcgcggtgctgggcatcgacaacgtgtgcgagctggcggcgtgggatccacgaaatgaaatgggtgcttcaggagacatgagggctgccaacctttggccaagccctcttgtgattaagcacactaagaagaatagccctgccttgtccttgacagctgaccagatggtcagtgccttgttggatgctgaaccgcccatgatctattctgaatatgatccttctagacccttcagtgaagcctcaatgatgggcttattgaccaacctagcagatagggagctggttcatatgatcaactgggcaaagagagtgccaggctttggggacttgaatctccatgatcaggtccaccttctcgagtgtgcctggctggagattctgatgattggtctcgtctggcgctccatggaacacccggggaagctcctgtttgctcctaacttgctcctggacaggaatcaaggtaaatgtgtggaaggcatggtggagatctttgacatgttgctggctacgtcaagtcggttccgcatgatgaacctgcagggagaagagtttgtgtgcctcaaatccatcattttgcttaattccggagtgtacacgtttctgtccagcaccttgaagtctctggaagagaaggaccacatccaccgtgtcctggacaagatcacagacactttgatccacctgatggccaaagctggcctgactctgcagcagcagcatcgccgcctagctcagctccttctcattctttcccatatccggcacatgagtaacaaaggcatggagcatctctacaacatgaaatgcaagaacgttgtgcccctctatgacctgctcctggagatgttggatgcccaccgccttcatgccccagccagtcgcatgggagtgcccccagaggagcccagccagacccagctggccaccaccagctccacttcagcacattccttacaaacctactacatacccccggaagcagagggcttccccaacacgatctga

SEQ ID NO: 12 - protein, 528 aa, artificial sequence
  /note=“Description of artificial sequence amino acid sequence of coding sequence of S83A mutated NR2F6 ER-LBD fusion mutant”
MAMVTGGWGGPGGDTNGVDKAGGYPRAAEDDSASPPGAASDAEPGDEERPGLQVDCVVCGDKSSGKHYGVFTCEGCKSFFKRAIRRNLSYTCRSNRDCQIDQHHRNQCQYCRLKKCFRVGMRKEAVQRGRIPHSLPGAVAASSGSPPGSALAAVASGGDLFPGQPVSELIAQLLRAEPYPAAAGRFGAGGGAAGAVLGIDNVCELAWDPRNEMGASGDMRAANLWPSPLVIKHTKKNSPALSLTADQMVSALLDAEPPMIYSEYDPSRPFSEASMMGLLTNLADRELVHMINWAKRVPGFGDLNLHDQVHLLECAWLEILMIGLVWRSMEHPGKLLFAPNLLLDRNQGKCVEGMVEIFDMLLATSSRFRMMNLQGEEFVCLKSIILLNSGVYTFLSSTLKSLEEKDHIHRVLDKITDTLIHLMAKAGLTLQQQHRRLAQLLLILSHIRHMSNKGMEHLYNMKCKNVVPLYDLLLEMLDAHRLHAPASRMGVPPEEPSQTQLATTSSTSAHSLQTYYIPPEAEGFPNTI

SEQ ID NO: 13 - DNA, 1590 bp, artificial sequence
  /note=“Description of artificial sequence coding sequence of S83E mutated NR2F6 ER-LBD fusion mutant”
atggccatggtgaccggcggctggggcggccccggcggcgacacgaacggcgtggacaaggcgggcggctacccgcgcgcggccgaggacgactcggcctcgccccccggtgccgccagcgacgccgagccgggcgacgaggagcggccggggctgcaggtggactgcgtggtgtgcggggacaagtcgagcggcaagcattacggtgtcttcacctgcgagggctgcaagagctttttcaagcgagagatccgccgcaacctcagctacacctgccggtccaaccgtgactgccagatcgaccagcaccaccggaaccagtgccagtactgccgtctcaagaagtgcttccgggtgggcatgaggaaggaggcggtgcagcgcggccgcatcccgcactcgctgcctggtgccgtggccgcctcctcgggcagccccccgggctcggcgctggcggcagtggcgagcggcggagacctcttcccggggcagccggtgtccgaactgatcgcgcagctgctgcgcgctgagccctaccctgcggcggccggacgcttcggcgcagggggcggcgcggcgggcgcggtgctgggcatcgacaacgtgtgcgagctggcggcgtgggatccacgaaatgaaatgggtgcttcaggagacatgagggctgccaacctttggccaagccctcttgtgattaagcacactaagaagaatagccctgccttgtccttgacagctgaccagatggtcagtgccttgttggatgctgaaccgcccatgatctattctgaatatgatccttctagacccttcagtgaagcctcaatgatgggcttattgaccaacctagcagatagggagctggttcatatgatcaactgggcaaagagagtgccaggctttggggacttgaatctccatgatcaggtccaccttctcgagtgtgcctggctggagattctgatgattggtctcgtctggcgctccatggaacacccggggaagctcctgtttgctcctaacttgctcctggacaggaatcaaggtaaatgtgtggaaggcatggtggagatctttgacatgttgctggctacgtcaagtcggttccgcatgatgaacctgcagggagaagagtttgtgtgcctcaaatccatcattttgcttaattccggagtgtacacgtttctgtccagcaccttgaagtctctggaagagaaggaccacatccaccgtgtcctggacaagatcacagacactttgatccacctgatggccaaagctggcctgactctgcagcagcagcatcgccgcctagctcagctccttctcattctttcccatatccggcacatgagtaacaaaggcatggagcatctctacaacatgaaatgcaagaacgttgtgcccctctatgacctgctcctggagatgttggatgcccaccgccttcatgccccagccagtcgcatgggagtgcccccagaggagcccagccagacccagctggccaccaccagctccacttcagcacattccttacaaacctactacatacccccggaagcagagggcttccccaacacgatctga

SEQ ID NO: 14 - protein, 528 aa, artificial sequence
  /note=“Description of artificial sequence binding buffer double-stranded oligonucelotide probes”
MAMVTGGWGGPGGDTNGVDKAGGYPRAAEDDSASPPGAASDAEPGDEERPGLQVDCVVCGDKSSGKHYGVFTCEGCKSFFKREIRRNLSYTCRSNRDCQIDQHHRNQCQYCRLKKCFRVGMRKEAVQRGRIPHSLPGAVAASSGSPPGSALAAVASGGDLFPGQPVSELIAQLLRAEPYPAAAGRFGAGGGAAGAVLGIDNVCELAWDPRNEMGASGDMRAANLWPSPLVIKHTKKNSPALSLTADQMVSALLDAEPPMIYSEYDPSRPFSEASMMGLLTNLADRELVHMINWAKRVPGFGDLNLHDQVHLLECAWLEILMIGLVWRSMEHPGKLLFAPNLLLDRNQGKCVEGMVEIFDMLLATSSRFRMMNLQGEEFVCLKSIILLNSGVYTFLSSTLKSLEEKDHIHRVLDKITDTLIHLMAKAGLTLQQQHRRLAQLLLILSHIRHMSNKGMEHLYNMKCKNVVPLYDLLLEMLDAHRLHAPASRMGVPPEEPSQTQLATTSSTSAHSLQTYYIPPEAEGFPNTI

SEQ ID NO: 15 - DNA, 24 bp, artificial sequence
  /note=“Description of artificial sequence binding buffer double-stranded oligonucelotide probes”
gccatggggggatccccgaagtcc

SEQ ID NO: 16 - DNA, 21 bp, artificial sequence
  /note=“Description of artificial sequence binding buffer double-stranded oligonucelotide probes”
cgcttgatgactcagccggaa

SEQ ID NO: 17 - DNA, 29 bp, artificial sequence
  /note=“Description of artificial sequence binding buffer double-stranded oligonucelotide probes”
gcccaaagaggaaaatttgtttcatacag

SEQ ID NO: 18 - DNA, 24 bp, artificial sequence
  /note=“Description of artificial sequence binding buffer double-stranded oligonucelotide probes”
gtgtcaaaggtcgtgtcaaaggtc

SEQ ID NO: 19 - RNA, 21 nt, artificial sequence
  /note=“Description of artificial sequence siGenome ON-TARGETplus SMARTpool duplex (9), J-045088-09,NR2F6, sense sequence”
guggaaagcauuacggcguuu

SEQ ID NO: 20 - RNA, 21 nt, artificial sequence
  /note=“Description of artificial sequence siGenome ON-TARGETplus SMARTpool duplex (9), J-045088-09,NR2F6, antisense sequence”
acgccguaaugcuuuccacuu

SEQ ID NO: 21 - RNA, 21 nt, artificial sequence
  /note=“Description of artificial sequence siGenome ON-TARGETplus SMARTpool duplex (10), J-045088-10, NR2F6, sense sequence”
agguggaugcugcggaguauu

SEQ ID NO: 22 - RNA, 21 nt, artificial sequence
  /note=“Description of artificial sequence siGenome ON-TARGETplus SMARTpool duplex (10), J-045088-10,NR2F6, antisense sequence”
uacuccgcagcauccaccuuu

SEQ ID NO: 23 - RNA, 21 nt, artificial sequence
  /note=“Description of artificial sequence siGenome ON-TARGETplus SMARTpool duplex (11), J-045088-11, NR2F6, sense sequence”
gcaucgacaacgugugcgauu

SEQ ID NO: 24 - RNA, 21 nt, artificial sequence
  /note=“Description of artificial sequence siGenome ON-TARGETplus SMARTpool duplex (11), J-045088-11,NR2F6, antisense sequence”
ucgcacacguugucgaugcuu

SEQ ID NO: 25 - RNA, 21 nt, artificial sequence
  /note=“Description of artificial sequence siGenome ON-TARGETplus SMARTpool duplex (12), J-045088-12,NR2F6, sense sequence”
ggcaagacacccaucgagauu

SEQ ID NO: 26 - RNA, 21 nt, artificial sequence
  /note=“Description of artificial sequence siGenome ON-TARGETplus SMARTpool duplex (12), J-045088-12, NR2F6, antisense sequence”
ucucgaugggugucuugccuu

SEQ ID NO: 27 - DNA, 4044 bp, artificial sequence
  /note=“Description of artificial sequence pRL-TK RENILLA Vector sequence”
agatctaaatgagtcttcggacctcgcgggggccgcttaagcggtggttagggtttgtctgacgcggggggagggggaaggaacgaaacactctcattcggaggcggctcggggtttggtcttggtggccacgggcacgcagaagagcgccgcgatcctcttaagcacccccccgccctccgtggaggcgggggtttggtcggcgggtggtaactggcgggccgctgactcgggcgggtcgcgcgccccagagtgtgaccttttcggtctgctcgcagacccccgggcggcgccgccgcggcggcgacgggctcgctgggtcctaggctccatggggaccgtatacgtggacaggctctggagcatccgcacgactgcggtgatattaccggagaccttctgcgggacgagccgggtcacgcggctgacgcggagcgtccgttgggcgacaaacaccaggacggggcacaggtacactatcttgtcacccggaggcgcgagggactgcaggagcttcagggagtggcgcagctgcttcatccccgtggcccgttgctcgcgtttgctggcggtgtccccggaagaaatatatttgcatgtctttagttctatgatgacacaaaccccgcccagcgtcttgtcattggcgaattcgaacacgcagatgcagtcggggcggcgcggtcccaggtccacttcgcatattaaggtgacgcgtgtggcctcgaacaccgagcgaccctgcagcgacccgcttaaaagcttgattcttctgacacaacagtctcgaacttaagctgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagctcttaaggctagagtacttaatacgactcactataggctagccaccatgacttcgaaagtttatgatccagaacaaaggaaacggatgataactggtccgcagtggtgggccaatgtaaacaaatgaatgttcttgattcatttattaattattatgattcagaaaaacatgcagaaaatgctgttatttttttacatggtaacgcggcctcttcttatttatggcgacatgttgtgccacatattgagccagtagcgcggtgtattataccagaccttattggtatgggcaaatcaggcaaatctggtaatggttcttataggttacttgatcattacaaatatcttactgcatggtttgaacttcttaatttaccaaagaagatcatttttgtcggccatgattggggtgcttgtttggcatttcattatagctatgagcatcaagataagatcaaagcaatagttcacgctgaaagtgtagtagatgtgattgaatcatgggatgaatggcctgatattgaagaagatattgcgttgatcaaatctgaagaaggagaaaaaatggttttggagaataacttcttcgtggaaaccatgttgccatcaaaaatcatgagaaagttagaaccagaagaatttgcagcatatcttgaaccattcaaagagaaaggtgaagttcgtcgtccaacattatcatggcctcgtgaaatcccgttagtaaaaggtggtaaacctgacgttgtacaaattgttaggaattataatgcttatctacgtgcaagtgatgatttaccaaaaatgtttattgaatcggacccaggattcttttccaatgctattgttgaaggtgccaagaagtttcctaatactgaatttgtcaaagtaaaaggtcttcatttttcgcaagaagatgcacctgatgaaatgggaaaatatatcaaatcgttcgttgagcgagttctcaaaaatgaacaataattctagagcggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtaaaatcgataaggatccaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatggctcgac

SEQ ID NO: 28 - DNA, 29 bp, artificial sequence
  /note=“Description of artificial sequenceNF-AT/AP-1 Consensus Oligonucleotide from IL-2 distal site (3 repeats)”
gcccaaagaggaaaatttgtttcatacag

SEQ ID NO: 29 - DNA, 1159 bp, Homo sapiens
  Description “Homo sapiens interleukin17 (IL17) gene, exon 1 promoter region and partial cds”
ggatctcaggacaaacaggttcaggatccggcaaactaattttacactccagccattgagttggaactactggccagcctccccccgagttagcatgtagaatatgggataccagctgagtgcctgagagttatcattcacctcagtgggggtaggggcggagaagggtgacatatagccagccacatctatatccactggcccttccttgtcctagtcctctgtattcctgagaaggaactattctcaaggacctgagtccaagttcatcttacttagagtacagagaaaagaaccgctaactccttctctctttcccccatcatgtctcctctcctttctagttctcatcactctctactcccccctgccccccttttctccatctccatcacctttgtccagtctctatccccattttcaattccttcctcaaaacaccaagttgcttggtagcatgcagggttggaacatgcctttaacagaaaatctcgtgtctcttgaacctagttatttattccttgagcagagtagatattcaacaaaagaattgttaaattcaattaaataggatatatcttattattaaatatttttttcattttttgtttacttatatgatgggaacttgagtagtttccggaattgtctccacaacacctggccaaggaatctgtgaggaaaagaaagatcaaatggaaaatcaaggtacatgacaccagaagacctacatgttacttcaaactttttcttcctcatgaaccattaaaatagagcataactcttctggcagctgtacatatgttcataaatacatgatattgacccatagcatagcagctctgctcagcttctaacaagtaagaatgaaaagaggacatggtctttaggaacatgaatttctgcccttcccattttccttcagaaggagagattcttctatgacctcattgggggcggaaattttaaccaaaatggtgtcacccctgaacccactgcgacacgccacgtaagtgaccacagaaggagaaaagccctataaaaagagagacgatagcgctacattttgtccatctcatagcaggcacaaactcatccatccccagttgattggaagaaacaacgatgactcctgggaagacctcattggtggtgagtc

SEQ ID NO: 30 - DNA, 1804 bp, artificial sequence
  /note=“Description of artificial sequence coding sequence of C112S mutated NR2F6”
gtgcagcccgtgccccccgcgcgccggggccgaatgcgcgccgcgtagggtcccccgggccgagaggggtgcccggagggaagagcgcggtgggggcgccccggccccgctgccctggggctatggccatggtgaccggcggctggggcggccccggcggcgacacgaacggcgtggacaaggcgggcggctacccgcgcgcggccgaggacgactcggcctcgccccccggtgccgccagcgacgccgagccgggcgacgaggagcggccggggctgcaggtggactgcgtggtgtgcggggacaagtcgagcggcaagcattacggtgtcttcacctgcgagggctgcaagagctttttcaagcgaagcatccgccgcaacctcagctacacctgccggtccaaccgtgactgccagatcgaccagcaccaccggaaccagtgccagtctggccgtctcaagaagtgcttccgggtgggcatgaggaaggaggcggtgcagcgcggccgcatcccgcactcgctgcctggtgccgtggccgcctcctcgggcagccccccgggctcggcgctggcggcagtggcgagcggcggagacctcttcccggggcagccggtgtccgaactgatcgcgcagctgctgcgcgctgagccctaccctgcggcggccggacgcttcggcgcagggggcggcgcggcgggcgcggtgctgggcatcgacaacgtgtgcgagctggcggcgcggctgctcttcagcaccgtggagtgggcgcgccacgcgcccttcttccccgagctgccggtggccgaccaggtggcgctgctgcgcctgagctggagcgagctcttcgtgctgaacgcggcgcaggcggcgctgcccctgcacacggcgccgctactggccgccgccggcctccacgccgcgcctatggccgccgagcgcgccgtggctttcatggaccaggtgcgcgccttccaggagcaggtggacaagctgggccgcctgcaggtcgactcggccgagtatggctgcctcaaggccatcgcgctcttcacgcccgacgcctgtggcctctcagacccggcccacgttgagagcctgcaggagaaggcgcaggtggccctcaccgagtatgtgcgggcgcagtacccgtcccagccccagcgcttcgggcgcctgctgctgcggctccccgccctgcgcgcggtccctgcctccctcatctcccagctgttcttcatgcgcctggtggggaagacgcccattgagacactgatcagagacatgctgctgtcggggagtaccttcaactggccctacggctcgggccagtgaccatgacggggccacgtgtgctgtggccaggcctgcagacagacctcaagggacagggaatgctgaggcctcgaggggcctcccggggcccaggactctggcttctctcctcagacttctattttttaaagactgtgaaatgtttgtcttttctgttttttaaatgatcatgaaaccaaaaagagactgatcatccaggcctcagcctcatcctccccaggacccctgtccaggatggagggtccaatcctaggacagccttgttcctcagcacccctagcatgaacttgtgggatggtggggttggcttccctggcatgatggacaaaggcctggcgtcggccagaggggctgctccagtgggcaggggtagctagcgtgtgccaggcagatcctctggacacgtaacctatgtcagacactacatgatgactcaaggccaataataaagacatttcctacctgca

SEQ ID NO: 31 - protein, 404 aa, artificial sequence
  /note=“Description of artificial sequence amino acid sequence of C112S mutated NR2F6”
MAMVTGGWGGPGGDTNGVDKAGGYPRAAEDDSASPPGAASDAEPGDEERPGLQVDCVVCGDKSSGKHYGVFTCEGCKSFFKRSIRRNLSYTCRSNRDCQIDQHHRNQCQYSRLKKCFRVGMRKEAVQRGRIPHSLPGAVAASSGSPPGSALAAVASGGDLFPGQPVSELIAQLLRAEPYPAAAGRFGAGGGAAGAVLGIDNVCELAARLLFSTVEWARHAPFFPELPVADQVALLRLSWSELFVLNAAQAALPLHTAPLLAAAGLHAAPMAAERAVAFMDQVRAFQEQVDKLGRLQVDSAEYGCLKAIALFTPDACGLSDPAHVESLQEKAQVALTEYVRAQYPSQPQRFGRLLLRLPALRAVPASLISQLFFMRLVGKTPIETLIRDMLLSGSTFNWPYGSGQ

SEQ ID NO: 32 - DNA, 1653 bp, Photinus pyralis
  Description “nucleic acid sequence of luciferase of Photinus pyralis (common eastern firefly luciferase)”
atggaagacgccaaaaacataaagaaaggcccggcgccattctatcctctagaggatggaaccgctggagagcaactgcataaggctatgaagagatacgccctggttcctggaacaattgcttttacagatgcacatatcgaggtgaacatcacgtacgcggaatacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgaacatttcgcagcctaccgtagtgtttgtttccaaaaaggggttgcaaaaaattttgaacgtgcaaaaaaaattaccaataatccagaaaattattatcatggattctaaaacggattaccagggatttcagtcgatgtacacgttcgtcacatctcatctacctcccggttttaatgaatacgattttgtaccagagtcctttgatcgtgacaaaacaattgcactgataatgaattcctctggatctactgggttacctaagggtgtggcccttccgcatagaactgcctgcgtcagattctcgcatgccagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttttacgatcccttcaggattacaaaattcaaagtgcgttgctagtaccaaccctattttcattcttcgccaaaagcactctgattgacaaatacgatttatctaatttacacgaaattgcttctgggggcgcacctctttcgaaagaagtcggggaagcggttgcaaaacgcttccatcttccagggatacgacaaggatatgggctcactgagactacatcagctattctgattacacccgagggggatgataaaccgggcgcggtcggtaaagttgttccattttttgaagcgaaggttgtggatctggataccgggaaaacgctgggcgttaatcagagaggcgaattatgtgtcagaggacctatgattatgtccggttatgtaaacaatccggaagcgaccaacgccttgattgacaaggatggatggctacattctggagacatagcttactgggacgaagacgaacacttcttcatagttgaccgcttgaagtctttaattaaatacaaaggatatcaggtggcccccgctgaattggaatcgatattgttacaacaccccaacatcttcgacgcgggcgtggcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagtccaaattgtaa

SEQ ID NO: 33 - protein, 550 aa, Photinus pyralis
  Description “amino acid sequence of luciferase of Photinus pyralis (common eastern firefly luciferase)”
MEDAKNIKKGPAPFYPLEDGTAGEQLHKAMKRYALVPGTIAFTDAHIEVNITYAEYFEMSVRLAEAMKRYGLNTNHRIVVCSENSLQFFMPVLGALFIGVAVAPANDIYNERELLNSMNISQPTVVFVSKKGLQKILNVQKKLPIIQKIIIMDSKTDYQGFQSMYTFVTSHLPPGFNEYDFVPESFDRDKTIALIMNSSGSTGLPKGVALPHRTACVRFSHARDPIFGNQIIPDTAILSVVPFHHGFGMFTTLGYLICGFRVVLMYRFEEELFLRSLQDYKIQSALLVPTLFSFFAKSTLIDKYDLSNLHEIASGGAPLSKEVGEAVAKRFHLPGIRQGYGLTETTSAILITPEGDDKPGAVGKVVPFFEAKVVDLDTGKTLGVNQRGELCVRGPMIMSGYVNNPEATNALIDKDGWLHSGDIAYWDEDEHFFIVDRLKSLIKYKGYQVAPAELESILLQHPNIFDAGVAGLPDDDAGELPAAVVVLEHGKTMTEKEIVDYVASQVTTAKKLRGGVVFVDEVPKGLTGKLDARKIREILIKAKKGGKSKL

(19) United States
(12) Patent Application Publication
(10) Pub. No.: US 2011/0236427 A1
(43) Pub. Date: Sep. 29, 2011

(54) Antagonists of NR2F6 For Augmenting The Immune Response

(75) Inventors: Gottfried Baier, Igls/Innsbruck (AT); Natascha Hermann-Kleiter, Innsbruck (AT)

(73) Assignee: Medizinische Universitat Innsbruck, Innsbruck (AT)

(21) Appl. No.: 13/004,781

(22) Filed: Jan. 11, 2011

Related U.S. Application Data
(63) Continuation of application No. PCT/EP2009/058881, filed on Jul. 10, 2009.

(30) Foreign Application Priority Data
Jul. 11, 2008 (EP) 08012631.1

(51) Int. Cl. A61K 39/00; C07H 21/02; C07H 21/04; C07K 2/00; A61P 35/00; A61P 37/04; C12Q 1/02; C12Q 1/68; A01K 67/033; C12N 15/63; C07K 16/00; G01N 33/00

(52) U.S. Cl. 424/277.1; 536/24.5; 536/23.1; 530/300; 435/29; 435/6.13; 800/10; 435/320.1; 530/387.1; 800/3

(57) ABSTRACT

The present invention relates to antagonists/inhibitors of NR2F6 (nuclear orphan receptor receptor Ear2) for the treatment of a disease related to an insufficient immune response. Furthermore, pharmaceutical compositions comprising said antagonists/inhibitors of NR2F6 and a pharmaceutical carrier are comprised. In a further aspect, the present invention provides for a method for identifying immunoaugmenting agents comprising contacting a cell, tissue or a non-human animal comprising a reporter construct for NR2F6-inhibition with a candidate molecule, measuring the reporter signal and selecting a candidate molecule which alters the reporter signal. Furthermore, the present invention relates to the non-human transgenic animals or cells or tissue derived therefrom useful in the provided methods for identifying immunoaugmenting agents. In yet another aspect, the present invention relates to ligand-mediated reporter gene expression constructs, ligand displacement constructs, fluorescent cellular sensor fusion mutant constructs and ligand-induced homo- and/or heterodimer constructs useful in the provided methods for identifying immunoaugmenting agents.

The present invention relates to antagonists/inhibitors of NR2F6 (nuclear orphan receptor receptor Ear2) for the treatment of a disease related to an insufficient immune response. Furthermore, pharmaceutical compositions comprising said antagonists/inhibitors of NR2F6 and a pharmaceutical carrier are comprised. In a further aspect, the present invention provides for a method for identifying immunoaugmenting agents comprising contacting a cell, tissue or a non-human animal comprising a reporter construct for NR2F6-inhibition with a candidate molecule, measuring the reporter signal and selecting a candidate molecule which alters the reporter signal. Furthermore, the present invention relates to the non-human transgenic animals or cells or tissue derived therefrom useful in the provided methods for identifying immunoaugmenting agents. In yet another aspect, the present invention relates to ligand-mediated reporter gene expression constructs, ligand displacement constructs, fluorescent cellular sensor fusion mutant constructs and ligand-induced homo- and/or heterodimer constructs useful in the provided methods for identifying immunoaugmenting agents.

Immune responses are exquisitely controlled, requiring multiple finely tuned levels of activation as well as inactivation signals. In T lymphocytes among these signalling networks, T cell receptor (TCR) stimulation activates NF-AT, a family of transcription factors that is of particular importance during immune cell activation. NF-AT mediates the transcriptional induction of “cell fate-determining genes”, which govern as diverse outcomes as activation, anergy or apoptosis (Crabtree (2002) Cell 109 Suppl:S67-79; Hogan (2003) Genes & development 17:2205-2232; Heissmeyer (2004) Nature immunology 5:255-265). Mechanistically, the rise of intracellular Ca²⁺ triggered by antigen binding to the TCR leads to the activation of calcineurin's phosphatase activity. This leads to dephosphorylation of phopsho-sites within the N-terminal regulatory domain on NF-AT and, subsequently, nuclear import of NF-AT. Upon transient stimuli, however, feedback inhibition, mediated via GSK3 (glycogen synthase kinase 3), CK1 (casein kinase 1) and DYRK (dual-specificity tyrosine phosphorylation-regulated kinase) protein kinases are known to counter-regulate NF-AT nuclear occupancy by rephosphorylation (Gwack (2006) Nature 441:646-650), which induces the nuclear export of NF-AT and the abort of immune activation-associated gene transcription.

NF-AT family members are also subject to regulation in the nucleus through their ability to directly interact with other transcriptional regulatory factors. NF-AT is known to require a protein partner for high-affinity binding at most DNA sites. NF-AT complexes mostly contain cell type- or cell lineage-specific protein binding partners. In cardiac, skeletal, and smooth muscle cells, NF-AT forms complexes with GATA proteins (Hogan (2003) loc. cit). In the regulatory T cell lineage (Treg), an interaction of NF-AT and forkhead transcription factor FOXP3 occurs (Wu (2006) Cell 126:375-387), whereas in effector T cells, NF-AT forms complexes with activator protein 1 (Fos-Jun) proteins (AP-1; Rao (1997) Annual review of immunology 15:707-747). In effector T cells the Ca²⁺/calcineurin/NF-AT pathway interacts with the Ras/MAPK/AP-1 signalling pathway to regulate downstream target gene expression such as interleukin 2 (Macian (2001) Oncogene 20:2476-2489), which contains NF-AT/AP-1 DNA binding elements within the respective promoter sequences. NF-AT also interacts with known suppressor proteins such as “inducible cAMP early repressor” (ICER; Bodor (1996) PNAS 93:3536-3541) and “21-kDa small nuclear factor isolated from T cells” (p21SNFT; Bower (2002) J Biol Chem 277:34967-34977).

The detailed control of NF-AT within the nucleus, however, is not yet understood and positive and/or negative functional interactions of NF-AT with other yet-to-be-identified transcription factors may very well occur during T cell activation processes.

The technical problem of the present invention is the provision of means and methods for the medical or pharmaceutical intervention of immunological diseases in particular of diseases/disorders related to an insufficient immune response. The solution to the above technical problem is achieved by providing the embodiments characterized in the claims.

Accordingly, the present invention relates to antagonists/inhibitors of NR2F6 for the treatment of a disease related to an insufficient immune response. Moreover, the present invention relates to the use of an antagonist/inhibitor of NR2F6 for the preparation of a medicament for the treatment of a disease related to an insufficient immune response.

As demonstrated in the provided Examples, it was surprisingly found that antigen receptor stimulation-induced NF-AT/AP-1 activity is regulated through the nuclear receptor NR2F6. NR2F6 deficient mice (NR2F6^{-/-} mice) have previously been reported to show defects in development of the brain, namely the locus coeruleus, associated with defects in circadian behaviour and circadian gene expression. Furthermore, NR2F6^{-/-} mice are also more pain sensitive due to a lower noradrenalin concentration in the spinal cord (Warnecke (2005) Genes & development 19:614-625). In contrast to the above, however, it was surprisingly found in the context of the present invention that NR2F6 deficient mice also show a disturbed immune response. More particularly, it was surprisingly found that by preventing NR2F6 function, transcriptional activation of NF-AT/AP-1 is enhanced in immune cells which leads to an augmented immune response.

In the context of the present invention, the term “NR2F6” relates to “nuclear receptor subfamily 2, group F, member 6” or “Ear2” as described in the art. Nuclear receptors are transcription factors that regulate the expression of specific target genes, thereby orchestrating a wide array of cellular processes including cellular activation, development and disease progression (Moore (2006) Chem Med Chem 1:504-523). The nuclear receptor super-family consists of receptors that bind to hormones and orphan receptors with yet undefined endogenous ligands. The COUP-TF orphan receptors are known to be preferentially expressed in the central nervous system and organs that depend on the interaction between mesenchyme and epithelial layers (Giguere (1999) Endocr Rev 20:689-725; Miyajima (1988) Nucleic Acids Res 16:11057-11074; Qiu (1997) Genes & development 11:1925-1937; Zhang (2004) Vitam Horm 68:1-48; Armentano (2006) Development 133:4151-4162; You (2005) Nature 435:98-104; Takamoto (2005) Development 132:2179-2189; Tripodi (2004) Development 131:6119-6129).

The three mammalian COUP-TF family members are NR2F1/Ear3, NR2F2/Arp1 and NR2F6 (Miyajima (1988) loc. cit.; Qiu (1995) Genomics 29:240-246; Ladias (1991) Science 251:561-565). The established target genes of said COUP-TF family members are apolipoproteins and retinoic acid-, peroxisome-, oxytocin-, estrogen- and vitamin D receptors (reviewed in Giguere (1999) loc. cit.; Moore (2006) loc. cit.). By yeast 1-hybrid screen and in vitro assays with recombinant NR2F6 it was found that the TGACCT direct-repeat motif is the DNA binding sequence of NR2F6 and that overexpression of NR2F6 induces repression of the renin gene transcription in a DNA-binding-specific manner (Liu (2003) Circ Res 92:1033-1040).

The coding regions of NR2F6 or functional fragments thereof are known in the art and comprise, inter alia, the NR2F6 GenBank entries “X12794”, “NM_{—}005234”, “BC084544”, “BC063018” and “BC002669” for Homo sapiens NR2F6 or Homo sapiens NR2F6 fragments; “NM_{—}139113” for Rattus norvegicus NR2F6 or Rattus norvegicus NR2F6 fragments; “NM_{—}010150” for Mus musculus NR2F6 or Mus musculus NR2F6 fragments; “BV448755” and “BV448156” for Macaca mulatto NR2F6 or Macaca mulatto NR2F6 fragments; “BC074651” for Xenopus tropicalis NR2F6 or Xenopus tropicalis NR2F6 fragments; “NM_{—}001086712” and “BC056043” for Xenopus laevis NR2F6 or Xenopus laevis NR2F6 fragments and “AY702439” for Gallus gallus NR2F6 or Gallus gallus NR2F6 fragments. The person skilled in the art may easily deduce the relevant coding region of NR2F6 in these GenBank entries, which may also comprise the entry of genomic DNA as well as mRNA/cDNA (see also FIG. 1).

In particular, wild type human NR2F6 may be encoded by the following nucleic acid sequence (start codon in bold):

| (SEQ ID NO: 1) | |
|---|---|
| 1 | gtgcagcccg tgccccccgc gcgccggggc cgaatgcgcg ccgcgtaggg tcccccgggc |
| 61 | cgagaggggt gcccggaggg aagagcgcgg tgggggcgcc ccggccccgc tgccctgggg |
| 121 | ctatggccat ggtgaccggc ggctggggcg gccccggcgg cgacacgaac ggcgtggaca |
| 181 | aggcgggcgg ctacccgcgc gcggccgagg acgactcggc ctcgcccccc ggtgccgcca |
| 241 | gcgacgccga gccgggcgac gaggagcggc cggggctgca ggtggactgc gtggtgtgcg |
| 301 | gggacaagtc gagcggcaag cattacggtg tcttcacctg cgagggctgc aagagctttt |
| 361 | tcaagcgaag catccgccgc aacctcagct acacctgccg gtccaaccgt gactgccaga |
| 421 | tcgaccagca ccaccggaac cagtgccagt actgccgtct caagaagtgc ttccgggtgg |
| 481 | gcatgaggaa ggaggcggtg cagcgcggcc gcatcccgca ctcgctgcct ggtgccgtgg |
| 541 | ccgcctcctc gggcagcccc ccgggctcgg cgctggcggc agtggcgagc ggcggagacc |
| 601 | tcttcccggg gcagccggtg tccgaactga tcgcgcagct gctgcgcgct gagccctacc |
| 661 | ctgcggcggc cggacgcttc ggcgcagggg gcggcgcggc gggcgcggtg ctgggcatcg |
| 721 | acaacgtgtg cgagctggcg gcgcggctgc tcttcagcac cgtggagtgg gcgcgccacg |
| 781 | cgcccttctt ccccgagctg ccggtggccg accaggtggc gctgctgcgc ctgagctgga |
| 841 | gcgagctctt cgtgctgaac gcggcgcagg cggcgctgcc cctgcacacg gcgccgctac |
| 901 | tggccgccgc cggcctccac gccgcgccta tggccgccga gcgcgccgtg gctttcatgg |
| 961 | accaggtgcg cgccttccag gagcaggtgg acaagctggg ccgcctgcag gtcgactcgg |
| 1021 | ccgagtatgg ctgcctcaag gccatcgcgc tcttcacgcc cgacgcctgt ggcctctcag |
| 1081 | acccggccca cgttgagagc ctgcaggaga aggcgcaggt ggccctcacc gagtatgtgc |
| 1141 | gggcgcagta cccgtcccag ccccagcgct tcgggcgcct gctgctgcgg ctccccgccc |
| 1201 | tgcgcgcggt ccctgcctcc ctcatctccc agctgttctt catgcgcctg gtggggaaga |
| 1261 | cgcccattga gacactgatc agagacatgc tgctgtcggg gagtaccttc aactggccct |
| 1321 | acggctcggg ccagtgacca tgacggggcc acgtgtgctg tggccaggcc tgcagacaga |
| 1381 | cctcaaggga cagggaatgc tgaggcctcg aggggcctcc cggggcccag gactctggct |
| 1441 | tctctcctca gacttctatt ttttaaagac tgtgaaatgt ttgtcttttc tgttttttaa |
| 1501 | atgatcatga aaccaaaaag agactgatca tccaggcctc agcctcatcc tccccaggac |
| 1561 | ccctgtccag gatggagggt ccaatcctag gacagccttg ttcctcagca cccctagcat |
| 1621 | gaacttgtgg gatggtgggg ttggcttccc tggcatgatg gacaaaggcc tggcgtcggc |
| 1681 | cagaggggct gctccagtgg gcaggggtag ctagcgtgtg ccaggcagat cctctggaca |
| 1741 | cgtaacctat gtcagacact acatgatgac tcaaggccaa taataaagac atttcctacc |
| 1801 | tgca |

which corresponds to the following amino acid sequence:

(SEQ ID NO: 2)
MAMVTGGWGGPGGDTNGVDKAGGYPRAAEDDSASPPGAASDAEPGDEE
RPGLQVDCVVCGDKSSGKHYGVFTCEGCKSFFKRSIRRNLSYTCRSNR
DCQIDQHHRNQCQYCRLKKCFRVGMRKEAVQRGRIPHSLPGAVAASSG
SPPGSALAAVASGGDLFPGQPVSELIAQLLRAEPYPAAAGRFGAGGGA
AGAVLGIDNVCELAARLLFSTVEWARHAPFFPELPVADQVALLRLSWS
ELFVLNAAQAALPLHTAPLLAAAGLHAAPMAAERAVAFMDQVRAFQEQ
VDKLGRLQVDSAEYGCLKAIALFTPDACGLSDPAHVESLQEKAQVALT
EYVRAQYPSQPQRFGRLLLRLPALRAVPASLISQLFFMRLVGKTPIET
LIRDMLLSGSTFNWPYGSGQ

Accordingly, the NR2F6 molecules to be employed in the context of the present invention comprise, but are not limited to the molecules encoded by the nucleic acid molecules as described herein. Also envisaged are NR2F6 orthologs which are at least 60%, 70%, 80%, 90%, 95%, 96%, 97%, 98% or 99% identical to the amino acid sequence as shown in SEQ ID NO: 1. Furthermore envisaged are NR2F6 orthologs which are at least 60%, 70%, 80%, 90%, 95%, 96%, 97%, 98% or 99% identical to the amino acid sequence as shown in SEQ ID NO: 2. In addition, the term “NR2F6 ortholog” comprises molecules which are at least 60%, more preferably at least 80% and most preferably at least 90% homologous to the polypeptide as shown in SEQ ID NO:2.

In order to determine whether a nucleic acid sequence has a certain degree of identity to a nucleic acid encoding NR2F6 orthologs, the skilled person can use means and methods well known in the art, e.g. alignments, either manually or by using computer programs such as those mentioned herein below in connection with the definition of the term “hybridization” and degrees of homology.

The term “hybridization” or “hybridizes” as used herein may relate to hybridizations under stringent or non-stringent conditions. If not further specified, the conditions are preferably non-stringent. Said hybridization conditions may be established according to conventional protocols described, e.g., in Sambrook, Russell “Molecular Cloning, A Laboratory Manual”, Cold Spring Harbor Laboratory, N.Y. (2001); Ausubel, “Current Protocols in Molecular Biology”, Green Publishing Associates and Wiley Interscience, N.Y. (1989), or Higgins and Hames (Eds.) “Nucleic acid hybridization, a practical approach” IRL Press Oxford, Washington D.C., (1985). The setting of conditions is well within the skill of the artisan and can be determined according to protocols described in the art. Thus, the detection of only specifically hybridizing sequences will usually require stringent hybridization and washing conditions such as 0.1×SSC, 0.1% SDS at 65° C. Non-stringent hybridization conditions for the detection of homologous or not exactly complementary sequences may be set at 6×SSC, 1% SDS at 65° C. As is well known, the length of the probe and the composition of the nucleic acid to be determined constitute further parameters of the hybridization conditions. Note that variations in the above conditions may be accomplished through the inclusion and/or substitution of alternate blocking reagents used to suppress background in hybridization experiments. Typical blocking reagents include Denhardt's reagent, BLOTTO, heparin, denatured salmon sperm DNA, and commercially available proprietary formulations. The inclusion of specific blocking reagents may require modification of the hybridization conditions described above, due to problems with compatibility. Hybridizing nucleic acid molecules also comprise fragments of the above described molecules. Such fragments may represent nucleic acid sequences which code for NR2F6 or a functional fragment thereof which have a length of at least 12 nucleotides, preferably at least 15, more preferably at least 18, more preferably of at least 21 nucleotides, more preferably at least 30 nucleotides, even more preferably at least 40 nucleotides and most preferably at least 60 nucleotides. Furthermore, nucleic acid molecules which hybridize with any of the aforementioned nucleic acid molecules also include complementary fragments, derivatives and allelic variants of these molecules. Additionally, a hybridization complex refers to a complex between two nucleic acid sequences by virtue of the formation of hydrogen bonds between complementary G and C bases and between complementary A and T bases; these hydrogen bonds may be further stabilized by base stacking interactions. The two complementary nucleic acid sequences hydrogen bond in an anti-parallel configuration. A hybridization complex may be formed in solution (e.g., Cot or Rot analysis) or between one nucleic acid sequence present in solution and another nucleic acid sequence immobilized on a solid support (e.g., membranes, filters, chips, pins or glass slides to which, e.g., cells have been fixed). The terms “complementary” or “complementarity” refer to the natural binding of polynucleotides under permissive salt and temperature conditions by base-pairing. For example, the sequence “A-G-T” binds to the complementary sequence “T-C-A”. Complementarity between two single-stranded molecules may be “partial”, in which only some of the nucleic acids bind, or it may be complete when total complementarity exists between single-stranded molecules. The degree of complementarity between nucleic acid strands has significant effects on the efficiency and strength of hybridization between nucleic acid strands. This is of particular importance in amplification reactions, which depend upon binding between nucleic acids strands.

The term “hybridizing sequences” preferably refers to sequences which display a sequence identity of at least 40%, preferably at least 50%, more preferably at least 60%, even more preferably at least 70%, particularly preferred at least 80%, more particularly preferred at least 90%, even more particularly preferred at least 95% and most preferably at least 97% identity with a nucleic acid sequence as described above encoding NR2F6 or a functional fragment thereof. Moreover, the term “hybridizing sequences” preferably refers to sequences encoding NR2F6 or a functional fragment thereof having a sequence identity of at least 40%, preferably at least 50%, more preferably at least 60%, even more preferably at least 70%, particularly preferred at least 80%, more particularly preferred at least 90%, even more particularly preferred at least 95% and most preferably at least 97% identity with an amino acid sequence of the NR2F6 sequences as described herein.

In accordance with the present invention, the term “identical” or “percent identity” in the context of two or more nucleic acid or amino acid sequences, refers to two or more sequences or subsequences that are the same, or that have a specified percentage of amino acid residues or nucleotides that are the same (e.g., 60% or 65% identity, preferably, 70-95% identity, more preferably at least 95% identity), when compared and aligned for maximum correspondence over a window of comparison, or over a designated region as measured using a sequence comparison algorithm as known in the art, or by manual alignment and visual inspection. Sequences having, for example, 60% to 95% or greater sequence identity are considered to be substantially identical. Such a definition also applies to the complement of a test sequence. Preferably the described identity exists over a region that is at least about 15 to 25 amino acids or nucleotides in length, more preferably, over a region that is about 50 to 100 amino acids or nucleotides in length. Those having skill in the art will know how to determine percent identity between/among sequences using, for example, algorithms such as those based on CLUSTALW computer program (Thompson Nucl. Acids Res. 2 (1994), 4673-4680) or FASTDB (Brutlag Comp. App. Biosci. 6 (1990), 237-245), as known in the art.

Although the FASTDB algorithm typically does not consider internal non-matching deletions or additions in sequences, i.e., gaps, in its calculation, this can be corrected manually to avoid an overestimation of the % identity. CLUSTALW, however, does take sequence gaps into account in its identity calculations. Also available to those having skill in this art are the BLAST and BLAST 2.0 algorithms (Altschul, (1997) Nucl. Acids Res. 25:3389-3402; Altschul (1993) J. Mol. Evol. 36:290-300; Altschul (1990) J. Mol. Biol. 215:403-410). The BLASTN program for nucleic acid sequences uses as defaults a word length (W) of 11, an expectation (E) of 10, M=5, N=4, and a comparison of both strands. For amino acid sequences, the BLASTP program uses as defaults a wordlength (W) of 3, and an expectation (E) of 10. The BLOSUM62 scoring matrix (Henikoff (1989) PNAS 89:10915) uses alignments (B) of 50, expectation (E) of 10, M=5, N=4, and a comparison of both strands.

Moreover, the present invention also relates to nucleic acid molecules whose sequence is being degenerate in comparison with the sequence of an above-described hybridizing molecule. When used in accordance with the present invention the term “being degenerate as a result of the genetic code” means that due to the redundancy of the genetic code different nucleotide sequences code for the same amino acid.

In order to determine whether an amino acid residue or nucleotide residue in a nucleic acid sequence corresponds to a certain position in the amino acid sequence or nucleotide sequence of e.g. SEQ ID NO: 2, the skilled person can use means and methods well-known in the art, e.g., alignments, either manually or by using computer programs such as those mentioned further down below in connection with the definition of the term “hybridization” and degrees of homology.

For example, BLAST 2.0, which stands for Basic Local Alignment Search Tool BLAST (Altschul (1997), loc. cit.; Altschul (1993), loc. cit.; Altschul (1990), loc. cit.), can be used to search for local sequence alignments. BLAST, as discussed above, produces alignments of both nucleotide and amino acid sequences to determine sequence similarity. Because of the local nature of the alignments, BLAST is especially useful in determining exact matches or in identifying similar sequences. The fundamental unit of BLAST algorithm output is the High-scoring Segment Pair (HSP). An HSP consists of two sequence fragments of arbitrary but equal lengths whose alignment is locally maximal and for which the alignment score meets or exceeds a threshold or cut-off score set by the user. The BLAST approach is to look for HSPs between a query sequence and a database sequence, to evaluate the statistical significance of any matches found, and to report only those matches which satisfy the user-selected threshold of significance. The parameter E establishes the statistically significant threshold for reporting database sequence matches. E is interpreted as the upper bound of the expected frequency of chance occurrence of an HSP (or set of HSPs) within the context of the entire database search. Any database sequence whose match satisfies E is reported in the program output.

Analogous computer techniques using BLAST (Altschul (1997), loc. cit.; Altschul (1993), loc. cit.; Altschul (1990), loc. cit.) are used to search for identical or related molecules in nucleotide databases such as GenBank or EMBL. This analysis is much faster than multiple membrane-based hybridizations. In addition, the sensitivity of the computer search can be modified to determine whether any particular match is categorized as exact or similar. The basis of the search is the product score which is defined as:

$$\frac{\% sequence identity \times \% maximum BLAST score}{100}$$

and it takes into account both the degree of similarity between two sequences and the length of the sequence match. For example, with a product score of 40, the match will be exact within a 1-2% error; and at 70, the match will be exact. Similar molecules are usually identified by selecting those which show product scores between 15 and 40, although lower scores may identify related molecules. Another example for a program capable of generating sequence alignments is the CLUSTALW computer program (Thompson (1994) Nucl. Acids Res. 2:4673-4680) or FASTDB (Brutlag (1990) Comp. App. Biosci. 6:237-245), as known in the art.

Based primarily on recent gene knockout approaches, a number of physiological functions of the COUP-TF family members have been described. NR2F1 knockout mice show glossopharyngeal ganglion and nerve defects and die shortly after birth due to suckling and swallowing problems (Qiu (1997) loc. cit.). Employing conditional knockouts, NR2F1 is critical for regulating axonal growth and the formation of commissural projections in the forebrain (Armentano (2006) loc. cit.). Additionally, a hypersensitivity to Notch-mediated suppression in NR2F1^{-/-} cochlea has been observed, which affects hair cell differentiation Tang (2006) Development 133:3683-3693). NR2F2 knockout mice die in utero due to angiogenesis and heart developmental problems (Pereira (1999) Genes & development 13:1037-1049). Analysis of NR2F2 knockout chimera revealed its critical role in maintaining vein identity, again by antagonizing Notch signalling (You (2005) loc. cit.). Conditional knockout mouse analysis revealed a role of NR2F2 during anterior posterior patterning of the stomach Takamoto (2005) loc. cit.). Additionally, NR2F2 were shown to exert a regulatory role in glucose homeostasis and insulin sensitivity (Bardoux (2005) Diabetes 54:1357-1363). In contrast to its family members NR2F1 and NR2F2, NR2F6 knockout mice are born at expected mendelian ratios (Warnecke (2005) loc. cit.).

A role for NR2F6 in regulating the immune response, however, has neither been described nor proposed in the prior art. Accordingly, it is most surprising that by inhibiting NR2F6 function, the immune response can be augmented. Accordingly, the present invention provides for the first medical use of antagonists/inhibitors of NR2F6 for the treatment of a disease related to an insufficient immune response.

The term “antagonist” or “inhibitor” as used herein is known in the art and relates to a compound/substance capable of fully or partially preventing or reducing the physiologic activity of (a) specific receptor(s). In the context of the present invention said antagonist, therefore, may prevent or reduce or inhibit or inactivate the physiological activity of a receptor such as NR2F6 upon binding of said compound/substance to said receptor. Binding of an “antagonist/inhibitor” to a given receptor, e.g. NR2F6, may compete with or prevent the binding of an endogenous activating ligand binding to said receptor. As used herein, accordingly, the term “antagonist” also encompasses competitive antagonists, (reversible) non-competitive antagonists or irreversible antagonist, as described, inter alia, in Mutschler, “Arzneimittelwirkungen” (1986), Wissenschaftliche Verlagsgesellschaft mbH, Stuttgart, Germany. In addition thereto, however, an “antagonist” or “inhibitor” of NR2F6 in the context of the present invention may also be capable of preventing the function of a given receptor, such as NR2F6, by preventing/reducing the expression of the nucleic acid molecule encoding for said receptor. Thus, an antagonist/inhibitor of NR2F6 may lead to a decreased expression level of NR2F6 (e.g. decreased level of NR2F6 mRNA, NR2F6 protein) which is reflected in an decreased activity of NR2F6. This decreased activity can be measured/detected by the herein described methods. An inhibitor of NR2F6 in the context of the present invention, accordingly, may also encompass transcriptional repressors of NR2F6 expression that are capable of reducing NR2F6 function. As described herein below in detail, the decreased expression and/or activity of NR2F6 by an antagonist/inhibitor of NR2F6 leads to an increased activity (and/or expression) of components of the NR2F6-dependent signaling pathway; in particular the activity of NF-AT and AP-1 is increased. NF-AT/AP-1 regulate transcription/expression of further “downstream” components of the NR2F6-dependent signaling pathway, such as IL-2, IL-17 and/or IFN-gamma. An increase in NF-AT/AP-1 activity results in an increased transcription of these “downstream” components (e.g. IL-2, IL-17 and/or IFN-gamma) which in turn leads to a stimulation or augmentation of an immune response. In sum, the herein described antagonist/inhibitor of NR2F6 will, accordingly, lead to a stimulation or augmentation of an immune response.

The terms “immune response” or “immune reaction” as used herein are known in the art and relate to the response/reaction of the immune system to an antigen. In case of an “immune response”, accordingly, immune cells are activated in such way that (a) specific function(s) of said immune cells is/are induced. Said “immune cells” may include, but are not limited to, B cells, T cells, neutrophils, eosinophils, basophils, mast cells, macrophages and dendritic cells. Said “(a) specific function(s) of activated immune cells” may include, but are not limited to, secretion of antibodies, presentation of antigen, proliferation of said immune cells, secretion of cytokines such as interleukin-2 (IL-2), interleukin-17 (IL-17) or IFNgamma (Interferon gamma), expression of regulatory-, activation- and/or adhesion molecules, and the ability to induce apoptosis and/or cytolysis. The term “antigen” as used herein refers to any substance capable of inducing an immune response. It is of note that an antigen typically is associated with a foreign substance (i.e. a “non-self antigen”). In addition thereto, however, an own body-derived substance (i.e. a “self antigen”) may also induce an immune response. As used herein, accordingly, the term “immune response” also encompasses “auto-immune responses” or “auto-immune reactions”. In a preferred embodiment, said antigen may be associated with a tumour cell.

An “insufficient immune response” is characterized by an unsatisfactory response/reaction of the immune system to an “antigen” or “antigenic stimulus”. Under normal, non-pathological conditions, immune responses are tightly regulated. Accordingly, the immune response is balanced in such way to ensure the elimination of an antigenic stimulus. In case of an “insufficient immune response” however, the immune response may be unable to achieve a sufficient elimination or sufficiently fast elimination of said antigenic stimulus. The term “disease related to an insufficient immune response”, accordingly, relates to any disease/disorder in which an insufficient immune response as defined herein above is etiological for, associated with, secondary to or the resultant of said disorder. An insufficient immune response may be determined by directly or indirectly measuring parameters which are indicative for the magnitude of the immune response/reaction to an antigen and comparing the outcome of said measurement raised in a to be tested subject with the outcome of the same test in a physiologically normal subject. Parameters indicative for the magnitude of the immune response/reaction may include, but are not limited to the presence/quantity of (specific) antibodies, presence/quantity of (specific) immune cells, the presence/quantity of (specific) cytokines and/or the presence/quantity of (specific) regulatory-, activation- and/or adhesion molecules.

For a disease to be related to an insufficient immune response, accordingly, said insufficient immune response may be detectable preceding, during or following said disease. In a preferred embodiment, the disease related to an insufficient immune response is primary immunodeficiency or acquired immunodeficiency. In another preferred embodiment, said primary immunodeficiency may be selected from the group consisting of, but not limited to severe combined immunodeficiency (SCID), common variable immunodeficiency (CVID or Hypogammaglobulinemia), X-linked agammaglobulinemia (Bruton type agammaglobulinemia), selective immunoglobulin A deficiency, IgG sub-class deficiency, hyper-IgM syndrome, complement deficiency, myeloperoxidase deficiency, leukocyte adhesion deficiency, chronic granulomatous disease, Ataxia telangiectasia, Wiskott-Aldrich syndrome, DiGeorge syndrome and Chédiak-Higashi syndrome. In another preferred embodiment, said acquired immunodeficiency is selected from the group consisting of, but not limited to multiple myeloma, chronic lymphatic leukaemia, drug-induced immunosuppression and acquired immune deficiency syndrome (AIDS). In yet another preferred embodiment, the disease related to an insufficient immune response is cancer. The cancer may also arise in context of/due to the acquired immunodeficiency described herein. In a preferred embodiment, the cancer is a solid tumour-induced cancer. Other cancers which arise due to an acquired immunodeficiency are multiple myeloma and chronic lymphatic leukaemia (chronic lymphatic leukaemia-induced cancer). In another preferred embodiment, said cancer may be selected from the group consisting of, but not limited to prostate cancer, breast cancer, lung cancer, colorectal cancer, bladder cancer, endometrial cancer, cutaneous melanoma, pancreatic cancer, ovarian cancer, neuroblastoma and glioblastoma.

Without being bound by theory, it is believed that the inhibitors/antagonists of NR2F6 interfere with the NR2F6-dependent signalling pathway as described herein below and exert thereby their effect on the stimulation of the immune response. It is, therefore, envisaged that NR2F6 inhibitors/antagonists (for example, and non-limiting NR2F6-specific siRNAs) can be used in the successful treatment of diseases related to an insufficient immune response.

As also demonstrated in the appended examples it has been surprisingly found herein that NR2F6 is a key element in a signalling cascade involved in the modulation of the immune response which is described in detail herein below.

As disclosed herein, NR2F6 acts as a novel transcriptional repressor in the adaptive immune system. In a simplified scheme, NR2F6 suppresses the immune response in the absence of a high-affinity antigen, or at least does not lead to an effective activation of the immune response triggered, for example, by a low-affinity antigen or by the release of interleukins. If a given antigen is present, the activity of NR2F6 is decreased, resulting in the activation (e.g. induction of expression as well as transactivation) of components of the NR2F6-dependent signalling pathway, such as NF-AT/AP-1. This will eventually lead to a stimulation of the immune response. It is apparent from the above that antagonizing/inhibiting NR2F6 will lead to an increased immune response and hence be beneficial in the treatment of diseases where stimulating the immune response is desirable.

The successful use of antagonists of NR2F6 disclosed herein is also illustrated by the herein described mouse model, i.e. the NR2F6^{-/-} deficient mice, wherein both alleles of the NR2F6 gene are knocked out, which reflects the action of NR2F6 antagonists/inhibitors on NR2F6 activity. The stimulation of the immune response in this mouse model is reflected in the following features observed in NR2F6 knockout mice, all of which are indicative of an increased immune response.

Gene ablation of NR2F6 causes increased CD4⁺ T and B cell activation responses (i.e. cell proliferation and interleukin secretion) as well as reduced cell death susceptibility in CD4⁺ T and B cells derived from 6 to 8 week old NR2F6 deficient mice. In particular, NR2F6 deficiency results in enhanced activation-induced proliferation and cytokine production of primary lymphocytes. As shown in the appended examples, the analysis of 10 month old NR2F6^{-/-} deficient mice revealed that spleens were much larger than those of wild type controls; see FIG. 5A. Consistently, 10 months old NR2F6^{-/-} deficient mice spontaneously developed exaggerated titters for IgG1.

As mentioned above, NR2F6 leads to an increase of the activity of at least one component of the NR2F6-dependent signalling pathway, such as NF-AT and/or AP-1 as key transcription factors. NF-AT and AP-1 upregulate the transcriptional expression of IL-2 (interleukin-2), IFNgamma and/or IL-17 (interleukin-17). Since the stimulation of interleukins plays a functional role in the regulation/stimulation of the immune response, the herein provided use of antagonists/inhibitors of NR2F6 activity opens a new field in the treatment of diseases related to an insufficient immune response. The stimulation of an otherwise insufficient immune response is particularly necessary and beneficial in diseases like cancer, AIDS and the like. Accordingly, the herein disclosed use of antagonists/inhibitors of NR2F6 offers relief and amelioration to patients suffering from diseases which are often difficult to cure.

NF-AT/AP-1 are key transcription factors, important both immediately after T cell receptor engagement but also during sustained transcriptional regulation of the T helper subset differentiation. NF-AT/AP-1 regulate the expression of the master transcription factors Gata3 (for the differentation into Th2 cells), RORgamma-t/RORalpha (for the differentation into Th17 cells) and Foxp3 (for the differentation into iTreg cells). Furthermore, the transcription factors NF-AT and AP-1 govern the transcription of most of the cytokines (for example IL-2, IL-17 and IFNgamma) secreted and thus the autocrine amplification as well as the effector functions of these different Th subsets.

As illustrated in the appended examples, NR2F6 acts as a direct repressor of NF-AT/AP-1 transactivation in CD4⁺ T cells, as observed by DNA binding analysis of NR2F6 proficient and deficient T cells (see FIGS. 4 & 18). In particular, NR2F6 prevents DNA-binding of NF-AT and/or AP-1 and this inhibits their activity to initiate the transcription/expression of e.g., IL-2, IL-17 and/or IFNgamma. Consequently, NR2F6 is disclosed herein as a key factor on the T cell stage that acts as a transcriptional repressor of NF-AT and/or AP-1-dependent target genes like, e.g. IL-2, IL-17 and/or IFN gamma. Thereby the immune response is suppressed.

Therefore, antagonists of NR2F6 are useful in stimulating the immune response. As shown in the appended examples, in the Electrophoretic Mobility Shift Assay (EMSA), TCR-induced activation of AP-1 and NF-AT (but not NF-kappaB) was significantly amplified by NR2F6 deficiency. NR2F6 also mediates differentiation of Treg cells, which are involved in the suppression of the immune system. Accordingly, inhibition of NR2F6 will prevent differentiation of Treg cells, resulting in a stimulation of the immune response.

As mentioned, the transcription factors NF-AT and AP-1 play an important role in the differentiation of various T helper cell images (i.e. Th17 or Treg). Upon exposure to antigen a signaling pathway is induced in naïve T cells (CD4⁺ T cells), which involves the NR2F6-dependent activation of NF-AT and AP-1. The activated transcription factors NF-AT and AP-1 in turn induce the expression of the master transcription factor of inducible regulatory T cells (Treg cells), FOXP3, which alters the expression of genes involved in the differentation of Treg cells.FOXP3 protein cooperates with NF-AT leading to the upregulation of CTLA-4 (Cytotoxic T-Lymphocyte Antigen 4) and CD25, two highly expressed surface markers of Treg cells; see Wu (2006) Cell, 126(2), 375-87.

In sum, it is clear from the above described NR2F6-dependent signalling pathway that antagonizing/inhibiting NR2F6 leads to an activation of components of said pathway (e.g. transcription factors NF-AT and/or AP-1) and the subsequent induction of the expression of “downstream” components, such as IL-2, IL-17 and IFN-gamma, and hence to a stimulated immune response. Also the differentiation and function of Treg cells (which are involved in the suppression of the immune response) is inhibited. It is therefore envisaged that antagonists/inhibitors of NR2F6 can be used in the successful treatment of diseases related to an insufficient immune response.

The terms “treatment”, “treating” and the like are used herein to generally mean obtaining a desired pharmacological and/or physiological effect. The effect may be prophylactic in terms of completely or partially preventing a disease or symptom thereof and/or may be therapeutic in terms of partially or completely curing a disease and/or adverse effect attributed to the disease. The term “treatment” as used herein covers any treatment of a disease in a subject and includes: (a) preventing a disease related to an insufficient immune response from occurring in a subject which may be predisposed to the disease; (b) inhibiting the disease, i.e. arresting its development; or (c) relieving the disease, i.e. causing regression of the disease.

A “patient” or “subject” for the purposes of the present invention includes both humans and other animals, particularly mammals, and other organisms. Thus, the methods are applicable to both human therapy and veterinary applications. In the preferred embodiment the patient is a mammal, and in the most preferred embodiment the patient is human.

The compounds capable of reducing NR2F6 function or (a) fragment(s) thereof, are expected to be very beneficial as agents in pharmaceutical settings disclosed herein and to be used for medical purposes, in particular, in the treatment of the diseases related to an insufficient immune response as described herein. Said antagonist/inhibitor of NR2F6 may be selected from the group consisting of small binding molecules, RNAi, anti-NR2F6 antisense molecules, intracellular binding-partners, aptamers or intramers.

Compounds which may function as specific an “antagonist” or “inhibitor” of NR2F6 may comprise small binding molecules such as small (organic) compounds or ligands for NR2F6. The term “small molecule” in the context of drug discovery is known in the art and relates to medical compounds having a molecular weight of less than 2,500 Daltons, preferably less than 1,000 Daltons, more preferably between 50 and 350 daltons. (Small) binding molecules comprise natural as well as synthetic compounds. The term “compound” in context of this invention comprises single substances or a plurality of substances. Said compound/binding molecules may be comprised in, for example, samples, e.g., cell extracts from, e.g., plants, animals or microorganisms. Furthermore, said compound(s) may be known in the art but hitherto not known to be capable of (negatively) influencing the activity NR2F6 or not known to be capable of influencing the expression of the nucleic acid molecule encoding for NR2F6, respectively. The plurality of compounds may be, e.g., added to a sample in vitro, to the culture medium or injected into the cell.

Yet it is also envisaged in the context of the present invention that compounds including, inter alia, peptides, proteins, nucleic acids including cDNA expression libraries, small organic compounds, ligands, PNAs and the like can be used as an antagonist of NR2F6 function. Said compounds can also be functional derivatives or analogues. Methods for the preparation of chemical derivatives and analogues are well known to those skilled in the art and are described in, for example, Beilstein, “Handbook of Organic Chemistry”, Springer Edition New York, or in “Organic Synthesis”, Wiley, New York. Furthermore, said derivatives and analogues can be tested for their effects, i.e. their antagonistic effects of NR2F6 function according to methods known in the art. Furthermore, peptidomimetics and/or computer aided design of appropriate antagonists or inhibitors of NR2F6 can be used. Appropriate computer systems for the computer aided design of, e.g., proteins and peptides are described in the prior art, for example, in Berry (1994) Biochem. Soc. Trans. 22:1033-1036; Wodak (1987), Ann. N.Y. Acad. Sci. 501:1-13; Pabo (1986), Biochemistry 25:5987-5991. The results obtained from the above-described computer analysis can be used in combination with the method of the invention for, e.g., optimizing known compounds, substances or molecules. Appropriate compounds can also be identified by the synthesis of peptidomimetic combinatorial libraries through successive chemical modification and testing the resulting compounds, e.g., according to the methods described herein. Methods for the generation and use of peptidomimetic combinatorial libraries are described in the prior art, for example in Ostresh (1996) Methods in Enzymology 267:220-234 and Dorner (1996) Bioorg. Med. Chem. 4:709-715. Furthermore, the three-dimensional and/or crystallographic structure of antagonists of NR2F6 can be used for the design of (peptidomimetic) antagonists of NR2F6 (Rose (1996) Biochemistry 35:12933-12944; Rutenber (1996) Bioorg. Med. Chem. 4:1545-1558).

The RNAi-approach is also envisaged in context of this invention for use in the preparation of a pharmaceutical composition for the treatment of diseases/disorders related to an insufficient immune response as disclosed herein.

The term “RNA interference” or “inhibiting RNA” (RNAi/iRNA) describes the use of double-stranded RNA to target specific mRNAs for degradation, thereby silencing their expression. Preferred inhibiting RNA molecules may be selected from the group consisting of double-stranded RNA (dsRNA), RNAi, siRNA, shRNA and stRNA. dsRNA matching a gene sequence is synthesized in vitro and introduced into a cell. The dsRNA may also be introduced into a cell in form of a vector expressing a target gene sequence in sense and antisense orientation, for example in form of a hairpin mRNA. The sense and antisense sequences may also be expressed from separate vectors, whereby the individual antisense and sense molecules form double-stranded RNA upon their expression. It is known in the art that in some occasions the expression of a sequence in sense orientation or even of a promoter sequence suffices to give rise to dsRNA and subsequently to siRNA due to internal amplification mechanisms in a cell. Accordingly, all means and methods which result in a decrease in activity (which may be reflected in a lower expression of NR2F6), in particular by taking advantage of NR2F6-specific siRNAs (i.e. siRNAs that target specifically NR2F6 mRNA or a functional fragment thereof) are to be used in accordance with the present invention. For example sense constructs, antisense constructs, hairpin constructs, sense and antisense molecules and combinations thereof can be used to generate/introduce these siRNAs. The dsRNA feeds into a natural, but only partially understood process including the highly conserved nuclease dicer which cleaves dsRNA precursor molecules into short interfering RNAs (siRNAs). The generation and preparation of siRNA(s) as well as the method for inhibiting the expression of a target gene is, inter alia, described in WO 02/055693, Wei (2000) Dev. Biol. 15:239-255; La Count (2000) Biochem. Paras. 111:67-76; Baker (2000) Curr. Biol. 10:1071-1074; Svoboda (2000) Development 127:4147-4156 or Marie (2000) Curr. Biol. 10:289-292. These siRNAs built then the sequence specific part of an RNA-induced silencing complex (RISC), a multicomplex nuclease that destroys messenger RNAs homologous to the silencing trigger). Elbashir (2001) EMBO J. 20:6877-6888 showed that duplexes of 21 nucleotide RNAs may be used in cell culture to interfere with gene expression in mammalian cells. It is already known that RNAi is mediated very efficiently by siRNA in mammalian cells but the generation of stable cell lines or non-human transgenic animals was limited. However, new generations of vectors may be employed in order to stably express, e.g. short hairpin RNAs (shRNAs). Stable expression of siRNAs in Mammalian Cells is inter alia shown in Brummelkamp (2002) Science 296:550-553. Also Paul (2002) Nat. Biotechnol. 20:505-508 documented the effective expression of small interfering RNA in human cells. RNA interference by expression of short-interfering RNAs and hairpin RNAs in mammalian cells was also shown by Yu (2002) PNAS 99:6047-6052. The shRNA approach for gene silencing is well known in the art and may comprise the use of st (small temporal) RNAs; see, inter alia, Paddison (2002) Genes Dev. 16:948-958. These approaches may be vector-based, e.g. the pSUPER vector, or RNA polIII vectors may be employed as illustrated, inter alia, in Yu (2002), loc. cit.; Miyagishi (2002), loc. cit. or Brummelkamp (2002), loc. cit. It is envisaged that the regulatory sequences of the present invention are used in similar fashion as the systems based on pSUPER or RNA polIII vectors.

Methods to deduce and construct siRNAs are known in the art and are described in Elbashir (2002) Methods 26:199-213, at the internet web sites of commercial vendors of siRNA, e.g. Qiagen GmbH (https://www1.qiagen.com/GeneGlobe/Default.aspx); Dharmacon (www.dharmacon.com); Xeragon Inc. (http://www.dharmacon.com/Default.aspx), and Ambion (www.ambion.com), or at the web site of the research group of Tom Tuschl (http://www.rockefeller.edu/labheads/tuschl/sirna.html). In addition, programs are available online to deduce siRNAs from a given mRNA sequence (e.g. http://www.ambion.com/techlib/misc/siRNA_finder.html or http://katandin.cshl.org:9331/RNAi/html/mai.html). These were also used to deduce the siRNA molecules listed below (siRNAi 1 to 4; SEQ ID NO: 19 to 26). Uridine residues in the 2-nt 3′ overhang can be replaced by 2′ deoxythymidine without loss of activity, which significantly reduces costs of RNA synthesis and may also enhance resistance of siRNA duplexes when applied to mammalian cells (Elbashir (2001) loc. cit). The siRNAs may also be synthesized enzymatically using T7 or other RNA polymerases (Donze (2002) Nucleic Acids Res 30:e46). Short RNA duplexes that mediate effective RNA interference (esiRNA) may also be produced by hydrolysis with Escherichia coli RNase III (Yang (2002) PNAS 99:9942-9947). Furthermore, expression vectors have been developed to express double stranded siRNAs connected by small hairpin RNA loops in eukaryotic cells (e.g. (Brummelkamp (2002) Science 296:550-553). All of these constructs may be developed with the help of the programs named above. In addition, commercially available sequence prediction tools incorporated in sequence analysis programs or sold separately, e.g. the siRNA Design Tool offered by www.oligoEngine.com (Seattle, Wash.) may be used for siRNA sequence prediction.

Accordingly, specific interfering RNAs can be used in accordance with the present invention as antagonists (inhibitors) of NR2F6 (expression and/or function). These siRNAs are formed by an antisense and a sense strand, whereby the antisense/sense strand preferably comprises at least 10, more preferably at least 12, more preferably at least 14, more preferably at least 16, more preferably at least 18, more preferably at least 19, 20, 21 or 22 nucleotides. The use of the following siRNAs is particularly preferred in context of the present invention:

siRNAi 1 (a): GUGGAAAGCAUUACGGCGUUU (SEQ ID No: 19) and ACGCCGUAAUGCUUUCCACUU (SEQ ID No: 20) (corresponding to the Dharmacon SMARTpool duplex [9], order code J-045088-09);
siRNAi 2 (b): AGGUGGAUGCUGCGGAGUAUU (SEQ ID No: 21) and UACUCCGCAGCAUCCACCUUU (SEQ ID No: 22) (corresponding to the Dharmacon SMARTpool duplex [10], order code J-045088-10);
siRNAi 3 (c): GCAUCGACAACGUGUGCGAUU (SEQ ID No: 23) and UCGCACACGUUGUCGAUGCUU (SEQ ID No: 24) (corresponding to the Dharmacon SMARTpool duplex [11], order code J-045088-11); and
siRNAi 4 (d): GGCAAGACACCCAUCGAGAUU (SEQ ID No: 25); and UCUCGAUGGGUGUCUUGCCUU (SEQ ID No: 26) (corresponding to the Dharmacon SMARTpool duplex [12], order code J-045088-12).

It is preferred herein that the 5′ end of the siRNAs (in particular of SEQ ID NOs. 20, 22, 24, and 26) is phosphorylated. If the 5′ end is phosphorylated, the respective sequences can also be depicted as follows: 5′-PACGCCGUAAUGCUUUCCACUU (SEQ ID No: 20), 5′-PUACUCCGCAGCAUCCACCUUU (SEQ ID No: 22), 5′-PUCGCACACGUUGUCGAUGCUU (SEQ ID No: 24) and 5′-PUCUCGAUGGGUGUCUUGCCUU (SEQ ID No: 26), wherein “5′-P” reflects the phosphorylated 5′ end.

As mentioned above, methods for preparing siRNAs to be used in accordance with the present invention are well known in the art. Based on the teaching provided herein, a skilled person in the art is easily in the position not only to prepare such siRNAs but also to assess whether a siRNA is capable of antagonizing/inhibiting NR2F6. It is envisaged herein that the above described siRNAs lead to a degradation of NR2F6 mRNA and thus to a decreased protein level of NR2F6.

In other words, siRNAs lead to a pronounced decrease in mRNA and/or protein levels of NR2F6 (i.e. to a reduced expression of NR2F6). This decrease in expression may be reflected in a decreased activity of NR2F6. For example, NR2F6-specific siRNAs may lead to a decreased capacity of NR2F6 to inhibit the activity of NF-AT/AP-1 (and, subsequently, also the expression of IL-2, IL-17, IFNgamma and other components of the NR2F6-dependent signaling pathway). Hence, the use of potent antagonists/inhibitors of NR2F6 (such as the herein described siRNAs) will lead to a higher activity of NF-AT/AP-1 and thus higher expression of NF-AT/AP-1 dependent target genes (such as IL-2, IL-17 and IFNgamma as well as other components of the NR2F6-dependent signaling pathway) which will in turn result in an enhanced/augmented immune response. An exemplary transfection of CD4⁺ T cells with NR2F6-specific siRNAs is also shown in the appended example. As demonstrated therein, the siRNA transfection leads to a pronounced decrease of the expression of NR2F6 of at least 95%. Furthermore, a decrease of the NR2F6 activity/expression leads to an increase of the cytokine response level; see FIG. 16.

As used herein the term “small interfering RNA” (siRNA), sometimes known as short interfering RNA or silencing RNA, refers to a class of generally short and double-stranded RNA molecules that play a variety of roles in biology and, to an increasing extent, in treatment of a variety of diseases and conditions. As mentioned above, siRNAs are involved in the RNA interference (RNAi) pathway where the siRNA interferes with the expression of a specific gene (see, e.g. Zamore Nat Struct Biol 2001, 8(9):746-50; Tuschl T. CHEMBIOCHEM. 2001, 2:239-245; Scherr and Eder, Cell Cycle. 2007 February; 6(4):444-9; Leung and Whittaker, Pharmacol Ther. 2005 August; 107(2):222-39; de Fougerolles et al., Nat. Rev. Drug Discov. 2007, 6: 443-453).

Such siRNAs are generally 18-27 nt long, generally comprising a short (usually 19-21-nt) double-strand of RNA (dsRNA) with or without 2-nt 3′ overhangs on either end. Each strand can have a 5′ phosphate group and a 3′ hydroxyl (—OH) group or the phosphate group can be absent on one or both strands. This structure is the result of processing by dicer, an enzyme that converts either long dsRNAs or small hairpin RNAs into siRNAs.

siRNAs can also be exogenously (artificially) introduced into cells by various transfection methods to bring about the specific knockdown of a gene of interest. In this context, other structures than those described above are also envisaged, provided they are capable of interfering with gene expression. Preferably, the double-stranded part has a length of about 12 to about 50 base pairs, more preferably 16 to 30, more preferably 18 to 25, more preferably 19 to 21 in length. Most preferably, the double-stranded part has a length of 19 base pairs. The siRNA of the invention may either have overhanging sequences of up to 10 bases, preferably not more than 5 bases in length at either end or at one end, or may be blunt-ended. Also preferred is that the complementarity to the target gene extends over the entire length of the double-stranded part. The region which is complementary to the target gene is at least 12 bases, preferably at least 15, 16, 17, 18, 19, 20, 21, 22, 23 or more bases in length. The siRNA of the invention may be fully complementary to the target gene. Alternatively, the siRNA may comprise up to 5%, 10%, 20% or 30% mismatches to the target gene. Furthermore, siRNAs and also antisense RNAs can be chemically modified e.g. on the backbone including the sugar residues. Preferred modifications of the siRNA molecules of the invention include linkers connecting the two strands of the siRNA molecule. Chemical modifications serve inter alia to improve the pharmacological properties of siRNAs and antisense RNAs such as in vivo stability and/or delivery to the target site within an organism. The skilled person is aware of such modified siRNAs as well as of means and methods of obtaining them, see, for example, Zhang et al., Curr Top Med. Chem. 2006; 6(9):893-900; Manoharan, Curr Opin Chem. Biol. 2004 December; 8(6):570-9.

Essentially any gene of which the sequence is known can thus be targeted based on sequence complementarity with an appropriately tailored siRNA. This has made siRNAs an important tool for gene function and drug target validation studies as well as for therapeutic intervention which is envisaged here. The siRNAs disclosed herein are capable of reducing or blocking the expression of NR2F6.

In a further aspect, it is envisaged that antisense molecules inhibit the expression or function of NR2F6, in particular of human NR2F6 and interact with NR2F6 as expressed by the coding regions, mRNAs/cDNAs as defined herein above as well as with NR2F6 as expressed by isoforms and variants of said NR2F6. Said isoforms or variants may, inter alia, comprise allelic variants or splice variants. Furthermore, it is also envisaged that the antisense molecules to be used in accordance with the present invention against NR2F6 expression or function interfere specifically with regulatory sequences of NR2F6 as defined herein below.

The term “variant” means in this context that the NR2F6 nucleotide sequence and the encoded NR2F6 amino acid sequence, respectively, differs from the distinct sequences available under the above-identified GenBank Accession numbers, by mutations, e.g. deletion, additions, substitutions, inversions etc.

Therefore, the antisense-molecule to be employed in accordance with the present invention specifically interacts with/hybridizes to one or more nucleic acid molecules encoding NR2F6. Preferably said nucleic acid molecule is RNA, i.e. pre m-RNA or mRNA. The term “specifically interacts with/hybridizes to one or more nucleic acid molecules encoding NR2F6” relates, in context of this invention, to antisense molecules which are capable of interfering with the expression of NR2F6. Yet, highly mutated anti-NR2F6 antisense constructs, which are not capable of hybridizing to or specifically interacting with NR2F6-coding nucleic acid molecules are not to be employed in the context of the present invention. The person skilled in the art can easily deduce whether an antisense construct specifically interacts with/hybridizes to NR2F6 encoding sequences. These tests comprise, but are not limited to hybridization assays, RNAse protection assays, Northern Blots, North-western blots, nuclear magnetic resonance and fluorescence binding assays, dot blots, micro- and macroarrays and quantitative PCR. In addition, such a screening may not be restricted to NR2F6 mRNA molecules, but may also include NR2F6 mRNA/protein (RNP) complexes (Hermann (2000) Angew Chem Int Ed Engl 39:1890-1904; DeJong (2002) Curr Trop Med Chem 2:289-302). Furthermore, functional tests including Western blots, immunohistochemistry, immunoprecipitation assay, and bioassays based on NR2F6-responsive promoters are envisaged for testing whether a particular antisense construct is capable of specifically interacting with/hybridizing to the NR2F6 encoding nucleic acid molecules.

The term “antisense-molecule” as used herein comprises in particular antisense oligonucleotides. Said antisense oligonucleotides may also comprise modified nucleotides as well as modified internucleoside-linkage, as, inter alia, described in U.S. Pat. No. 6,159,697.

Most preferably, the antisense oligonucleotides of the present invention comprise at least 8, more preferably at least 10, more preferably at least 12, more preferably at least 14, more preferably at least 16 nucleotides. The deduction as well as the preparation of antisense molecules is very well known in the art. The deduction of antisense molecules is, inter alia, described in Smith, 2000. Usual methods are “gene walking”, Rnase H mapping, RNase L mapping (Leaman (1999) Meth Enzymol 18:252-265), combinatorial oligonucleotide arrays on solid support, determination of secondary structure analysis by computational methods (Walton (2000) Biotechnol Bioeng, 65:1-9), aptamer oligonucleotides targeted to structured nucleic acids (aptastruc), thetered oligonucleotide probes, foldback triplex-forming oligonucleotides (FTFOs) (Kandimalla (1994) Gene 149:115-121) and selection of sequences with minimized non-specific binding (Han (1994) Antisense Res Dev 4:53-65).

Preferably, the antisense molecules of the present invention are stabilized against degradation. Such stabilization methods are known in the art and, inter alia, described in U.S. Pat. No. 6,159,697. Further methods described to protect oligonucleotides from degradation include oligonucleotides bridged by linkers (Vorobjev (2001) Antisense Nucleic Acid Drug Dev, 11:77-85), minimally modified molecules according to cell nuclease activity (Samani (2001) Antisense Nucleic Acid Drug Dev, 11:129-136), 2′O-DMAOE oligonucleotides (Prakash (2001) Nucleosides Nucleotides Nucleic Acids 20:829-832), 3′5′-Dipeptidyl oligonucleotides (Schwope (1999) J Org Chem 64:4749-4761), 3′ methylene thymidine and 5-methyluridine/cytidine h-phosphonates and phosphonamidites (An (2001) J Org Chem, 66:2789-2801), as well as anionic liposome (De Oliveira (2000) Life Sci 67:1625-1637) or ionizable aminolipid (Semple (2001) Biochim Biophys Acta, 10:152-166) encapsulation.

In addition thereto, the antagonist/inhibitor of NR2F6 expression or function may also comprise intracellular binding partners of NR2F6. As used herein, the term “intracellular binding partner” relates to intracellular molecules capable of preventing or reducing NR2F6 activity. Such intracellular binding partners of NR2F6, inter alia, may relate to endogenous inhibitor/repressor proteins of NR2F6. In another embodiment of the invention the intracellular binding partner is an intracellular antibody. Intracellular antibodies are known in the art and can be used to modulate or inhibit the functional activity of the target molecule. This therapeutic approach is based on intracellular expression of recombinant antibody fragments, either Fab or single chain Fv, targeted to the desired cell compartment using appropriate targeting sequences (Teillaud (1999) Pathol Biol 47:771-775).

As mentioned herein above, the antagonist/inhibitor of NR2F6 expression or function may also comprise an aptamer. In the context of the present invention, the term “aptamer” comprises nucleic acids such as RNA, ssDNA (ss=single stranded), modified RNA, modified ssDNA or PNAs which bind a plurality of target sequences having a high specificity and affinity. Aptamers are well known in the art and, inter alia, described in Famulok (1998) Curr. Op. Chem. Biol. 2:320-327. The preparation of aptamers is well known in the art and may involve, inter alia, the use of combinatorial RNA libraries to identify binding sites (Gold (1995) Ann. Rev. Biochem. 64:763-797).

Accordingly, aptamers are oligonucleotides derived from an in vitro evolution process called SELEX (systematic evolution of ligands by exponential enrichment). Pools of randomized RNA or single stranded DNA sequences are selected against certain targets. The sequences of tighter binding with the targets are isolated and amplified. The selection is repeated using the enriched pool derived from the first round selection. Several rounds of this process lead to winning sequences that are called “aptamers”. Aptamers have been evolved to bind proteins which are associated with a number of disease states. Using this method, many powerful antagonists of such proteins can be found. In order for these antagonists to work in animal models of disease and in humans, it is normally necessary to modify the aptamers. First of all, sugar modifications of nucleoside triphosphates are necessary to render the resulting aptamers resistant to nucleases found in serum. Changing the 2′OH groups of ribose to 2′F or 2′NH2 groups yields aptamers which are long lived in blood. The relatively low molecular weight of aptamers (8000-12000) leads to rapid clearance from the blood. Aptamers can be kept in the circulation from hours to days by conjugating them to higher molecular weight vehicles. When modified, conjugated aptamers are injected into animals, they inhibit physiological functions known to be associated with their target proteins. Aptamers may be applied systemically in animals and humans to treat organ specific diseases (Ostendorf (2001) J Am Soc Nephrol. 12:909-918). The first aptamer that has proceeded to phase I clinical studies is NX-1838, an injectable angiogenesis inhibitor that can be potentially used to treat macular degeneration-induced blindness. (Sun (2000) Curr Opin Mol Ther 2:100-105). Cytoplasmatic expression of aptamers (“intramers”) may be used to bind intracellular targets (Blind (1999) PNAS 96:3606-3610; Mayer (2001) PNAS 98:4961-4965). Said intramers are also envisaged to be employed in context of this invention.

As used herein, the term “nucleic acid sequence” relates to the sequence of bases comprising purine- and pyrimidine bases which are comprised by nucleic acid molecules, whereby said bases represent the primary structure of a nucleic acid molecule. Nucleic acid sequences include DNA, cDNA, genomic DNA, RNA, synthetic forms and mixed polymers, both sense and antisense strands, or may contain non-natural or derivatized nucleotide bases, as will be readily appreciated by those skilled in the art. Preferably, the term “NR2F6” when used in the context of expressing NR2F6 refers to the nucleic acid molecule encoding NR2F6 protein, or a functional fragment thereof. Exemplary nucleic acid sequences are known in the art and also disclosed herein.

As used herein, the term “polypeptide” relates to a peptide, a protein, or a polypeptide which encompasses amino acid chains of a given length, wherein the amino acid residues are linked by covalent peptide bonds. However, peptidomimetics of such proteins/polypeptides wherein amino acid(s) and/or peptide bond(s) have been replaced by functional analogs are also encompassed by the invention as well as other than the 20 gene-encoded amino acids, such as selenocysteine. Peptides, oligopeptides and proteins may be termed polypeptides. The terms polypeptide and protein are often used interchangeably herein. The term polypeptide also refers to, and does not exclude, modifications of the polypeptide, e.g., glycosylation, acetylation, phosphorylation and the like. Such modifications are well described in basic texts and in more detailed monographs, as well as in a voluminous research literature. Preferably, the term “NR2F6”, particularly when used in context of “activity of NR2F6”, refers to the protein/polypeptide having the specific NR2F6 activity as disclosed herein.

As used herein, a “functional fragment” of a protein which displays a specific biological activity relates to fragments of said protein having a sufficient length to display said activity. Accordingly, a functional fragment of a protein showing e.g. a specific (enzymatic) activity may relate to a polypeptide which corresponds to a fragment of said protein which is still capable of showing said (enzymatic) activity. For example, a functional fragment of NR2F6 in the context of the ligand binding activity of NR2F6 may correspond to the ligand-binding domain of NR2F6 as defined herein below. Methods for determining whether a certain fragment of a protein is a functional fragment are known in the art. For example, test for determining whether a fragment of NR2F6 is still capable of binding a ligand are described herein below. Preferably, a functional fragment of NR2F6 has substantially the same biological activity as NR2F6 itself. Furthermore, a person skilled in the art will be aware that the (biological) activity as described herein often correlates with the expression level, preferably the protein or mRNA level. The term “expression” as used herein refers to the expression of a nucleic acid molecule encoding a polypeptide/protein, whereas “activity” refers to the activity of said polypeptide/protein, which can be determined as outlined herein. The explanations given herein above and below in respect of the activity of “NR2F6” and “components of the NR2F6-dependent signalling cascade”, respectively, also apply, mutatis mutandis, to (a) “functional fragment(s) of NR2F6” and to (a) “functional fragment(s) of a component of the NR2F6-dependent signalling cascade”. In other words, a “functional fragment of NR2F6” has essentially the same activity as NR2F6 as defined herein and a “functional fragment of a component of the NR2F6-dependent signalling cascade” has, correspondingly, essentially the same activity as said “component(s) of the NR2F6-dependent signalling cascade” as defined herein. Accordingly, also inhibitors/antagonists of functional fragments of NR2F6 are disclosed and provided herein. As mentioned, methods/assays for determining the activity of “NR2F6”, “components of the NR2F6-dependent signalling cascade”, “functional fragment of NR2F6” and “functional fragment of components of the NR2F6-dependent signalling cascade” are well known in the art and also described herein above and below. Preferably, the functional fragment has at least 60%, more preferably at least 70%, 75%, 80%, 85%, 90% and even more preferably at least 95% or 99% of NR2F6 and the component(s) of the NR2F6-dependent signalling cascade, respectively.

NR2F6 antagonists/inhibitors of NR2F6 function may be deduced by methods in the art. Such methods are described herein and, inter alia, may comprise, but are not limited to methods where a collection of substances is tested for interaction with NR2F6 or with (a) fragment(s) thereof and where substances which test positive for interaction in a corresponding readout system are further tested in vivo, in vitro or in silico for their inhibiting effects on NR2F6 expression or function.

Said “test for NR2F6 interaction” of the above described method may be carried out by specific immunological, molecular biological and/or biochemical assays which are well known in the art and which comprise, e.g., homogenous and heterogenous assays as described herein below. The natural endogenous ligand(s) of NR2F6 remain(s) to be identified. Yet, NR2F6 ligands capable of inhibiting NR2F6 function may be identified by screening large compound libraries based on their capacity to interact with the NR2F6 protein. In a preferred embodiment, such antagonists or inhibitors of NR2F6 function are capable of binding the ligand binding domain of NR2F6.

The person skilled in the art is readily capable of identifying the ligand-binding domain of the NR2F6 protein. Accordingly, the ligand-binding domain of human wild type NR2F6 (drugable domain) may be encoded by the following nucleic acid sequence:

| (SEQ ID NO: 3) | |
|---|---|
| 743 | cggctgc tcttcagcac cgtggagtgg gcgcgccacg |
| 781 | cgcccttctt ccccgagctg ccggtggccg accaggtggc gctgctgcgc ctgagctgga |
| 841 | gcgagctctt cgtgctgaac gcggcgcagg cggcgctgcc cctgcacacg gcgccgctac |
| 901 | tggccgccgc cggcctccac gccgcgccta tggccgccga gcgcgccgtg gctttcatgg |
| 961 | accaggtgcg cgccttccag gagcaggtgg acaagctggg ccgcctgcag gtcgactcgg |
| 1021 | ccgagtatgg ctgcctcaag gccatcgcgc tcttcacgcc cgacgcctgt ggcctctcag |
| 1081 | acccggccca cgttgagagc ctgcaggaga aggcgcaggt ggccctcacc gagtatgtgc |
| 1141 | gggcgcagta cccgtcccag ccccagcgct tcgggcgcct gctgctgcgg ctccccgccc |
| 1201 | tgcgcgcggt ccctgcctcc ctcatctccc agctgttctt catgcgcctg gtggggaaga |
| 1261 | cgcccattga gacactgatc agagacatgc tgctgtcggg gagtaccttc aactggccct |
| 1321 | acggctcggg ccagtgacca tgacggggcc acgtgtgctg tggccaggcc tgcagacaga |
| 1381 | cctcaaggga cagggaatgc tgaggcctcg aggggcctcc cggggcccag gactctggct |
| 1441 | tctctcctca gacttctatt ttttaaagac tgtgaaatgt ttgtcttttc tgttttttaa |
| 1501 | atgatcatga aaccaaaaag agactgatca tccaggcctc agcctcatcc tccccaggac |
| 1561 | ccctgtccag gatggagggt ccaatcctag gacagccttg ttcctcagca cccctagcat |
| 1621 | gaacttgtgg gatggtgggg ttggcttccc tggcatgatg gacaaaggcc tggcgtcggc |
| 1681 | cagaggggct gctccagtgg gcaggggtag ctagcgtgtg ccaggcagat cctctggaca |
| 1741 | cgtaacctat gtcagacact acatgatgac tcaaggccaa taataaagac atttcctacc |
| 1801 | tgca |

which corresponds to the following amino acid sequence:

(SEQ ID NO: 4)
ARLLFSTVEWARHAPFFPELPVADQVALLRLSWSELFVLNAAQAALPL
HTAPLLAAAGLHAAPMAAERAVAFMDQVRAFQEQVDKLGRLQVDSAEY
GCLKAIALFTPDACGLSDPAHVESLQEKAQVALTEYVRAQYPSQPQRF
GRLLLRLPALRAVPASLISQLFFMRLVGKTPIETLIRDMLLSGSTFNW
PYGSGQ

It can be easily deduced from the human wild-type NR2F6 nucleic acid and amino acid sequences disclosed herein above (SEQ ID NOs: 1 and 2) that the ligand-binding domain of human wild-type NR2F6 (nucleic acid and amino acid sequence shown in SEQ ID NOs: 3 and 4, respectively) corresponds to the N-terminal part of the full length NR2F6 protein. The ligand-binding domain of wild-type NR2F6 is, accordingly, encoded by a nucleic acid sequence (as depicted in SEQ ID NO: 3) corresponding to nucleotides 743 to 1804 of the full length wild-type NR2F6 nucleic acid sequence.

Besides molecules capable of binding to NRF6, antagonists or inhibitors of NR2F6 function may be capable of preventing/reducing the expression of the nucleic acid molecule encoding the NR2F6 protein. The skilled person is readily capable of identifying regulatory sequences (such as promoter sequences, enhancer sequences, replication origins and other regulatory elements) of NR2F6 expression e.g. by using in silico gene prediction methods and experimental validation of functional sites (Elnitski (2006) Genome Res 16:1455-64).

In a further aspect, the present invention relates to a pharmaceutical composition comprising the antagonist/inhibitor of NR2F6 as described herein, optionally further comprising a pharmaceutical carrier. The (pharmaceutical) compositions of the invention may be in solid or liquid form and may be, inter alia, in a form of (a) powder(s), (a) tablet(s), (a) solution(s) or (an) aerosol(s). Furthermore, it is envisaged that the medicament of the invention might comprise further biologically active agents, depending on the intended use of the pharmaceutical composition.

Administration of the suitable (pharmaceutical) compositions may be effected by different ways, e.g., by parenteral, subcutaneous, intraperitoneal, topical, intrabronchial, intrapulmonary and intranasal administration and, if desired for local treatment, intralesional administration. Parenteral administrations include intraperitoneal, intramuscular, intradermal, subcutaneous intravenous or intraarterial administration. The compositions of the invention may also be administered directly to the target site, e.g., by biolistic delivery to an external or internal target site, like a specifically effected organ.

Examples of suitable pharmaceutical carriers, excipients and/or diluents are well known in the art and include phosphate buffered saline solutions, water, emulsions, such as oil/water emulsions, various types of wetting agents, sterile solutions etc. Compositions comprising such carriers can be formulated by well known conventional methods. Suitable carriers may comprise any material which, when combined with the biologically active protein of the invention, retains the biological activity of the comprised antagonist/inhibitor of NR2F6 (see Remington's Pharmaceutical Sciences (1980) 16th edition, Osol, A. Ed). Preparations for parenteral administration may include sterile aqueous or non-aqueous solutions, suspensions, and emulsions). Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles may include sodium chloride solution, Ringer's dextrose, dextrose and sodium chloride, lactated Ringer's, or fixed oils. Intravenous vehicles may include fluid and nutrient replenishes, electrolyte replenishers (such as those based on Ringer's dextrose), and the like. Preservatives and other additives may also be present including, for example, antimicrobials, anti-oxidants, chelating agents, and inert gases and the like. In addition, the pharmaceutical composition of the present invention might comprise proteinaceous carriers, like, e.g., serum albumin or immunoglobulin, preferably of human origin.

These pharmaceutical compositions can be administered to the subject at a suitable dose. The dosage regimen will be determined by the attending physician and clinical factors. As is well known in the medical arts, dosages for any one patient depend upon many factors, including the patient's size, body surface area, age, the particular compound to be administered, sex, time and route of administration, general health, and other drugs being administered concurrently. Pharmaceutically active matter may be present in amounts between 1 μg and 20 mg/kg body weight per dose, e.g. between 0.1 mg to 10 mg/kg body weight, e.g. between 0.5 mg to 5 mg/kg body weight. If the regimen is a continuous infusion, it should also be in the range of 1 μg to 10 mg per kilogram of body weight per minute. Yet, doses below or above the indicated exemplary ranges also are envisioned, especially considering the aforementioned factors.

Furthermore, it is envisaged that the pharmaceutical composition of the invention might comprise further biologically active agents, depending on the intended use of the pharmaceutical composition. These further biologically active agents may be e.g. antibodies, antibody fragments, hormones, growth factors, enzymes, binding molecules, cytokines, chemokines, nucleic acid molecules and drugs. In a preferred embodiment, the pharmaceutical composition of the present invention is to be co-administered with other known immunoaugmenting drugs or treatments. In another preferred embodiment, the pharmaceutical composition of the invention may be co-administered with an anti-tumour chemotherapeutic agent, dendritic cell (DC)-based tumor vaccines and/or anti-tumour radiation when said pharmaceutical composition is used for treating cancer. Said anti-tumour chemotherapeutic agent may be selected from the group consisting of, but not limited by cisplatin, caeboplatin, vinblastine, navelbine, imatinib and etoposide. In another embodiment, the pharmaceutical composition of the invention may be co-administered with other conventional anticancer treatments including, but not limited to anti-cancer antibodies (such as trastuzumab, alemtuzumab, bevacizumab, cetuximab or panitumumab) or anti-tumour vaccination protocols (via tumour antigen-presentation by dendritic cells or passive immunization via anti-tumour cell-specific biologicals or antibodies as described above).

In a further aspect, the present invention relates to method for identifying immunoaugmenting agents comprising: (a) contacting a cell, tissue or a non-human animal comprising a reporter construct for NR2F6-inhibition with a candidate molecule; (b) measuring the reporter signal; and (c) selecting a candidate molecule which alters the reporter signal.

Exemplary reporter signals, reporters and reporter constructs are described herein below. Interesting reporters, namely reporter gene products, which can be used in the screening and identifying methods of the invention like luciferase, (green/red) fluorescent protein and variants thereof, EGFP (enhanced green fluorescent protein), RFP (red fluorescent protein, like DsRed or DsRed2), CFP (cyan fluorescent protein), BFP (blue green fluorescent protein), YFP (yellow fluorescent protein), β-galactosidase or chloramphenicol acetyltransferase as well as methods for their detection are also described herein below in detail. The nucleic acid and amino acid sequence of an exemplary luciferase which can be advantageously used in accordance with the present invention (as also demonstrated in the appended examples) is depicted in SEQ ID NOs 32 and 33, respectively. Luciferase is a well known reporter; see, for example, Jeffrey (1987) Mol. Cell. Biol. 7(2), 725-737. A person skilled in the art can easily deduce further luciferase nucleic and amino acid sequences to be used in context of the present invention from corresponding databases and standard text books/review.

Further exemplary reporter constructs to be employed in context of the present invention, in particular the screening and identifying methods, comprise reporter(s) as defined herein and promoter(s) (and/or (a) enhancer region(s)) of(or recognized by) NR2F6 (or a functional fragment thereof) or of (a) component(s) of the NR2F6-dependent signaling pathway, wherein the (initiation/enhancement of the) expression of the reporter(s) is under control of the promoter and/or enhancer. Exemplary and non-limiting examples of such promoters are the human IL-17A promoter (depicted in SEQ ID NO: 29) or the RAR (retinoic acid receptor) regulated promoter (enhancer), whereas an exemplary enhancer sequence to be used in this context is the NF-AT/AP-1 enhancer sequence (which is formed by 3 tandem repeats of the NFAT/AP-1 consensus oligonucleotide from IL-2 distal site as shown in SEQ ID NO: 28). A skilled person may easily retrieve these and other well-known sequences from databases (like NCBI) and use these sequences in the generation of reporter constructs to be employed herein. For example, the IL-17 promoter sequence (SEQ ID NO: 29) can be retrieved under Accession number AY630567 from the corresponding database. An exemplary IL-17 promoter sequence to be used in context of the present invention is also described in Liu (2004), JBC, wherein the structure of a IL-17A promoter is disclosed. Therein the following characteristics of this promoter are described: 1.2 kb of 5-flanking sequence upstream of the transcriptional start point was cloned from genomic DNA. A number of predicted transcription factor binding sites were identified, including AP-1, NF-kappaB, and NF-AT. The TATA box and translation starting point are shown in this article.

It is shown in the appended examples that these reporter constructs comprising a reporter and a promoter (and/or enhancer) as defined above, are particularly useful in screening methods and assays, since the reporter signal associated with the reporter can easily be detected. Also fusion proteins (comprising a reporter and NR2F6 (or a functional fragment) or (a) component of the NR2F6-dependent signaling pathway) can be used as reporter constructs in this context. Such fusion proteins are described herein below in more detail. A change in the reporter signal is indicative for the capacity of a candidate molecule tested to act as antagonist/inhibitor of NR2F6. For example, an antagonist of NR2F6 will lead to a decrease of a reporter signal/activity of a reporter under control of the NR2F6 promoter region. Similarly, and as also demonstrated in the appended examples, an increase in the reporter signal/activity of a reporter under control of a promoter/enhancer region of (a) component(s) of the NR2F6-dependent signaling pathway is indicative for the capacity of the candidate molecule to act as antagonist/inhibitor of NR2F6 and, hence, to augment an immune response. Exemplary reporter constructs are provided and described in the appended example. In particular, a reporter construct comprising a luciferase gene and a promoter of (a) component(s) of the NR2F6-dependent signaling pathway, such as IL-17, may be employed. The generation of an exemplary reporter construct comprising the human IL-17 promoter and luciferase gene is described in the appended examples. A person skilled in the art is easily in the position to generate this and other reporter constructs using routine techniques. Inter alia, vectors such as the pRL-TK RENILLA Vector (commercially available from Promega Corporation, Madison, Wis., USA and shown in SEQ ID NO: 27) and other well known vectors may be employed in the generation of the reporter constructs.

The exemplary pRL-TK RENILLA Vector which may, inter alia, be employed in the generation of the herein described reporter construct is well known in the art (see, for example Matuszyk (2002), Biochem Biophys Res Commun, 294(5),1036-9. and described herein below in more detail. This vector contains 2 perfect match RARE (Retinoic Acid-Receptor Enhancer sites, TGACCT,) site, recognized by nuclear receptors such as NR2F6.

pRL-TK Vector sequence reference points are given herein below:

| | |
|---|---|
| Base pairs | 4045 |
| HSV TK promoter | 7-759 |
| Chimeric intron | 826-962 |
| T7 RNA polymerase Promoter (−17 to +2) | 1006-1024 |
| T7 RNA polymerase transcription initiation site | 1023 |
| Rluc reporter gene | 1034-1969 |
| SV40 late polyadenylation signal | 2011-2212 |
| Beta-lactamase (AmpR) | 2359-3219 |

It is preferred that promoter(s) and/or enhancer elements/regions of the following component(s) of the NR2F6-dependent signaling pathway are used in this context: NF-AT, AP-1, IL-2, IL-17 and IFN-gamma. However, further promoter(s)/enhancer(s) of other components of this signaling pathway may be used. Other components of this signaling pathway are shown in FIG. 25 and a skilled person is easily in the position to deduce promoter(s)/enhancer(s) of these components from databases. All explanation given herein below regarding the measurement of reporter signals/activity also apply here, mutatis mutandis.

As used herein, the term “immunoaugmenting agent” relates to compounds/molecules capable of augmenting the “immune response” or “immune reaction” as defined herein above. Accordingly, an immunoaugmenting agent is capable of enhancing/augmenting parameters which are indicative for an (augmented) magnitude of the immune response/reaction. Parameters indicative for the magnitude of the immune response/reaction may include, but are not limited to the presence/quantity of (specific) antibodies, presence/quantity of (specific) immune cells, the presence/quantity of (specific) cytokines and/or the presence/quantity of (specific) regulatory-, activation- and/or adhesion molecules.

As used herein, the term “reporter construct for NR2F6-inhibition” relates to any biotechnologically engineered construct allowing the detection of NR2F6 Accordingly, said reporter construct may allow the detection of NR2F6-inhibition by inducing a change in the signal strength of a detectable signal. Said detectable signal may be selected from the group consisting of, but not limited to a fluorescence resonance energy transfer (FRET) signal, a fluorescence polarization (FP) signal and a scintillation proximity (SP) signal as defined herein below. In a further embodiment, said detectable signal may be associated with a reporter gene product. Examples of reporter gene products include luciferase, (green/red) fluorescent protein and variants thereof, like EGFP (enhanced green fluorescent protein), RFP (red fluorescent protein, like DsRed or DsRed2), CFP (cyan fluorescent protein), BFP (blue green fluorescent protein), YFP (yellow fluorescent protein), β-galactosidase or chloramphenicol acetyltransferase, and the like. For example, GFP can be derived from Aequorea victoria (U.S. Pat. No. 5,491,084). A plasmid encoding the GFP of Aequorea victoria is available from the ATCC Accession No. 87451. Other mutated forms of this GFP including, but not limited to, pRSGFP, EGFP, RFP/DsRed, DSRed2, and EYFP, BFP, YFP, among others, are commercially available from, inter alia, Clontech Laboratories, Inc. (Palo Alto, Calif.). In one embodiment, said reporter construct for NR2F6-inhibition is selected from the group consisting of: a ligand-mediated reporter gene expression construct, a ligand displacement construct, a fluorescent cellular sensor fusion mutant construct, and a ligand-induced homo- and/or heterodimer construct.

Advantageous reporter constructs which can be used in the screening/identifying methods of the present invention are used and described in the appended examples. These reporter constructs may comprise reporters, namely reporter gene products, like luciferase, (green/red) fluorescent protein and variants thereof, EGFP (enhanced green fluorescent protein), RFP (red fluorescent protein, like DsRed or DsRed2), CFP (cyan fluorescent protein), BFP (blue green fluorescent protein), YFP (yellow fluorescent protein), β-galactosidase or chloramphenicol acetyltransferase. These reporters as well as methods for their detection are also described herein below in detail. Exemplary, non-limiting reporter constructs which are also used in the appended examples are constructs comprising a luciferase reporter under control of a (human) IL-17 promoter or under control of a NF-AT/AP-1 promoter and/or enhancer region.

In a preferred embodiment, the ligand-mediated reporter gene expression construct comprises an NR2F6 promoter reporter and consecutively expressed NR2F6, whereby a change in ligand binding to said NR2F6 promoter reporter leads to a change in reporter signal. Accordingly, test cells/tissues may be genetically engineered to contain non-endogenous DNA expressing NR2F6 or a functional fragment thereof and a DNA sequence coding a NR2F6 response element operatively linked to a reporter gene. The cultured cells/tissues are monitored for evidence of transcription of the reporter gene as a function of the concentration of test compound in the culture medium. The variation in transcription levels of the reporter gene as a function of the concentration of test compound indicates the ability of test compound to inhibit NR2F6 activity on target gene transcription.

Again, exemplary, non-limiting reporter constructs to be used in the present screening and identifying methods are constructs comprising a luciferase reporter under control of a (human) IL-17 promoter or a NF-AT/AP-1 promoter and/or enhancer region (it is envisaged that the latter fusion protein can be under control of a NF-AT/AP-1 promoter and/or enhancer region). A person skilled in the art is easily in the position to generate and use further reporter constructs based on the teaching given herein.

In another preferred embodiment, the ligand-mediated reporter gene expression construct comprises a NR2F6-dependent promoter reporter and consecutively expressed NR2F6, whereby a change in ligand binding to said NR2F6-dependent promoter reporter leads to a change in reporter signal. Accordingly, test cells/tissues may be genetically engineered to contain non-endogenous DNA expressing NR2F6 or a functional fragment thereof and a DNA sequence coding a NR2F6 response element operatively linked to a reporter gene. The cultured cells/tissues are monitored for evidence of transcription of the reporter gene as a function of the concentration of test compound in the culture medium. The variation in transcription levels of the reporter gene as a function of the concentration of test compound indicates the ability of test compound to inhibit NR2F6 activity on target gene transcription.

Also in this context, it is envisaged to use in the methods described herein cell(s), tissue(s) or non-human animal(s) which comprise the corresponding reporter construct(s). These cell(s), tissue(s) or non-human animal(s) are preferably transgenic. Such cell(s), tissue(s) or non-human animal(s) are particularly useful in methods for identifying NR2F6 antagonists/inhibitors and in corresponding screening methods.

In another preferred embodiment, the ligand displacement construct comprises ligand-binding domain of NR2F6 and a nuclear receptor-ligand or co-receptor protein, whereby displacement of said nuclear receptor-ligand or co-receptor protein from said ligand-binding domain of NR2F6 leads to a fluorescence polarization (FP) or scintillation proximity (SP) signal. In a preferred embodiment, said ligand-binding domain of NR2F6 represents a polypeptide encoded by the nucleic acid sequence SEQ ID NO:3 or having the amino acid sequence SEQ ID NO: 4 or a functional fragment thereof. Fluorescence polarization (FP) has been previously used to develop high-throughput screening (HTS) assays for nuclear receptor-ligand displacement employing purified ligand binding domain of nuclear receptors. Accordingly, recombinant NR2F6 protein of a functional fragment thereof (such as the ligand-binding domain) is kept in a buffer system with a fixed concentration of at least one established ligand of NR2F6, which is fluorescein-labelled.

The NR2F6 receptor binding assay is then based on the competition of compounds derived from a large library with the fluorescein-labelled ligand to bind NR2F6. An FP-based competitive binding assay can be used to screen diverse compounds with a broad range of binding affinities for NR2F6. Other examples of competition binding assays include SP assays where NR2F6 is bound to a scintillation proximity assay (SPA) bead in the presence of radiolabelled ligand. Compounds that directly interact with NR2F6 will displace the radiolabelled ligand leading to a detectable signal. Other marking and measuring techniques for NR2F6 interaction known in the art and/or disclosed herein may also be used.

In another preferred embodiment, the fluorescent cellular sensor fusion mutant construct comprises a reporter protein that is fused to a ligand-binding domain of NR2F6, whereby binding of a ligand to said ligand-binding domain of NR2F6 leads to a change in reporter signal. In another embodiment, said reporter signal is detectable in an interaction assay as described herein below.

Interaction assays employing read-out systems are well known in the art and comprise, inter alia, two hybrid screenings (as, described, inter alia, in EP-0 963 376, WO 98/25947, WO 00/02911), GST-pull-down columns, co-precipitation assays from cell extracts as described, inter alia, in Kasus-Jacobi (2000) Oncogene 19:2052-2059, “interaction-trap” systems (as described, inter alia, in U.S. Pat. No. 6,004,746) expression cloning (e.g. lamda gtII), phage display (as described, inter alia, in U.S. Pat. No. 5,541,109), in vitro binding assays and the like. Further interaction assay methods and corresponding read out systems are, inter alia, described in U.S. Pat. No. 5,525,490, WO 99/51741, WO 00/17221, WO 00/14271, WO 00/05410 or Yeast Four hybrid assays as described in Sandrok (2001) JBC 276:35328-35333.

Said interaction assays for NR2F6 also comprise assays for FRET-assays, TR-FRETs (in “A homogenius time resolved fluorescence method for drug discovery” in: High throughput screening: the discovery of bioactive substances. Kolb (1997) J. Devlin. NY, Marcel Dekker 345-360) or commercially available assays, like “Amplified Luminescent Proximity Homogenous Assay”, BioSignal Packard. Furthermore, the yeast-2-hybrid (Y2H) system may be employed to elucidate further particular and specific interaction, association partners of NR2F6. Said interaction/association partners are further screened for their antagonistic/inhibiting effects.

Similarly, interacting molecules (for example) (poly)peptides may be deduced by cell-based techniques well known in the art. These assays comprise, inter alia, the expression of reporter gene constructs or “knock-in” assays, as described, for, e.g., the identification of drugs/small compounds influencing the (gene) expression of NR2F6. Said “knock-in” assays may comprise “knock-in” of NR2F6 (or (a) fragment(s) thereof) in tissue culture cells, as well as in (transgenic) animals. Examples for successful “knock-ins” are known in the art (see, inter alia, Tanaka (1999) Neurobiol. 41:524-539 or Monroe (1999) Immunity 11:201-212). Furthermore, biochemical assays may be employed which comprise, but are not limited to, binding of the NR2F6 (or (a) fragment(s) thereof) to other molecules/(poly)peptides, peptides or binding of the NR2F6 (or (a) fragment(s) thereof) to itself (themselves) (dimerizations, oligomerizations, multimerizations) and assaying said interactions by, inter alia, scintillation proximity assay (SPA) or homogenous time-resolved fluorescence assay (HTRFA).

Said “testing of interaction” may also comprise the measurement of a complex formation. The measurement of a complex formation is well known in the art and comprises, inter alia, heterogeneous and homogeneous assays. Homogeneous assays comprise assays wherein the binding partners remain in solution and comprise assays, like agglutination assays. Heterogeneous assays comprise assays like, inter alia, immuno assays, for example, ELISAs, RIAs, IRMAs, FIAs, CLIAs or ECLs.

As discussed herein, the interaction of the antagonistic molecules of NR2F6 mRNA and NR2F6 protein or fragments thereof may also be tested by molecular biological methods, like two-, three- or four-hybrid-assays, RNA protection assays, Northern blots, Western blots, micro-, macro- and protein- or antibody arrays, dot blot assays, in situ hybridization and immunohistochemistry, quantitative PCR, coprecipitation, far western blotting, phage based expression cloning, surface plasmon resonance measurements, yeast one hybrid screening, DNAse 1, footprint analysis, mobility shift DNA-binding assays, gel filtration chromatography, affinity chromatography, immunoprecipitation, one- or two dimensional gel electrophoresis, aptamer technologies, as well as high throughput synthesis and screening methods.

In yet another preferred embodiment, the ligand-induced homo- and/or heterodimer construct comprises a NR2F6 or a part thereof capable of dimerization and a known dimerization partner of NR2F6, whereby dimerization of said NR2F6 or a part thereof capable of dimerization and said known dimerization partner of NR2F6 leads to a detectable interaction as defined herein above or an interaction signal In a preferred embodiment, said interaction signal is a fluorescence resonance energy transfer (FRET) signal.

Accordingly, determination of a ligand for NR2F6 may comprise contacting a component to be tested with an isolated nuclear receptor ligand binding domain which is associated with a first marking component and a heterodimeric partner for the nuclear receptor ligand binding domain associated with a second marking component and measuring the interaction between the marking components to determine whether the component to be tested modifies heterodimerization.

As already indicated herein above, various known markers may be used in the screening methods of the present invention. The first marking component may be a radioactive marker and the second marking agent may be a scintillation proximity analysis (SPA) bead. The interaction of the markers in this case is determined by scintillation proximity. Alternatively, the first marking component may be a first fluorescent dye emitting at an emitting wavelength which excites the second marking component which may be a second fluorescent dye. The interaction of the markers in this case is determined by homogenous time-resolved fluorimetry. The interaction of the marking components in either case is measured by comparing signal produced by a combination of the heterodimeric partner, the isolated nuclear receptor or a functional fragment thereof such as the ligand binding domain and the component to be tested with a signal produced by a combination of the heterodimeric partner, the isolated nuclear receptor or a functional fragment thereof such as the ligand binding domain in absence of the compound to be tested.

Another aspect of the present invention is a nuclear receptor-peptide-receptor protein assay for identifying ligands. This assay utilizes FRET as described herein above and thus can be tested for identifying putative ligands for NR2F6. The assay is based on the principle that ligands induce conformational changes in nuclear receptors that facilitate interactions with coactivator proteins required for transcriptional activation.

Since NR2F6 may also bind to DNA as a heterodimer with other nuclear receptors and/or coreceptor proteins (and functional fragments thereof), dimerization partners of NR2F6 may be used in the screening methods of the present invention. Said dimerization partners partners (which may bind to a given DNA promoter sequence) comprise, but are not limited to PPAR, RXR, RAR, VDR, T3R, NF-AT, AP-1 and Nur77. In another embodiment said dimerization partners (which may bind to a given DNA promoter sequence) may be selected from the group consisting of NF-AT, AP-1 and FOXP3 family members. It is preferable that said dimerization partners and the nuclear receptor ligand binding domains are recombinantly expressed proteins.

If a sample (collection of compounds) containing (a) compound(s) is identified in the art as a specific binding molecule capable of inhibiting the physiologic activity of NR2F6 (NR2F6 antagonist), then it is either possible to isolate the compound from the original sample identified as containing the compound in question or one can further subdivide the original sample, e.g., if it consists of a plurality of different compounds, so as to reduce the number of different substances per sample and repeat the method with the subdivisions of the original sample. It can then be determined whether said sample or compound displays the desired properties, i.e. the inhibition of NR2F6 function, by methods known in the art. Depending on the complexity of the samples, the steps described above can be performed several times, preferably until the sample identified according to the screening method only comprises a limited number of or only one substance(s). Preferably said sample comprises substances of similar chemical and/or physical properties, and most preferably said substances are identical.

The term “candidate molecule” as used herein refers to a molecule or substance or compound or composition or agent or any combination thereof to be tested by one or more screening method(s) of the invention as a putative antagonist or inhibitor of NR2F6 function, activity or expression. A test compound can be any chemical, such as an inorganic chemical, an organic chemical, a protein, a peptide, a carbohydrate, a lipid, or a combination thereof or any of the compounds, compositions or agents described herein. It is to be understood that the term “candidate molecule” when used in the context of the present invention is interchangeable with the terms “test compound”, “test molecule”, “test substance”, “potential candidate”, “candidate” or the terms mentioned herein above.

Also preferred potential candidate molecules or candidate mixtures of molecules to be used when contacting a cell expressing/comprising a reporter construct for NR2F6 inhibition as defined and described herein may be, inter alia, substances, compounds or compositions which are of chemical or biological origin, which are naturally occurring and/or which are synthetically, recombinantly and/or chemically produced. Thus, candidate molecules may be proteins, protein-fragments, peptides, amino acids and/or derivatives thereof or other compounds as defined herein, which bind to and/or interact with NR2F6, regulatory proteins/sequences of NR2F6 function or functional fragments thereof. Synthetic compound libraries are commercially available from Maybridge Chemical Co. (Trevillet, Cornwall, UK), Comgenex (Princeton, N.J.), Brandon Associates (Merrimack, N.H.), and Microsource (New Milford, Conn.). A rare chemical library is available from Aldrich (Milwaukee, Wis.). Alternatively, libraries of natural compounds in the form of bacterial, fungal, plant and animal extracts are available from e.g. Pan Laboratories (Bothell, Wash.) or MycoSearch (N.C.) are readily producible. Additionally, natural and synthetically produced libraries and compounds are readily modified through conventional chemical, physical, and biochemical means. Results obtained from deorphanisation programs based on phylogenetic analysis methods may aid to find the natural ligand for the NR2F6 orphan receptor and, e.g., will allow in silico profiling of potential ligands for NR2F6.

The generation of chemical libraries with potential ligands for NR2F6 is well known in the art. For example, combinatorial chemistry is used to generate a library of compounds to be screened in the assays described herein. A combinatorial chemical library is a collection of diverse chemical compounds generated by either chemical synthesis or biological synthesis by combining a number of chemical “building block” reagents. For example, a linear combinatorial chemical library such as a polypeptide library is formed by combining amino acids in every possible combination to yield peptides of a given length. Millions of chemical compounds can theoretically be synthesized through such combinatorial mixings of chemical building blocks. For example, one commentator observed that the systematic, combinatorial mixing of 100 interchangeable chemical building blocks results in the theoretical synthesis of 100 million tetrameric compounds or 10 billion pentameric compounds. (Gallop, Journal of Medicinal Chemistry, Vol. 37, No. 9, 1233-1250 (1994)). Other chemical libraries known to those in the art may also be used, including natural product libraries. Once generated, combinatorial libraries are screened for compounds that possess desirable biological properties. For example, compounds which may be useful as drugs or to develop drugs would likely have the ability to bind to the target protein identified, expressed and purified as described herein.

In the context of the present invention, libraries of compounds are screened to identify compounds that function as an antagonist or inhibitor of NR2F6. First, a library of small molecules is generated using methods of combinatorial library formation well known in the art. U.S. Pat. No. 5,463,564 and U.S. Pat. No. 5,574,656 are two such teachings. Then the library compounds are screened to identify those compounds that possess desired structural and functional properties. U.S. Pat. No. 5,684,711, discusses a method for screening libraries. To illustrate the screening process, the target cell or gene product and chemical compounds of the library are combined and permitted to interact with one another. A labelled substrate is added to the incubation. The label on the substrate is such that a detectable signal is emitted from metabolized substrate molecules. The emission of this signal permits one to measure the effect of the combinatorial library compounds on the enzymatic activity of target enzymes/activity of target protein by comparing it to the signal emitted in the absence of combinatorial library compounds. The characteristics of each library compound are encoded so that compounds demonstrating activity against the cell/enzyme/target protein can be analyzed and features common to the various compounds identified can be isolated and combined into future iterations of libraries. Once a library of compounds is screened, subsequent libraries are generated using those chemical building blocks that possess the features shown in the first round of screen to have activity against the target receptor. Using this method, subsequent iterations of candidate compounds will possess more and more of those structural and functional features required to inhibit the target receptor, until a group of antagonists/inhibitors with high specificity for the receptor can be found. These compounds can then be further tested for their safety and efficacy as an immunoaugmenting agent for use in animals, such as mammals. It will be readily appreciated that this particular screening methodology is exemplary only. Other methods are well known to those skilled in the art. For example, a wide variety of screening techniques are known for a large number of naturally-occurring targets when the biochemical function of the target protein is known. For example, some techniques involve the generation and use of small peptides to probe and analyze target proteins both biochemically and genetically in order to identify and develop drug leads. Such techniques include the methods described in WO 99/35494, WO 98/19162, WO 99/54728.

Furthermore, the present invention relates to a screening method of compounds suspected of being an antagonist of NR2F6 which comprises the measurement of the activity of NR2F6 or of an increase in the activity of components of the NR2F6-dependent signalling cascade.

Moreover, screening methods for antagonists/inhibitors of NR2F6 in cells, tissue and/or a non-human animal are provided. Also identification methods for antagonists of NR2F6 are provided. These methods are highly useful in identifying/screening (a) candidate molecule(s) suspected of being inhibitors of NR2F6 activity. Potent inhibitors identified/screened by these methods can be used in the medical intervention of a disease related to an insufficient immune response as defined herein, in particular cancer. In accordance with the present invention, a candidate molecule that may be suspected of being an antagonist of NR2F6 can, in principle, be obtained from any source as defined herein. The candidate molecule(s) may be (a) naturally occurring substance(s) or (a) substance(s) produced by a transgenic organism and optionally purified to a certain degree and/or further modified as described herein. Practically, the candidate molecule may be taken from a compound library as they are routinely applied for screening processes.

Accordingly, the present invention relates to a method for assessing the activity of a candidate molecule suspected of being an antagonist of NR2F6 comprising the steps of:

(a) contacting a cell, tissue or a non-human animal comprising NR2F6 with said candidate molecule;

(b) detecting a decrease in NR2F6 activity or an increase in the activity and/or expression of at least one component of the NR2F6-dependent intracellular signalling cascade; and

(c) selecting a candidate molecule that decreases NR2F6 activity or increases the activity and/or expression of at least one component of the NR2F6-dependent intracellular signalling cascade;

wherein a decrease of the NR2F6 activity or increase of the activity and/or expression of at least one component of the NR2F6-dependent intracellular signalling cascade is indicative for the capacity of the selected molecule to augment immune response.

It is to be understood that the detected activity of NR2F6 and/or at least one component of the NR2F6-dependent intracellular signalling cascade is compared to a standard or reference value of NR2F6 activity or of the at least one component of the NR2F6-dependent intracellular signalling cascade respectively. The standard/reference value may be detected in a cell, tissue, or non-human animal as defined herein, which has not been contacted with a potential NR2F6 inhibitor or prior to the above contacting step. The decrease in the activity of NR2F6 and/or increase in the activity of at least one component of the NR2F6-dependent intracellular signalling cascade upon contacting with (a) candidate molecule(s) may also be compared to the decrease in NR2F6 activity or a increase in the activity of (a) component(s) of the NR2F6-dependent intracellular signal cascade induced by (a) routinely used reference compound(s). A skilled person is easily in the position to determine/assess whether the activity and/or expression of NR2F6 or of at least one component of the NR2F6-dependent intracellular signalling cascade is (preferably statistically significant) increased.

In accordance with this invention, in particular the screening or identifying methods described herein, a cell, tissue or non-human animal to be contacted with a candidate molecule comprises NR2F6. For example said cell, tissue or non-human animal may express a NR2F6 gene, in particular also (an) additional (copy) copies of a NR2F6 gene, (a) NR2F6 mutated gene(s), a recombinant NR2F6 gene construct and the like. As explained herein below, the capability of a candidate molecule to inhibit/antagonize NR2F6 may, accordingly, be detected by measuring the expression level of such gene products of NR2F6 or of corresponding gene constructs (e.g. mRNA or protein), wherein a low expression level (compared to a standard or reference value) is indicative for the capability of the candidate molecule to act as inhibitor/antagonist.

The term “comprising NR2F6” may, for example, relate to a reporter construct which comprises NR2F6 (or a functional fragment thereof) and a “reporter”. Exemplary reporters are described herein above in context of “reporter constructs for NR2F6-inhibition”. Interesting reporters, namely reporter gene products, which can be used in the screening methods of the invention like luciferase, (green/red) fluorescent protein and variants thereof, EGFP (enhanced green fluorescent protein), RFP (red fluorescent protein, like DsRed or DsRed2), CFP (cyan fluorescent protein), BFP (blue green fluorescent protein), YFP (yellow fluorescent protein), β-galactosidase or chloramphenicol acetyltransferase as well as methods for their detection are described herein above in detail.

The skilled person is readily in the position to generate and use also other reporters/reporter constructs which can be employed in accordance with the present invention.

The use of fusion proteins containing a NR2F6 protein (or a functional fragment thereof) and a reporter gene product is particularly envisaged in the methods of the present invention.

Antagonists of NR2F6 may interfere with the transcription of NR2F6 or with the transcription of a reporter construct, in particular NR2F6 fusion proteins. For example, the antagonists may bind to the promoter region of NR2F6 or of the fusion protein, thus preventing initiation of transcription or stopping the already initiated transcription process. The antagonists may also bind to/interfere with components of the transcription machinery, thereby effectively inhibiting initiation of transcription or continuation of transcription. Such an interference with the transcription of NR2F6, NR2F6 constructs or NR2F6 fusion proteins by a candidate molecule will be reflected in a decreased transcription activity and hence, a reduced transcript level (e.g. unspliced/partially spliced/spliced mRNA). It is also envisaged herein that a reporter construct to be used herein comprises the promoter of NR2F6 linked to a reporter as described herein. Thus, activity of NR2F6 may be reflected in an activation of its promoter and, hence, in turn reflected in the change/decrease of the reporter signal associated with the reporter.

Due to the reduced transcript level also the level of the translated gene product (i.e. the protein level) will be decreased. The level of the above described fusion proteins preferably correlates with the signal strength of a detectable signal associated with the reporter gene product. These signals have been disclosed and defined herein above in detail. Exemplary NR2F6 fusion proteins are proteins comprising NR2F6 (or a functional fragment thereof) and a reporter as described above (e.g. luciferase, (green/red) fluorescent protein and variants thereof, EGFP (enhanced green fluorescent protein), and the like).

Accordingly, a decrease in NR2F6 (promoter) activity (which may, for example, be reflected in a decrease in the (NR2F6 promoter) reporter signal) upon contacting the cell/tissue/non-human animal with a candidate molecule will indicate that the candidate molecule is indeed an NR2F6 inhibitor/antagonist and, thus, capable of augmenting the immune response. The candidate molecules which decrease NR2F6 (promoter) activity as defined herein above are selected out of the candidate molecules tested, wherein those molecules are preferably selected which strongly decrease NR2F6 (promoter) activity (reflected, for example, in a pronounced decrease in the reporter signal). It is assumed that the NR2F6 antagonizing/inhibiting activity of a candidate molecule is the stronger the more the reporter signal is decreased.

It is elucidated herein above that NR2F6 decreases the activity and/or expression of components of the NR2F6-dependent signaling pathway, i.e. in “downstream” evaluation. Accordingly, an antagonist/inhibitor of NR2F6 will lead to an increased activity and/or expression of said components. In the screening methods of the present invention, candidate molecules can, therefore, be selected as NR2F6 antagonists/inhibitors, if the activity and/or expression of said components is increased upon contacting the cell/tissue/non-human animal with the candidate molecule. For example, the activity of NF-AT and/or AP-1 is increased, which is, in turn reflected in increased expression levels of subsequent components of the NR2F6-dependent signaling pathway, e.g. IL-2, IL-17 and/or IFN-gamma. The activity of (a) component of the NR2F6-dependent signaling pathway can be reflected in its transcriptional activity (for example, NF-AT/AP-1 regulate transcription of further downstream components) and also in their DNA-binding capacity. For example, NF-AT/AP-1 bind in their active form to corresponding DNA enhancer sequence. The measurement of this DNA-binding (e.g. detecting/measuring the amount of the component of the NR2F6-dependent signaling pathway, such as NF-AT/AP-1) can be performed by methods known in the art e.g. EMSA and the like, and is also demonstrated in the appended examples. Binding of components of the NR2F6-dependent signalling pathway (e.g. NFAT/AP-1) to the corresponding DNA enhancer sequence is increased in the presence of an NR2F6 antagonist (compared to a reference/standard value).

However, also reporter constructs/reporters as described herein above in context of NR2F6 may be employed in the context of “downstream” read-outs. For example, the cell/tissue/non-human animal may contain (a) reporter construct which comprises a component of the NR2F6-dependent signalling pathway and a reporter. Also in this context the use of fusion proteins is advantageous as shown in the appended examples. An exemplary NF-AT/AP-1 promoter/enhancer region is provided and used in appended Example. Therein it is shown that the activity of luciferase is decreased upon contacting T-cells with NR2F6. Again a luciferase construct is only an exemplary construct. The person skilled in the art can readily make and employ other corresponding reporter constructs. It is envisaged that this exemplary fusion protein can be used to assess the activity of a candidate molecule suspected of being an antagonist of NR2F6. For example, the candidate molecule will lead to an increase in reporter activity/reporter signal (compared to a standard/reference value) if said molecule has indeed NR2F6 antagonizing/inhibiting activity.

It is envisaged in the context of the present invention (in particular the screening/identifying methods disclosed herein) that also cellular extracts can be contacted (e.g. cellular extracts comprising NR2F6 as described and defined herein). For example, these cellular extracts may be obtained from the (transgenic/genetically engineered) cell(s), tissue(s) and/or non-human animal(s) to be used herein, in particular to be contacted with the candidate molecule. The use of such cellular extracts is particular advantageous since it allows the assessment of the activity of a candidate molecule in vitro. The assessing/screening methods taking advantage of such (cellular) extracts can, for example, be used in prescreening candidate molecules, wherein the molecules selected in such a prescreen are then subject to subsequent screens, for example in the cell-based methods disclosed herein, in particular in methods wherein a (transgenic) cell(s), tissue(s) and/or non-human animal(s) are contacted with a candidate molecule. In this context, it is accordingly preferred that the candidate molecule has been selected in the in vitro pre-screening method, described herein above and below.

Accordingly, the present invention relates in one embodiment to an (in vitro) method for assessing the activity of a candidate molecule suspected of being an antagonist of NR2F6 comprising the steps of:
(a) contacting a cellular extract comprising NR2F6 with said candidate molecule;
(b) detecting a decrease in NR2F6 activity or an increase in the activity and/or expression of at least one component of the NR2F6-dependent intracellular signalling cascade; and
(c) selecting a candidate molecule that decreases NR2F6 activity or increases the activity and/or expression of at least one component of the NR2F6-dependent intracellular signalling cascade;
   wherein a decrease of the NR2F6 activity or increase of the activity and/or expression of at least one component of the NR2F6-dependent intracellular signalling cascade is indicative for the capacity of the selected molecule to augment immune response.

All the explanations given herein in context of the identifying/assessing/screening methods apply also here, mutatis mutandis. A person skilled in the art is easily in the position to prepare such (cellular) extracts by routine techniques and to employ these extracts in accordance with the present invention (in particular in the screening/identifying methods provided herein).

In particular in the context of screening methods employing cellular extracts it is envisaged that the activity of NR2F6 and/or activity of (a) component(s) of the NR2F6-dependent signalling pathway can be detected by (direct) DNA-binding assays (which is also illustrated in the appended examples). These DNA-binding assays are also described elsewhere herein. These assays comprise, in particular, determining whether the amount of binding of a component of the NR2F6-dependent signalling pathway (e.g. NF-AT and/or AP-1) to their cognate DNA enhancer sequence in vitro is increased by a candidate molecule suspected of being an antagonist of NR2F6 compared to a standard/reference value. As also shown in the appended examples, the DNA-binding can be measured/detected by EMSA. In particular in High-through-put screening the detection of a reporter signal associated with DNA binding is envisaged, using e.g. scintillation proximity, ELISA, fluorescence polarization assay, FRET assay, and the like. NF-AT/AP-1 binding may, for example, lead to a fluorescence polarization (FP) or scintillation proximity (SP) signal.

The sample (e.g. cell(s), tissue(s), non-human-animal) is contacted with (a) candidate molecule(s) to be tested and it is measured whether said candidate molecule(s) lead(s) to a decrease in the activity of NR2F6 or leads to a change (in particular an increase) in the activity of components of the NR2F6-dependent intracellular signal cascade. Such a change/increase is indicative for the capacity of the candidate molecule to augment an immune response. In other words, the activity of the candidate molecule(s) as inhibitors/antagonists of NR2F6 is assessed based on their capacity to decrease the activity of NR2F6 or an increase in the activity of components of the NR2F6-dependent intracellular signal cascade. In particular the use of (a) (transgenic) cell(s), tissue(s), or non-human animal(s) overexpressing NR2F6 is envisaged, since these may allow a more sensitive/easier detection of a decrease of NR2F6 activity or increase in the activity and/or expression of at least one component of the NR2F6-dependent intracellular signalling cascade.

It is to be understood that in a high throughput screening routinely, many (often thousands of candidate molecules) are screened simultaneously. Accordingly, in a (first) screen candidate molecules are selected, which decrease NR2F6 activity or change/increase the activity of component(s) of the NR2F6-dependent signalling cascade.

Step (a) of the screening methods of the present invention, i.e. the “contacting step” may also be accomplished by adding a (biological) sample or composition containing said candidate molecule or a plurality of candidate molecules (i.e. various different candidate molecules) to the sample to be analyzed (e.g. (a) cell(s)/tissue(s)/non-human animal comprising NR2F6 or a functional fragment thereof).

Exemplary, non-limiting reporter constructs for “downstream” readout to be used in the present screening and identifying methods are constructs comprising a luciferase reporter under control of a (human) IL-17 promoter or a NF-AT/AP-1 promoter and/or enhancer region). The generation and use of such constructs is also described and shown in the appended examples. Again, these constructs are only of exemplary nature and a person skilled in the art is easily in the position to generate and use further reporter constructs based on the teaching given herein.

The term “contacting” may also refer to the addition of a candidate molecule to a sample to be analyzed (e.g., a cell, tissue, non-human animal comprising NR2F6) in a way that the candidate molecule may become effective to the cell at the cell surface or upon cellular uptake and thereby exert its inhibitory function on NR2F6-dependent T cell responses.

Generally, the candidate molecule(s) or a composition comprising/containing the candidate molecule(s) may for example be added to a (transfected) cell, tissue or non-human animal comprising NR2F6. As defined and disclosed herein, the term “comprising NR2F6” refers not only to the NR2F6 gene(s) or proteins known in the art and described herein, but also to reporter constructs comprising a reporter and NR2F6. Exemplary reporters (preferably associated with the reporter signals disclosed herein) are luciferase and fluorescent proteins, like GFP, RFP and the like. Also reporter constructs comprising a promoter and/or enhancer region of NR2F6 or of (a) component(s) of the NR2F6-dependent signalling pathway and a reporter as defined herein can be used in the screening/identifying methods. Accordingly, the cell(s), tissue(s) and/or non-human animals used in the context of the present invention, in particular in context of the screening/identifying methods can be stably or transiently transfected with the reporter constructs disclosed herein. Again, exemplary, non-limiting constructs to be used may comprise a luciferase reporter under control of a (human) IL-17 promoter or a NF-AT/AP-1 promoter and/or enhancer region.

In particular the identification/assessment of candidate molecules which are capable of inhibiting/antagonizing NR2F6 may be, inter alia, performed by transfecting an appropriate host with a nucleic acid molecule encoding NR2F6 (or a functional fragment thereof) and/or a nucleic acid molecule encoding a component of the NR2F6-dependent signaling pathway and constacting said host with (a) candidate molecule(s). Exemplary components of the signaling pathway are NF-AT, AP-1, IL-2, IL-17 and IFN-gamma. Further components can be deduced from FIG. 25. The host (cell, tissue, non-human animal) can also be transfected with the above described reporter constructs, e.g. luciferase reporter constructs, such as, but not limited to, reporter constructs comprising a luciferase reporter under control of a (human) IL-17 promoter or a NF-AT/AP-1 promoter and/or enhancer region. The host comprises, but is not limited to any cell of the immune system, but may also comprise CHO-cell, HEK 293, HeLa, Cos 7, PC12 or NIH3T3 cell, frog oocytes or primary cells like primary cardiomyocytes, fibroblasts, muscle, endothelial or embryonic stem cells. Alternatively, it is also possible to use cell lines stably transfected with a nucleic acid molecule encoding NR2F6 or a functional fragment thereof. In a preferred embodiment of the present invention, the cell or the cell line used for stable transfection or transiently transfected with a nucleic acid molecule encoding NR2F6 or a functional fragment thereof is a Jurkat T cell or a cell line comprising or derived from Jurkat T cells. The explanations given herein above in respect of “cells” also apply to tissues/non-human animals comprising or derived from these cells. A sample to be analyzed may also be a biological, medical or pathological sample, for example fluids that comprise cells, tissues or cell cultures. Such a fluid may be a body fluid or also excrements and may also be a culture sample. The body fluids may comprise but are not limited to blood, serum, plasma, urine, saliva, synovial fluid, spinal fluid, cerebrospinal fluid, tears, stool and the like.

The (biological) sample or composition, comprising a plurality of candidate molecules are usually subject to a first screen. The samples/compositions tested positive in the first screen are often subject to subsequent screens in order to verify the previous findings and to select the most potent inhibitors/antagonists of NR2F6. Upon multiple screening and selection rounds those candidate molecules will be selected which show a pronounced capacity to inhibit/antagonize NR2F6 as defined and disclosed herein. For example, batches (i.e. compositions/samples) containing many candidate molecules will be rescreened and batches with no or insufficient inhibitory activity of candidate molecules be discarded without re-testing.

For example, if a (biological) sample or composition with many different candidate molecules is tested and one (biological) sample or composition is tested positive, then it is either possible in a second screening to screen, preferably after purification, the individual molecule(s) of the (biological) sample or composition. It may also be possible to screen subgroups of the (biological) sample or composition of the first screen in (a) subsequent screen(s). The screening of compositions with subgroups of those candidate molecules tested in previous screening rounds will thus narrow in on (an) potential potent NR2F6 inhibitor(s). This may facilitate and accelerate the screening process in particular when a large number of molecules is screened. Accordingly, the cycle number of screening rounds is reduced compared to testing each and every individual candidate molecule in (a) first (and subsequent) screen(s) (which is, of course, also possible). Thus, depending on the complexity or the number of the candidate molecules, the steps of the screening method described herein can be performed several times until the (biological) sample or composition to be screened comprises a limited number, preferably only one substance which is indicative for the capacity of screened molecule to augment an insufficient immune response.

The term “decrease in NR2F6 activity” in step (b) of the screening method means that the “activity of NR2F6” is reduced upon contacting the cell, tissue, or non-human animal comprising NR2F6 with the candidate molecule, preferably in comparison to a (control) standard or reference value, as defined herein. Similarly, the term “increase in the activity of at least one component of the NR2F6-dependent signalling pathway” (i.e. of one or more of said components) in step (b) of the screening method means that the “activity” of at least one component of the NR2F6-dependent signalling pathway is increased upon contacting the cell, tissue, or non-human animal comprising NR2F6 with the candidate molecule, preferably in comparison to a (control) standard or reference value as defined herein.

As defined and disclosed herein, the term “comprising NR2F6” refers not only to the NR2F6 gene(s) or proteins known in the art and described herein, but also to reporter constructs comprising a reporter and NR2F6. Exemplary reporters (preferably associated with the reporter signals disclosed herein) are luciferase and fluorescent proteins, like GFP, RFP and the like. Also reporter constructs comprising a promoter and/or enhancer region of NR2F6 or of (a) component(s) of the NR2F6-dependent signalling pathway and a reporter as defined herein can be used in the screening/identifying methods. Accordingly, the cell(s), tissue(s) and/or non-human animals used in the context of the present invention, in particular in context of the screening/identifying methods can comprise the reporter constructs disclosed herein. Again, exemplary, non-limiting constructs to be used may comprise a luciferase reporter under control of a (human) IL-17 promoter or a NF-AT/AP-1 promoter and/or enhancer region.

Particularly preferred are optical measurement techniques that allow a resolution of fluorescence on the level of single cells or single cells of a tissue, preferably at the subcellular level. They may involve fluorescence, for example confocal microcopy, digital image recording, preferably a CCD camera and suitable picture analysis software. Preferably, step (b) is carried out after the measurement of a standard response by performing a control experiment. For example, the activity of NR2F6 or the activity of (a) component(s) of the NR2F6-dependent signalling cascade is measured in a cell, tissue or a non-human animal comprising NR2F6 without contacting a candidate molecule in a first screen. In a second screen, after contacting the candidate molecule, the activity of NR2F6 or the activity of (a) component(s) of the NR2F6-dependent signalling cascade is measured. A difference in the activities will indicate whether the tested candidate molecule is indeed an antagonist of NR2F6 and capable of augmenting the immune response.

The activity of NR2F6 or of (a) component(s) of the NR2F6-dependent signalling pathway can be quantified by measuring, for example, the level of gene products (e.g. mRNA and/or protein of NR2F6 and said component, respectively) by any of the herein described methods, activities, the interleukin concentration or other cellular functions, like inter alia, the involvement in signalling pathways or changes in intracellular localization. Preferably, the stimulation/activation of signaling pathways, in particular the activation of NF-AT, AP-1 and expression of IL-2, IL-17 and/or IFN gamma is measured. As mentioned herein above the candidate compound to be tested may lead to a modified (DNA-binding and transcriptional activity and/or expression) of NF-AT, AP-1, and subsequently expression of IL-2, IL-17 and/or IFN gamma, wherein for example an increased activity and/or expression of NF-AT, AP-1, IL-2, IL-17 and/or IFN gamma or a decrease in the NR2F6 activity are indicative for the capacity to antagonize NR2F6 and thus to augment an immune response. Preferably, the increased activity and/or expression of NF-AT, AP-1, IL-2, IL-17 and/or IFN gamma and/or a decrease in the NR2F6 activity is measured in comparison to the (control) standard value.

As mentioned, a “decreased NR2F6 activity” and, accordingly, a decreased concentration/amount of NR2F6 proteins in a sample may be reflected in a decreased expression of the corresponding gene(s) encoding the NR2F6 protein(s). Therefore, a quantitative assessment of the gene product (e.g. protein or spliced, unspliced or partially spliced mRNA) can be performed in order to evaluate decreased expression of the corresponding gene(s) encoding the NR2F6 protein(s). Also here, a person skilled in the art is aware of standard methods to be used in this context or may deduce these methods from standard textbooks (e.g. Sambrook, 2001, loc. cit.). For example, quantitative data on the respective concentration/amounts of mRNA from NR2F6 can be obtained by Northern Blot, Real Time PCR and the like. Preferably, the concentration/amount of the gene product (e.g. the herein above described NR2F6 mRNA or NR2F6 protein) may be decreased by at least about 10%, 20%, 30%, 40%, preferably by at least 50%, 60%, 70%, 80%, 90%, or 100% compared to a control sample. It is preferred herein that NR2F6 proteins are (biologically) active or functional. Methods for determining the activity of NR2F6 are described herein above and shown in the appended example. Since the NR2F6 proteins are preferably (biologically) active/functional (wherein it is preferred that at least 70%, 75%, preferably at least 80%, 85%, 90%, 95%, 96, %, 97%, 98% and most preferably, at least 99% of NR2F6 proteins of a sample a (biologically) active/functional), an decreased concentration/amount of NR2F6 proteins in a sample reflects a decreased (biological) activity of the NR2F6 protein, and consequently an increased (biological) activity of the components of the NR2F6-dependent signalling cascade and vice versa. The explanations given in respect of “activity of NR2F6” also apply, mutatis mutandis, to the “activity of (a) component(s) of the NR2F6-dependent signalling cascade”.

As mentioned, a person skilled in the art is aware of standard methods to be used for determining or quantitating expression of a nucleic acid molecule encoding, for example, the NR2F6 (or fragments thereof) or components of the NR2F6-dependent signalling cascade (or fragments thereof) as defined herein. For example, the expression can be determined on the protein level by taking advantage of immunoagglutination, immunoprecipitation (e.g. immunodiffusion, immunclectrophoresis, immune fixation), western blotting techniques (e.g. (in situ) immune histochemistry, (in situ) immune cytochemistry, affinitychromatography, enzyme immunoassays), and the like. Amounts of purified polypeptide in solution can be determined by physical methods, e.g. photometry. Methods of quantifying a particular polypeptide in a mixture rely on specific binding, e.g. of antibodies. Specific detection and quantitation methods exploiting the specificity of antibodies comprise for example immunohistochemistry (in situ). For example, concentration/amount of NR2F6 proteins in a cell, tissue or a non-human animal can be determined by enzyme linked-immunosorbent assay (ELISA). Alternatively, Western Blot analysis or immunohistochemical staining can be performed. Western blotting combines separation of a mixture of proteins by electrophoresis and specific detection with antibodies. Electrophoresis may be multi-dimensional such as 2D electrophoresis. Usually, polypeptides are separated in 2D electrophoresis by their apparent molecular weight along one dimension and by their isoelectric point along the other direction.

Expression can also be determined on the nucleic acid level (e.g. if the gene product/product of the coding nucleic acid sequence is an unspliced/partially spliced/spliced mRNA) by taking advantage of Northern blotting techniques or PCR techniques, like in-situ PCR or Real time PCR. Quantitative determination of mRNA can be performed by taking advantage of northern blotting techniques, hybridization on microarrays or DNA chips equipped with one or more probes or probe sets specific for mRNA transcripts or PCR techniques referred to above, like, for example, quantitative PCR techniques, such as Real time PCR.

These and other suitable methods for detection and/or determination of the concentration/amount of (specific) mRNA or protein(s)/polypeptide(s) are well known in the art and are, for example, described in Sambrook (2001), loc. cit.).

A skilled person is capable of determining the amount of mRNA or polypeptides/proteins, in particular the gene products described herein above, by taking advantage of a correlation, preferably a linear correlation, between the intensity of a detection signal and the amount of, for example, the mRNA or polypeptides/proteins to be determined.

Accordingly, the activity of NR2F6 (or a functional fragment thereof) or the activity of (a) component(s) of the NR2F6-dependent signalling cascade may be quantified based on the mRNA or protein level of NR2F6 or a functional fragment thereof and vice versa.

The term “signalling cascade” refers to any process by which a cell converts one kind of signal into another. Most processes of a signal cascade are transduced (carried out) by enzymes and activated by, for example second messengers, resulting in a signal transduction pathway. In particular, the number of proteins and other molecules participating in the events involving signal transduction increases as the process emanates from the initial stimulus, resulting in a “signalling cascade”. The NR2F6-dependent signalling cascade has also been described herein above in detail. Preferably, the components of said NR2F6-dependent signalling cascade are selected from the group consisting of NF-AT, AP-1, IL-2, IL-17 and IFN gamma. Further “downstream” components of the NRF2-dependent signaling cascade can be deduced from FIG. 25. Also these components (and reporter constructs disclosed herein comprising these components or promoter/enhancer regions of these components can be employed in accordance with the present invention, in particular in context of the screening/identifying methods).

In particular, reporter constructs comprising a promoter and/or enhancer region of NR2F6 or of (a) component(s) of the NR2F6-dependent signalling pathway and a reporter as defined herein can be used in the screening/identifying methods. Again, exemplary reporters (preferably associated with the reporter signals disclosed herein) are luciferase and fluorescent proteins, like GFP, RFP and the like. Exemplary, non-limiting constructs to be used may comprise a luciferase reporter under control of a (human) IL-17 promoter or a NF-AT/AP-1 promoter and/or enhancer region. The use of such constructs in screening methods is also demonstrated in the appended examples. Exemplary reporters (preferably associated with the reporter signals disclosed herein) are luciferase and fluorescent proteins, like GFP, RFP and the like. These and other reporters/reporter constructs/reporter signals are also described herein above and below.

The difference, as disclosed herein is statistically significant and a candidate molecule(s) is (are) selected, if the NR2F6 activity (or of a corresponding reporter signal) is strongly decreased, preferably is very low or non-dectable. For example, the NR2F6 activity (or of a corresponding reporter signal) may be decreased by at least 50%, 60%, 70%, 80%, more preferably by at least 90% compared to the (control) standard value. Analogously, a candidate molecule(s) is (are) selected, if the activity of any of the component(s) of the NR2F6-dependent signalling cascade strongly increased. For example the activity of any of the component(s) of the NR2F6-dependent signaling cascade may be increased by at least 50%, 60%, 70%, 80% or more preferably 90%. It is preferred that the activity of NF-AT, AP-1, and expression of IL-2, IL-17 and/or IFN gamma comprises at least about 1 fold (expression/amount), about 2 fold, about 3 fold, about 4 fold, about 5 fold, about 6 fold, about 7 fold, about 8 fold, about 9 fold, about 10 fold, about 20 fold, about 30 fold, about 40 fold, or about 50 fold compared to the (control) standard value. In a cell based method the cells can be transfected with one or more constructs encoding NR2F6 or a functional fragment thereof as described above and optionally a reporter under the transcriptional control of NR2F6 or a functional fragment thereof as described above. The successful use of reporter constructs is also demonstrated in the appended examples.

Preferably, the selected compound has a high NR2F6 inhibiting/antagonizing activity. This can be reflected in the capacity of the NR2F6 antagonist/inhibitor to potently decrease the activity of NR2F6. However, the NR2F6 antagonist/inhibitor may also interfere with the NR2F6-dependent signalling cascade, eventually leading to the activation of expression of transcription factors NF-AT and AP-1 which in turn induce the (expression of) key cytokine genes such as IL-2, IL-17 and IFN gamma.

The above detected difference between the activity of NR2F6 or the activity of a functional fragment of NR2F6 in a cell, tissue or a non-human animal contacted with said candidate molecule and the activity in the (control) standard value (measured e.g. in the absence of said candidate molecule) may be reflected by the presence, the absence, the increase or the decrease of a specific signal in the readout system, as in the herein described fluorescence based system.

Genetic readout systems are also envisaged. Analogously, the activity of NR2F6 or of a functional fragment thereof may be quantified by any molecular biological method as described herein. A skilled person is also aware of standard methods to be used in determining the amount/concentration of NR2F6 expression products (in particular the protein and the nucleic acid level of NR2F6) or the expression products of component(s) of the NR2F6-dependent signalling cascade in a sample or may deduce corresponding methods from standard textbooks (e.g. Sambrook, 2001).

In samples obtained from (a) cell(s), tissue or a cell culture(s) transfected with appropriate constructs or obtained from transgenic animals or cell cultures derived from a non-human animal(s), the concentration/amount of NR2F6 protein can be determined by bioassays, if, for example, a NR2F6-inducible promoter is fused to a reporter gene. Apparently, decreased expression of the reporter gene/activity of the reporter gene product will reflect a decreased NR2F6 activity, in particular a decreased concentration/amount of NR2F6 protein. An exemplary bioassay based on Jurkat T cells transiently transfected with the reporter gene luciferase under the control of the NR2F6 sensitive/inducible NF-AT:AP-1 promotor is also described in the appended example. As demonstrated in the example, an increase of NR2F6 leads to a decrease in the luciferase activity; see FIG. 15C. Alternatively, the effect of the NR2F6 protein on the expression of (a) reporter gene(s) may be evaluated by determining the amount/concentration of the gene product of the reporter gene(s) (e.g. protein or spliced, unspliced or partially spliced mRNA). Further methods to be used in the assessment of mRNA expression of a reporter gene are within the scope of a skilled person and also described herein below.

Also in this context, reporter constructs comprising a promoter and/or enhancer region of NR2F6 or of (a) component(s) of the NR2F6-dependent signalling pathway and a reporter as defined herein can be used in the screening/identifying methods. Exemplary reporters (preferably associated with the reporter signals disclosed herein) are luciferase and fluorescent proteins, like GFP, RFP and the like. Exemplary, non-limiting constructs to be used may comprise a luciferase reporter under control of a (human) IL-17 promoter or a NF-AT/AP-1 promoter and/or enhancer region. The use of such constructs in screening methods is also demonstrated in the appended examples. Exemplary reporters (preferably associated with the reporter signals disclosed herein) are luciferase and fluorescent proteins, like GFP, RFP and the like. These and other reporters/reporter constructs/reporter signals are also described herein above and below.

In sum, the present invention provides for the first time methods for identifying, and characterizing (a) candidate molecule(s) or (a) compound(s) which are capable of inhibiting/antagonizing NR2F6 whereby said inhibition may lead to an activation, a partial activation of the biological and/or pharmacological function of said NR2F6-dependent signalling cascade. Therefore the present invention provides for screening as well as identification methods for antagonists of said NR2F6-dependent signalling cascade. As also disclosed herein above, the term “antagonist” relates to molecules or compounds that bind to NR2F6 or a functional fragment thereof, thereby inhibitings and/or reducing NR2F6 activity (and leading, subsequently, to the activation of (a) component(s) of the NR2F6-dependent signalling cascade, wherein these NR2F6 antagonists are capable of augmenting an immune response.

Preferably, candidate agents to be tested encompass numerous chemical classes, though typically they are organic compounds, preferably small (organic) molecules as defined herein above.

Candidate agents may also comprise functional groups necessary for structural interaction with proteins, particularly hydrogen bonding, and typically include at least an amine, carbonyl, hydroxyl or carboxyl group, preferably at least two of the functional chemical groups. The candidate agents often comprise carbocyclic or heterocyclic structures and/or aromatic or polyaromatic structures substituted with one or more of the above functional groups.

Exemplary classes of candidate agents may include heterocycles, peptides, saccharides, steroids, and the like. The compounds may be modified to enhance efficacy, stability, pharmaceutical compatibility, and the like. Structural identification of an agent may be used to identify, generate, or screen additional agents. For example, where peptide agents are identified, they may be modified in a variety of ways to enhance their stability, such as using an unnatural amino acid, such as a D-amino acid, particularly D-alanine, by functionalizing the amino or carboxylic terminus, e.g. for the amino group, acylation or alkylation, and for the carboxyl group, esterification or amidification, or the like. Other methods of stabilization may include encapsulation, for example, in liposomes, etc.

As mentioned above, candidate agents are also found among other biomolecules including amino acids, fatty acids, purines, pyrimidines, nucleic acids and derivatives, structural analogs or combinations thereof. Candidate agents are obtained from a wide variety of sources including libraries of synthetic or natural compounds. For example, numerous means are available for random and directed synthesis of a wide variety of organic compounds and biomolecules, including expression of randomized oligonucleotides and oligopeptides. Alternatively, libraries of natural compounds in the form of bacterial, fungal, plant and animal extracts are available or readily produced. Additionally, natural or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, amidification, etc. to produce structural analogs.

The reporter constructs for detecting NR2F6 inhibition as described herein above may be comprised in a cell, tissue or a non-human animal. In a preferred embodiment, said reporter construct for detecting NR2F6 inhibition is comprised in Jurkat T-cells. Methods for transfecting cells or tissues are known in the art. Accordingly, calcium phosphate treatment or electroporation may be used for transfecting cells or tissues to express said reporter constructs (see Sambrook (1989), loc. cit.). Furthermore, nucleic acid molecules expressing said reporter constructs can be reconstituted into liposomes for delivery to target cells. As a further alternative, cells may be transduced to express specific reporter construct using genetically engineered viral vectors.

In another preferred embodiment, the non-human animal comprising said reporter construct for detecting NR2F6 inhibition is a transgenic non-human animal. The non-human organism to be used in the described screening assays is preferably selected from the group consisting of C. elegans, yeast, drosophila, zebrafish, guinea pig, rat and mouse. The generation of such a transgenic animal is within the skill of a skilled artisan. Corresponding techniques are, inter alia, described in “Current Protocols in Neuroscience” (2001), John Wiley & Sons, Chapter 3.16. Accordingly, the invention also relates to a method for the generation of a non-human transgenic animal comprising the step of introducing a reporter construct for detecting NR2F6 inhibition as disclosed herein into an ES-cell or a germ cell. The non-human transgenic animal provided and described herein is particular useful in screening methods and pharmacological tests described herein above. In particular the non-human transgenic animal described herein may be employed in drug screening assays as well as in scientific and medical studies wherein antagonists/inhibitors of NR2F6 for the treatment of a disease related to an insufficient immune response are tracked, selected and/or isolated.

The transgenic/genetically engineered cell(s), tissue(s), and/or non-human animals to be used in context of the present invention, in particular, the screening/identifying methods, preferably comprise the herein described and defined reporter constructs. Also in this context, reporter constructs may comprise a promoter and/or enhancer region of NR2F6 or of (a) component(s) of the NR2F6-dependent signalling pathway and a reporter as defined herein. Exemplary reporters (preferably associated with the reporter signals disclosed herein) are luciferase and fluorescent proteins, like GFP, RFP and the like. Exemplary, non-limiting constructs to be used may comprise a luciferase reporter under control of a (human) IL-17 promoter or a NF-AT/AP-1 NF-AT/AP-1 promoter and/or enhancer region. The use of such constructs in screening methods is also demonstrated in the appended examples. Exemplary reporters (preferably associated with the reporter signals disclosed herein) are luciferase and fluorescent proteins, like GFP, RFP and the like. These and other reporters/reporter constructs/reporter signals are also described herein above and below. The herein described (transgenic) cell(s), tissue(s) and/or non-human animals can also be used in accordance with the present invention (in particular the screening/identifying methods) in detecting whether the DNA binding of components of the NR2F6-dependent signalling cascade (e.g. NFAT/AP-1) is increased. For example, the binding of NFAT-AP-1 to the respective DNA enhancer sequence can be detected (e.g. by EMSA and the like).

In a further embodiment, the present invention relates to the use of a genetically engineered (transgenic) cell or a (transgenic) non-human animal for screening and/or validation of a compound suspected of being an antagonist of NR2F6. The term “cell” as used in this context may also comprise a plurality of cell as well as cells comprised in a tissue. A cell to be used may, for example any cells of the immune system. In a preferred embodiment of the present invention the used cells are special types of leukocytes, called lymphocytes, wherein the B and T cells are the major type of lymphocytes and are derived from hematopoietic stem cells in the bone marrow, in particular human cells/cell lines. For example, a high and stable expression of NR2F6 may facilitate the detection of a decrease in the NR2F6 activity. Since NR2F6 leads to a deactivation of components of the NR2F6-dependent signalling cascade as described above, a cell or a cell culture with a high NR2F6 expression will usually have a low activity and/or expression of said components of the signalling cascade. Accordingly, these cells are also highly useful in detecting an increase in the activity of said components. Since wild-type cells have sometimes a low or unstable NR2F6 expression, the use of transgenic (a) cell(s), tissue(s), non-human animal is particularly envisaged, if these cells have a high NR2F6 expression (reflected in a high protein or mRNA level). (Transgenic) cells(s), tissue(s) and non-human animals to be used in accordance with the present invention are also described herein above.

The used non-human animal or cell may be transgenic or non transgenic. In this context the term “transgenic” particularly means that at least one of the NR2F6 gene as described herein is overexpressed, thus the NR2F6 activity in the non-human transgenic animal or a transgenic animal cell is enhanced. Generally, it is preferred herein that NR2F6 is highly expressed in (a) cell(s), tissue(s), non-human animal to be used in the screening methods as described above.

The term “transgenic non-human-animal”, “transgenic cell” or “transgenic tissue” as used herein refers to an non-human animal, tissue or cell, not being a human that comprises different genetic material of a corresponding wild-type animal, tissue or cell. The term “genetic material” in this context may be any kind of a nucleic acid molecule, or analogues thereof, for example a nucleic acid molecule, or analogues thereof as defined herein. The term “different” means that additional or fewer genetic material in comparison to the genome of the wild type animal or animal cell. An overview of different expression systems to be used for generating transgenic cell/animal refers for example to Methods in Enzymology 153 (1987), 385-516, in Bitter et al (Methods in Enzymology 153 (1987), 516-544) and in Sawers et al. (Applied Microbiology and Biotechnology 46 (1996), 1-9), Billman-Jacobe (Current Opinion in Biotechnology 7 (1996), 500-4), Hockney (Trends in Biotechnology 12 (1994), 456-463), Griffiths et al., (Methods in Molecular Biology 75 (1997), 427-440).

In a preferred embodiment, the (transgenic) non-human animal or (transgenic) cell is or is derived from a mammal. Non-limiting examples of the (transgenic) non-human animal or derived (transgenic) cell are selected from the group consisting of a mouse, a rat, a rabbit, a guinea pig and Drosophila.

Generally, the (transgenic) cell may be a prokaryotic or eukaryotic cell. For example, the (transgenic) cell in accordance with the present invention may be but is not limited to bacterial, yeast, fungus, plant or animal cell. In general, the transformation or genetically engineering of a cell with a nucleic acid construct or a vector can be carried out by standard methods, as for instance described in Sambrook and Russell (2001), Molecular Cloning: A Laboratory Manual, CSH Press, Cold Spring Harbor, N.Y., USA; Methods in Yeast Genetics, A Laboratory Course Manual, Cold Spring Harbor Laboratory Press, 1990.

The invention also relates to a kit useful for carrying out the methods as described herein comprising the polynucleotides and/or antibodies capable of detecting the activity of NR2F6 as characterized above. The embodiments disclosed in this connection with the method of the present invention apply, mutatis mutandis, to the kit of the present invention.

Advantageously, the kit of the present invention further comprises, optionally (a) reaction buffers, storage solutions, wash solutions and/or remaining reagents or material required in the pharmacological and drug screening assays or the like as describes herein. Furthermore, parts of the kit of the invention can be packaged individually in vials or bottles or in combination in containers or multicontainer units.

In a preferred embodiment of the present invention the kit may be advantageously used for carrying out the method for detecting the NR2F6 activity or changes in the NR2F6-dependent signaling cascade as described herein. Additionally, the kit of the present invention may contain means for detection suitable for scientific, medical and/or diagnostic purposes. The manufacture of the kits follows preferably standard procedures which are known to the person skilled in the art.

Similarly, kits are provided which comprise the candidate molecule as described herein, the nucleic acid molecule, the vector, the cell, tissue or a non-human transgenic animal of the invention. These kits are provided herein are particularly useful in the methods of the present invention and in particular in the determination of the NR2F6 activity or changes in the NR2F6-dependent signaling cascade. These kits as well as the methods provided herein are also useful in pharmaceutical screenings, also comprising “high-throughput” screening. The technical advantage of the herein described methods as well as the kits is the use of NR2F6 or a fragment thereof as a functional biosensor.

In addition, the invention relates to a method of treating a disease related to an insufficient immune response, comprising administering an effective amount of an antagonist/inhibitor of NR2F6 to a patient in need thereof. Preferably, said patient is a human.

### FIGURES

FIG. 1: Genomic location and exon structure for human NR2F6.

FIG. 2: NR2F6 is a phosphoprotein and expressed in haematopoietic organs.

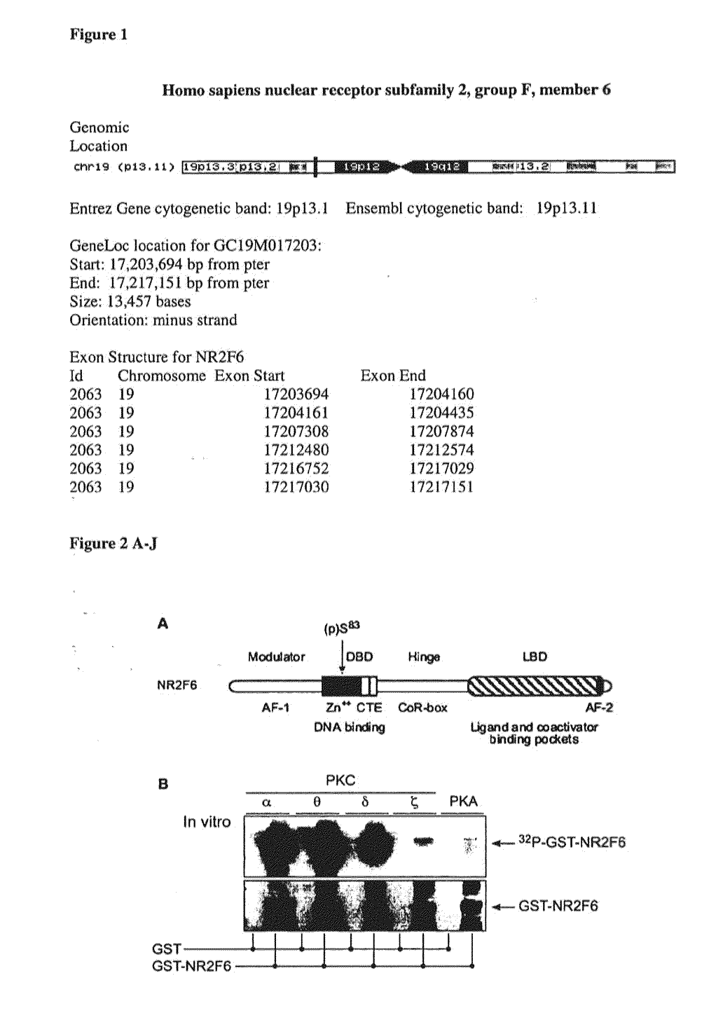

(A) Cartoon of NR2F6 protein structure modified after (Giguere (1999) Endocr Rev 20, 689-725; Moore (2006) Chem Med Chem 1, 504-523). (B & C) Protein kinase assays of distinct combinations of full-length GST-NR2F6 fusion proteins (wild-type, S83A and S89A mutants) incubated with recombinant members of the PKC family (alpha, theta, delta and zeta)) and Protein kinase A (PKA) in vitro. Identical amounts of GST-NR2F6 fusion proteins have been used per lane, as shown by the anti-GST immunoblot (lower panel). (D) GST pull down assays revealed that endogenous PKCtheta and PKCalpha co-precipitate with GST-NR2F6 (but not with PKB). (E) NR2F6 was immunoprecipitated from resting (−) or 20 min and 50 nM PDBu-stimulated (+) Jurkat cells, transfected with GFP control, NR2F6 wild-type or S83A, as indicated and immunoblotted with anti-(p)Ser-83 NR2F6 specific pAb. (F) Ser-83 phosphostatus on NR2F6 directly influenced DNA-binding to its TGACCT direct-repeat motif in nuclear extracts in EMSA analysis. Jurkat T cells were transfected with NR2F6 wild-type, S83A and S83E mutant, or GFP inert protein control, as indicated and were left unstimulated (−) or were stimulated with solid-phase CD3 and CD28 antibodies, as indicated. (G) The equal expression levels of the recombinant NR2F6 in nuclear fractions were confirmed by immunoblotting. (H & I) Expression of the low abundant NR2F6 mRNA in immune-relevant tissues. NR2F6 in situ hybridization was positive in thymus sections of wild-type (I) but not NR2F6^{-/-} (IV) E14.5 embryonic thymus sections. Expression of other COUP-TF family members NR2F1 (II, V) and NR2F2 (III, VI) were not altered in the NR2F6^{-/-} thymus. QRT PCR revealed NR2F6 expression in spleen, lymph node and bone marrow of wild-type but not NR2F6^{-/-} animals (I and not shown). (J) In primary CD3⁺ T cells, NR2F6 mRNA levels decreased upon 4 hrs stimulation with plate bound anti CD3 (10 μg/ml) and anti CD3 (10 μg/ml) plus co-stimulatory soluble anti CD28 (1 μg/ml). Data represent mean of two duplicates of 3 independent experiments. Error bars represent standard error.

FIG. 3: NR2F6^{-/-} lymphocytes hyperrespond to antigen receptor stimulation.

Proliferative (A & B) as well as IL-2 and IFNgamma cytokine secretion (D & E) responses of NR2F6-deficient CD3⁺, CD4⁺ and CD8⁺ splenic T cells were analyzed in comparison to wild-type littermate controls. After incubation with medium or different stimuli such as plate-bound anti-CD3, with or without soluble anti-CD28, or mitomycin C-treated splenocytes from BALB/c mice, cultures were analyzed using standard procedures. Data are representative of four independent experiments of duplicates, means are shown with error bars. (D) 2×2 ANOVA showed that under stimulating conditions NR2F6^{-/-} CD4⁺ T cells have significantly higher mean values than the wild-type p<0.000764, IFN-gamma secretion in CD8⁺ T cells was not significantly upregulated. (C) Flow cytometric analysis of CD3 expression was comparable between NR2F6 wild-type (^{+/+}) and knockout (^{-/-}) T cells. (F) Proliferation of purified splenic B cells stimulated with IgM (1.2 μg/ml) plus IL-4 (25 U/ml) for 72 h, analyzed by [³H]thymidine incorporation. Data of three independent experiments are shown plated in duplicates. p<0.01343. (G) Blood plasma concentrations of IL-2 cytokine secretion taken two hours after injection with SEB i.p. (10 μg/kg). IL-2 concentrations were measured by BioPlex technology. Data were analyzed with a welsh t-test; t=4.4982, df=14.722, p-value<0.0004442, n=10. (H) TCR-Vbeta8 surface expression was intact in NR2F6^{-/-} T cells, as shown by flow cytometry.

FIG. 4: NR2F6 acts as repressor of NF-AT/AP-1 DNA-binding capability.

(A) Purified CD3⁺ T cells (5×10⁶ per lane) from 6-week-old NR2F6^{+/+} and NR2F6^{-/-} mice were stimulated with CD3/CD28 for the indicated time periods. Proximal TCR signalling analysis was performed, phosphorylation status of indicated proteins were defined with antibodies to (p)Y-783 PLCgamma1, (p)S-32 IkappaBa and (p)ERK in comparison to total PLCgamma1 and total ERK were analyzed. One representative experiment of two is shown. DNA binding capacity of the NF-AT, AP-1 and NFkappaB consensus sequences in NR2F6^{+/+} and NR2F6^{-/-} CD3⁺ (B), CD4⁺ and CD8⁺ (C-E) T cell stimulated with CD3/CD28 in EMSA analysis are shown Supershift analysis was performed by using antibodies against fos (for the AP-1 EMSA) and NF-ATc (NF-AT EMSA) and p50 (for the NFkappaB EMSA). One representative experiment out of four is shown.

FIG. 5: NR2F6 deficiency causes late-onset hyperplasia and autoantibody production.

(A-E) Spleens of 12 month-old NR2F6^{-/-} mice were enlarged and percentages and total numbers of CD3⁺ and IgM/IgD⁺ lymphocytes were significantly increased in NR2F6^{-/-} mice. (B) Splenic weight, (C) total cellularity together with (D) CD3⁺ T cells and (E) mature B cell numbers are shown n=12-20. Statistical analysis was performed using a student t-test p values are (B) p<0.00872 (C) p<0.00648 (D) p<0.03315 (E) p<0.04655 error bars represent standard error. (F-I) 12 month-old NR2F6^{-/-} mice show elevated IgG1 plasma levels and production of autoantibodies against nuclear antigens (ANA) and double stranded DNA (dsDNA), as determined by staining of rat liver sections or by ELISA. (F) Increased serum IgG1 levels in 6-10 week and 12 month-old wild-type and NR2F6^{-/-} serum samples; p<0.007287. Statistic was calculated via student t-test n=9.

FIG. 6: Enhanced susceptible of NR2F6^{-/-} mice to Th17-dependent experimental autoimmune encephalomyelitis (EAE).

(A) Age-matched female mice immunized with MOG₃₅₋₅₅ peptide emulsified in CFA, followed by injection of pertussis toxin after 24 and 72 hrs. Mouse disease scores n=20, p values were calculated by comparing the mean values of wild-type and NR2F6^{-/-} scores at the onset of disease (d7-d13 p<0.000416) and disease progression (d14-d20 p<0.0249) via a Welch two sample t-test. (B) Production of IL-17 and (C) IFNgamma in recall assays of splenocytes from MOG₃₅₋₅₅-immunized mice restimulated with MOG₃₅₋₅₅ peptide for 72 hrs in vitro is significantly enhanced in the knockout situation. Data was analyzed with ANOVA (B) p<0.0001876 (C) p<0.00158. (D) NR2F6 mRNA expression decreased in effector/memory T cells of wild-type animals that had been exposed to EAE inducing MOG₃₅₅₅ immunization and restimulation with MOG₃₅₋₅₅ in vitro. Data represent the mean of duplicates of 3 independent experiments plus standard error.

FIG. 7: Cartoon of our working hypothesis.

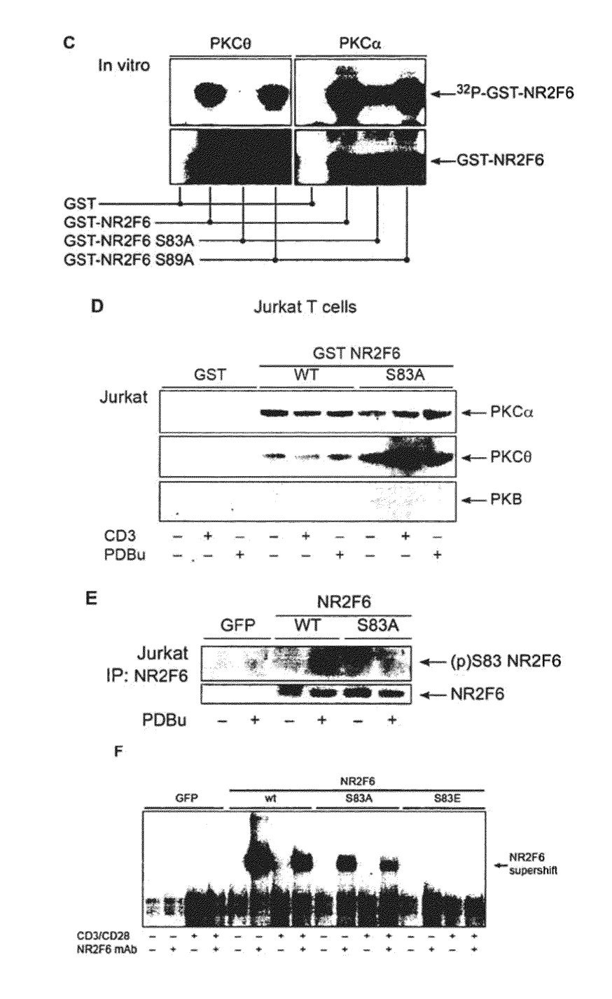

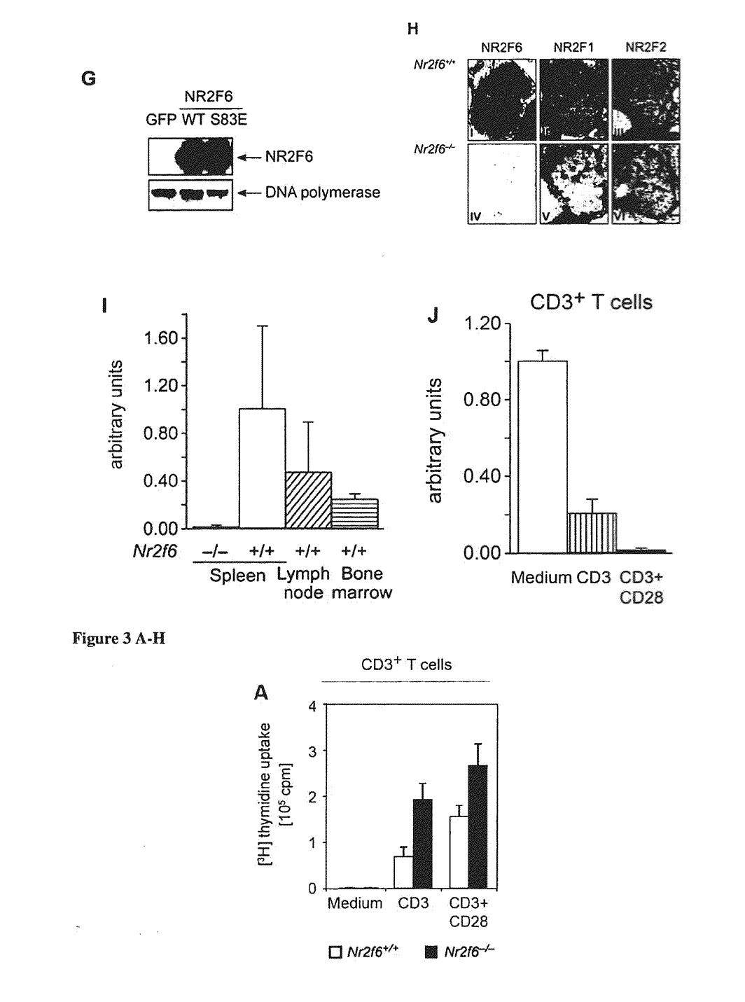

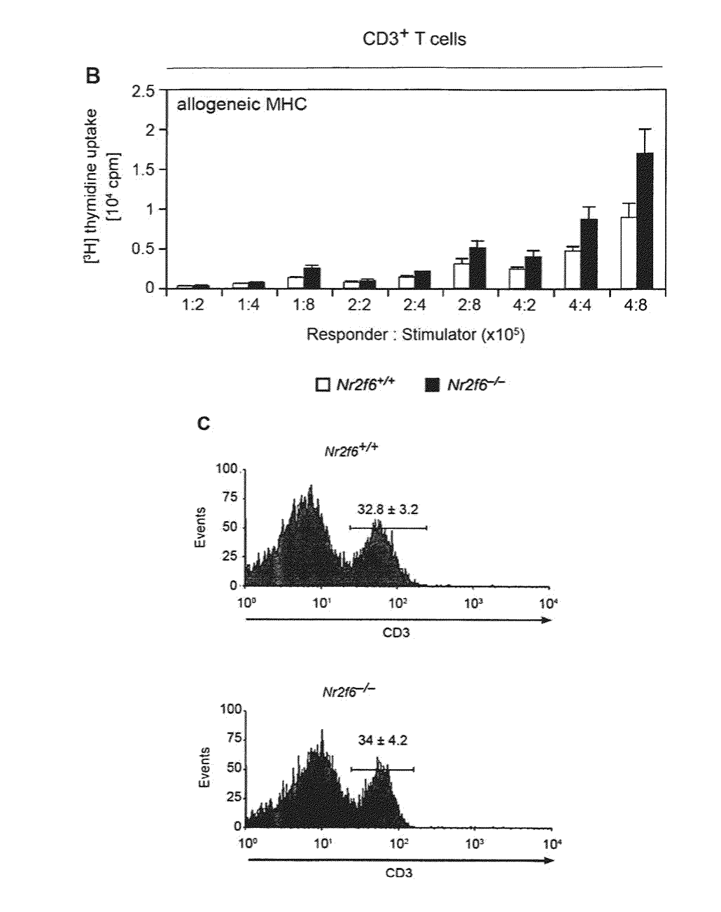

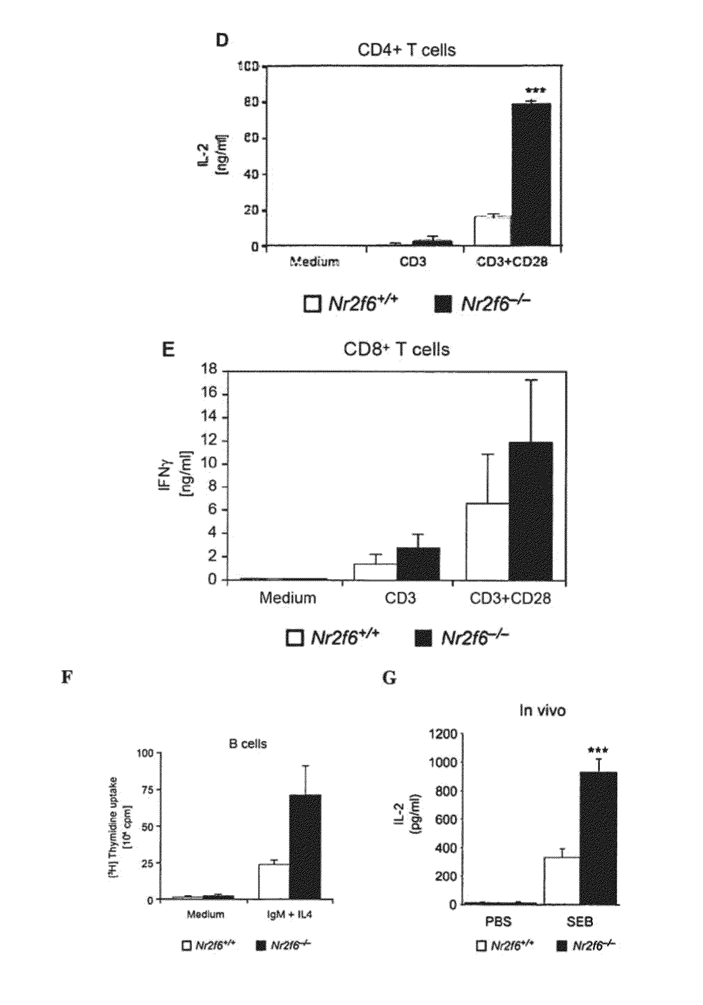

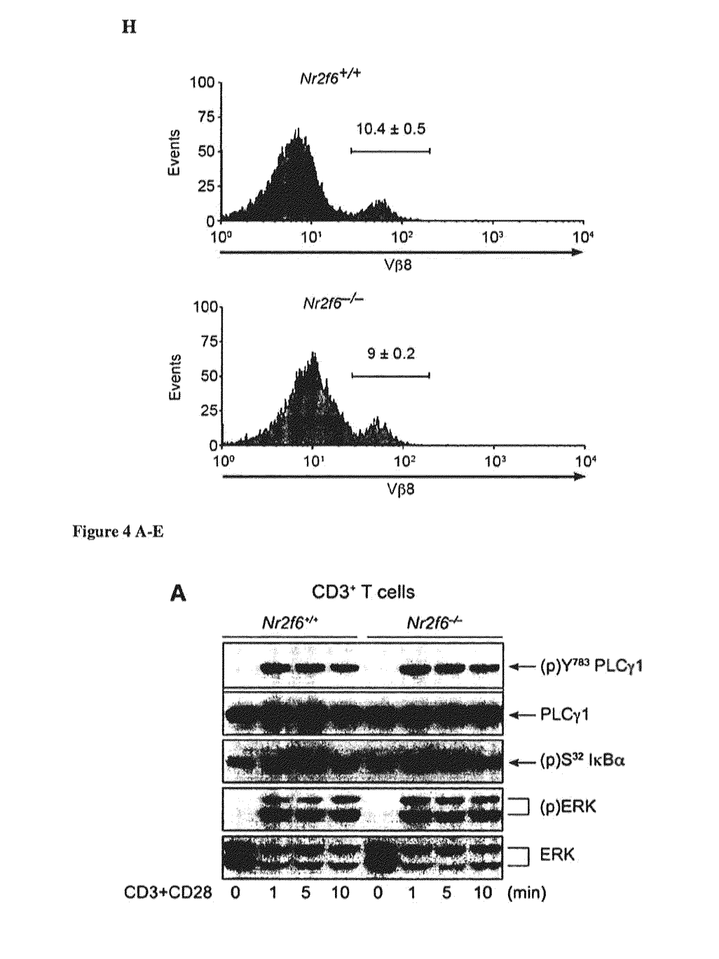

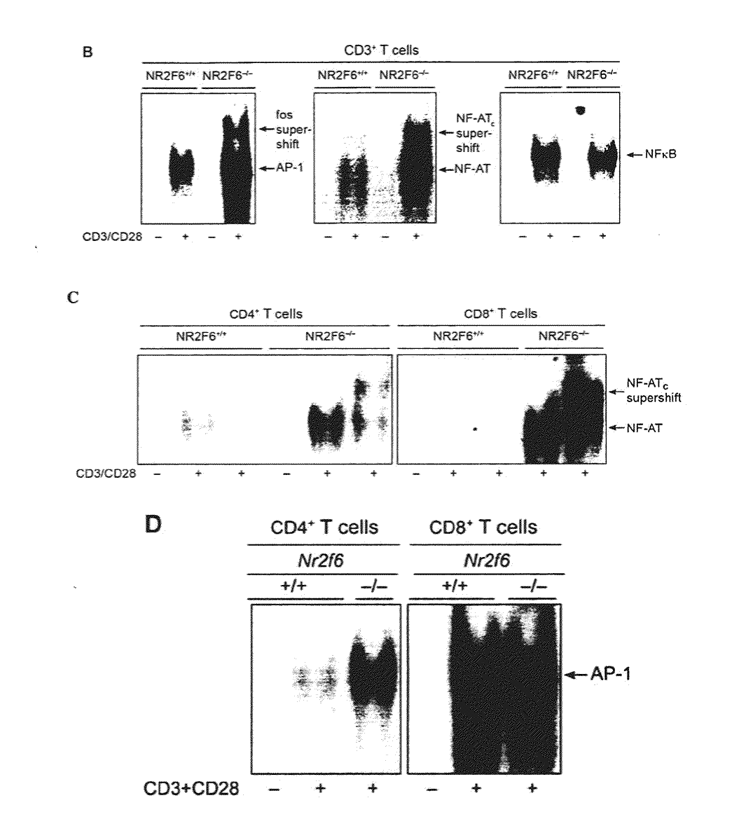

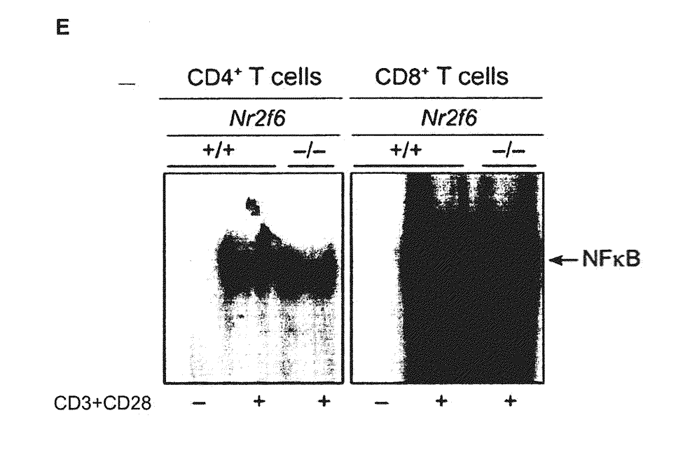

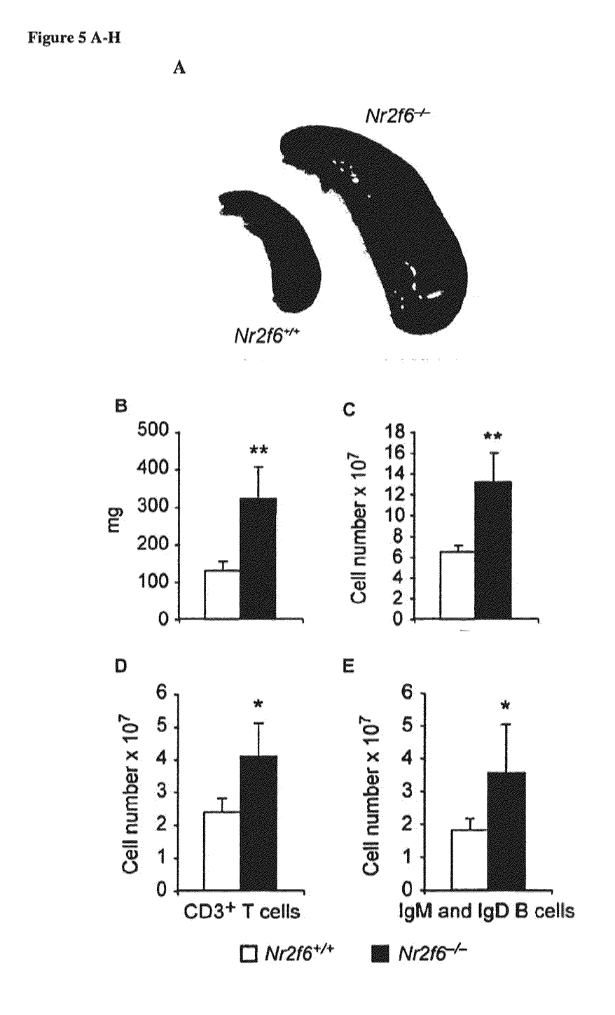

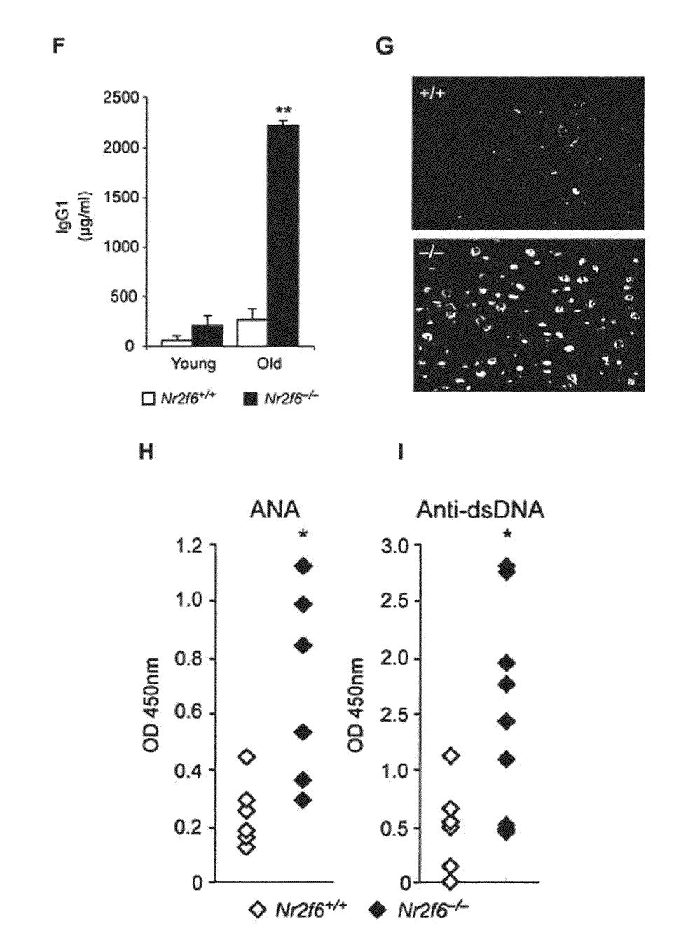

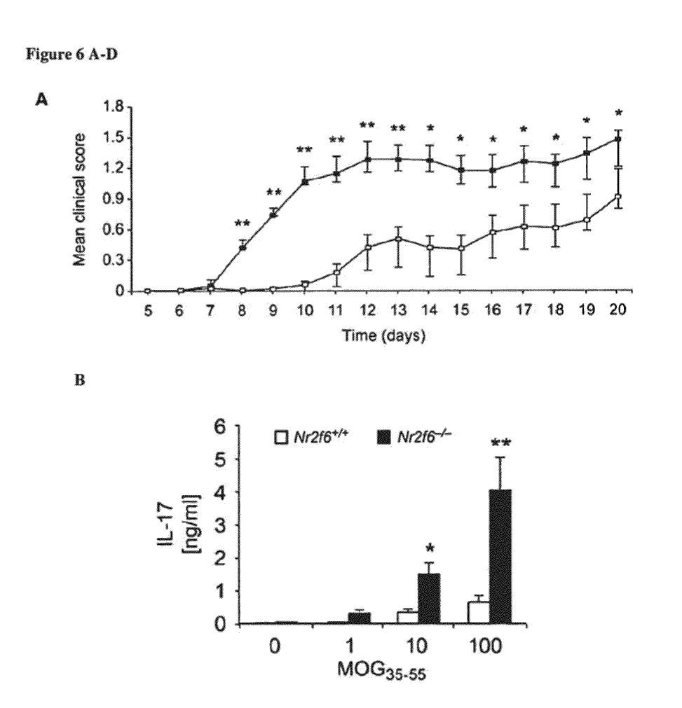

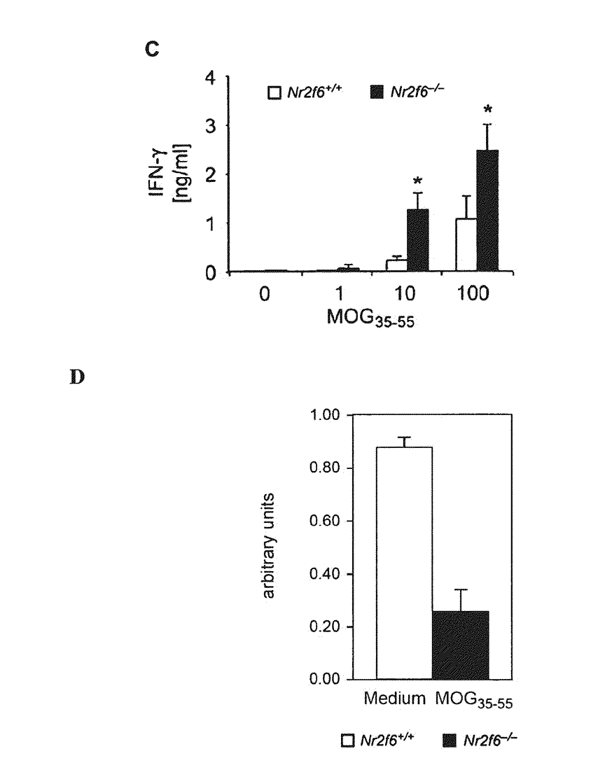

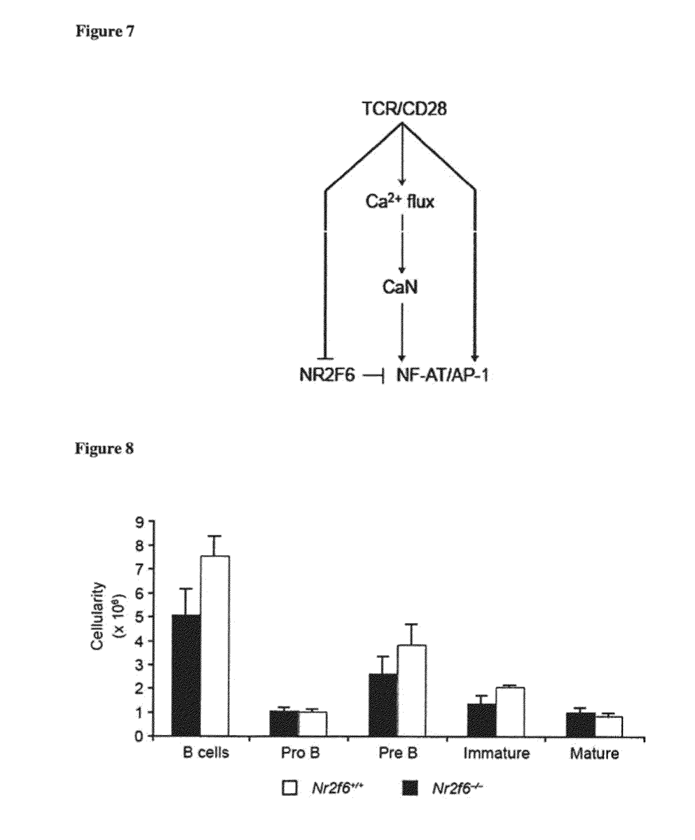

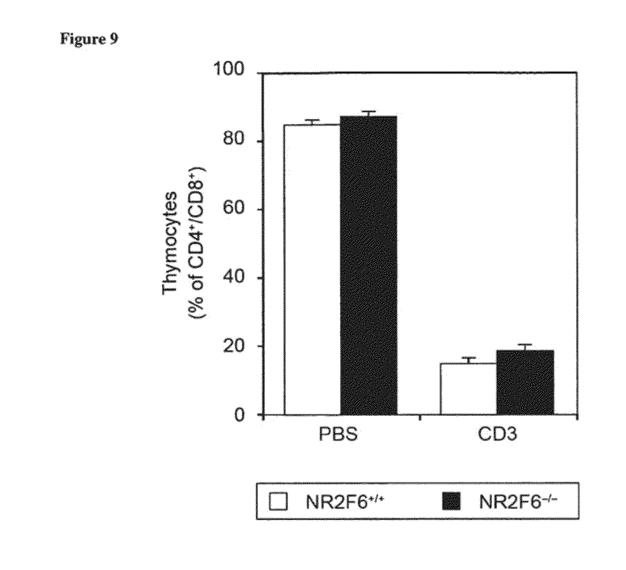

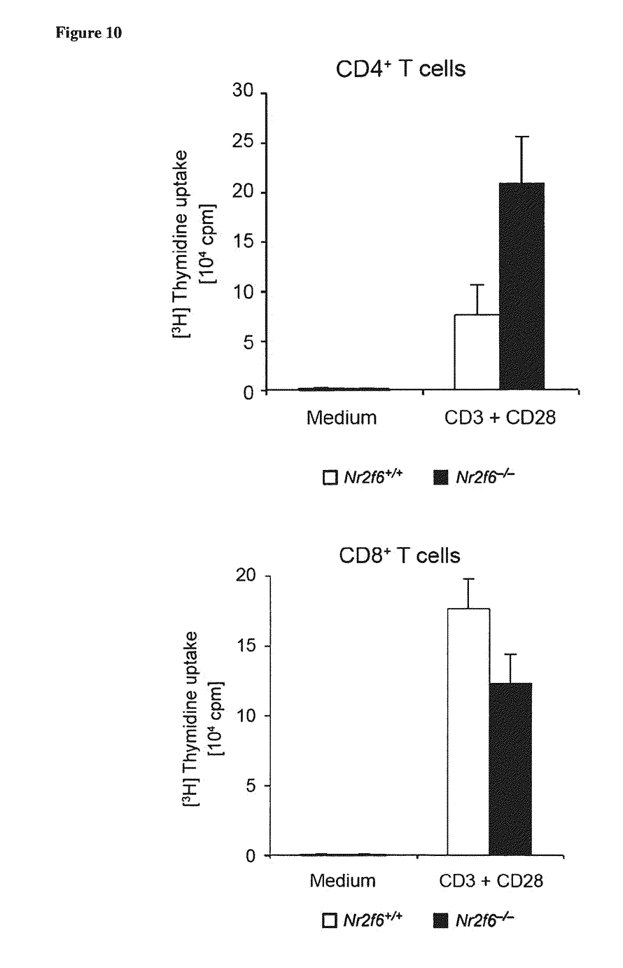

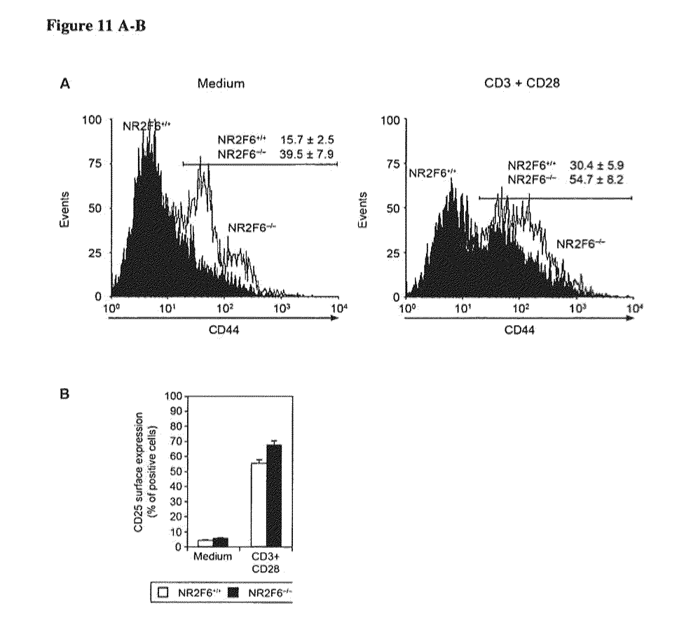

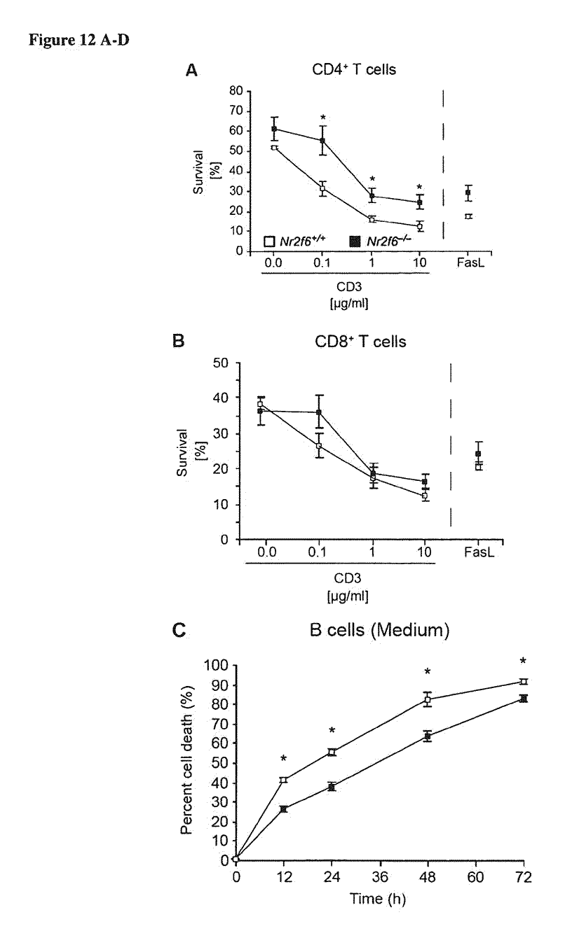

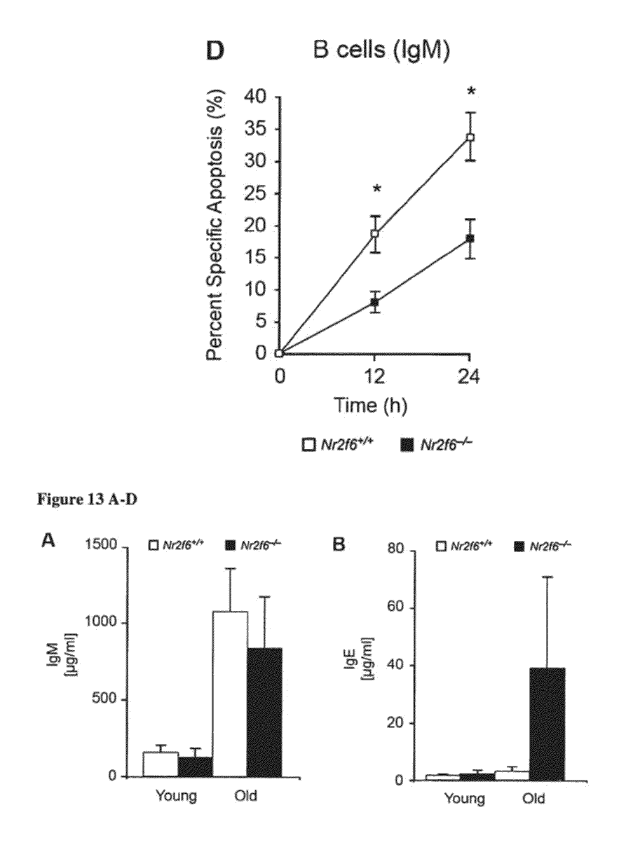

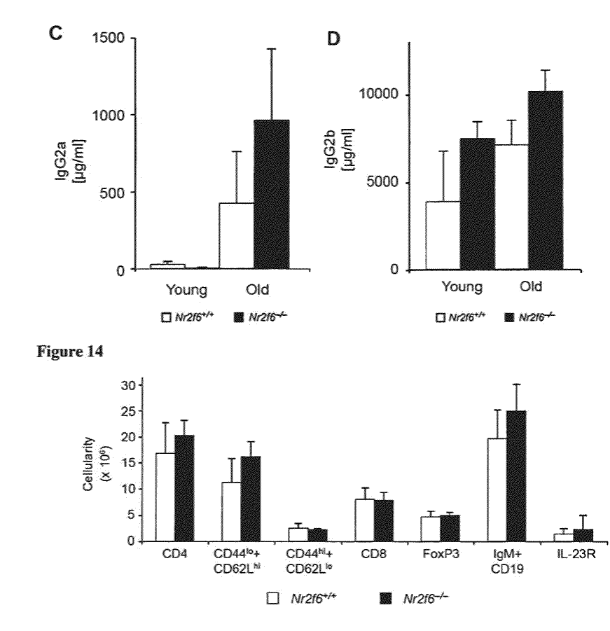

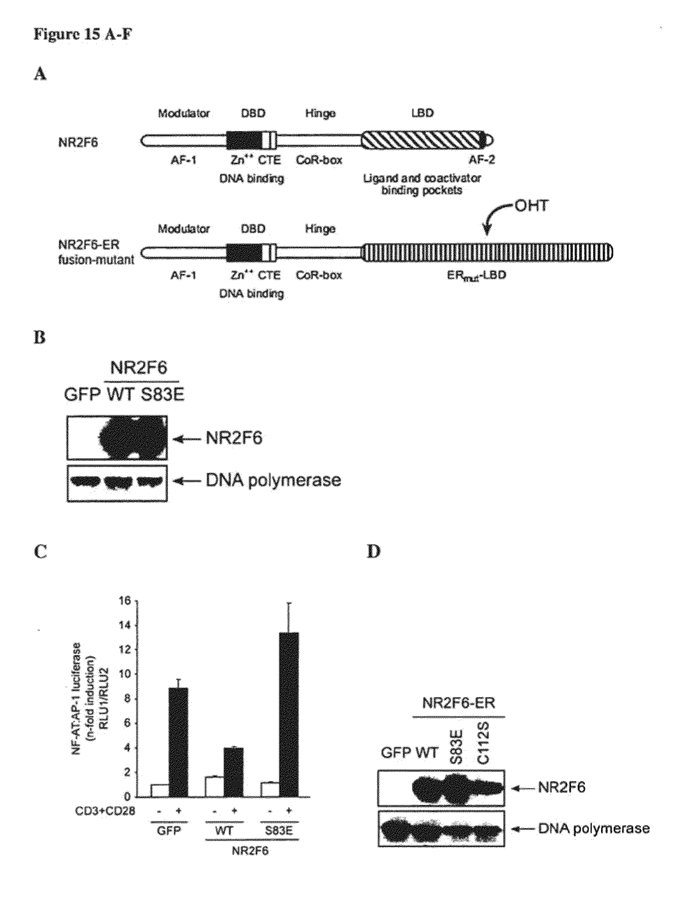

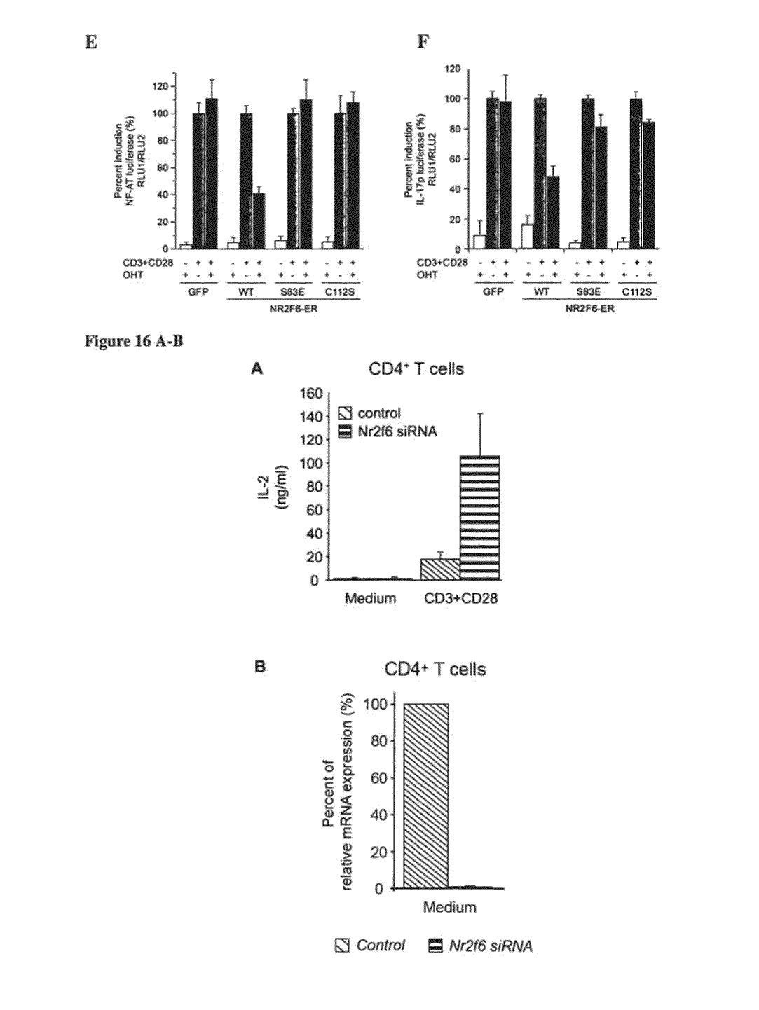

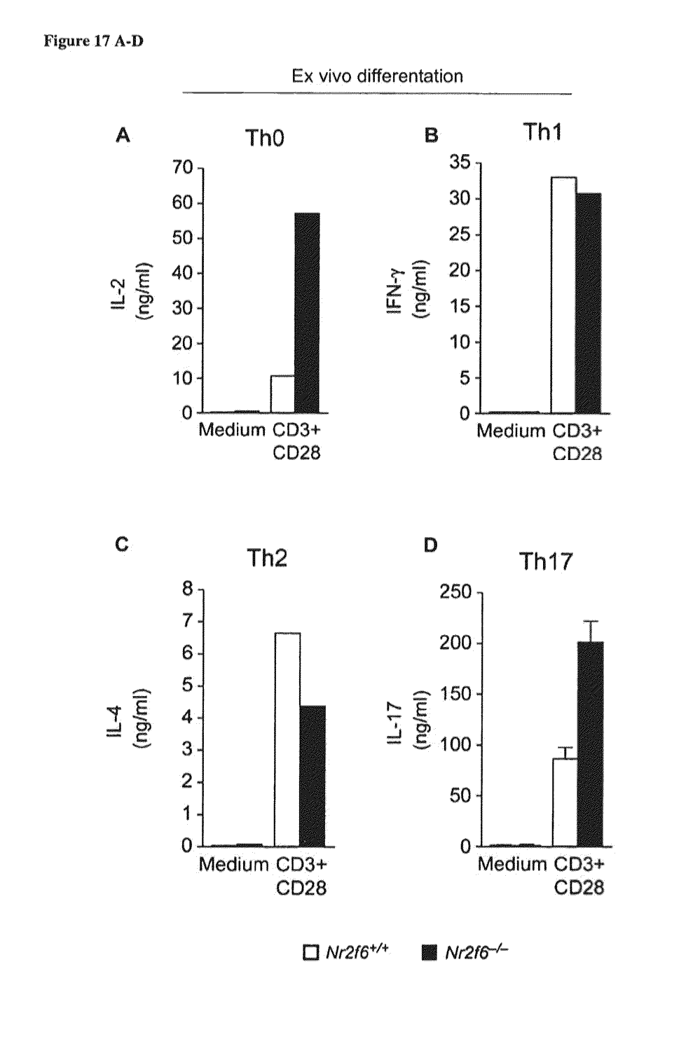

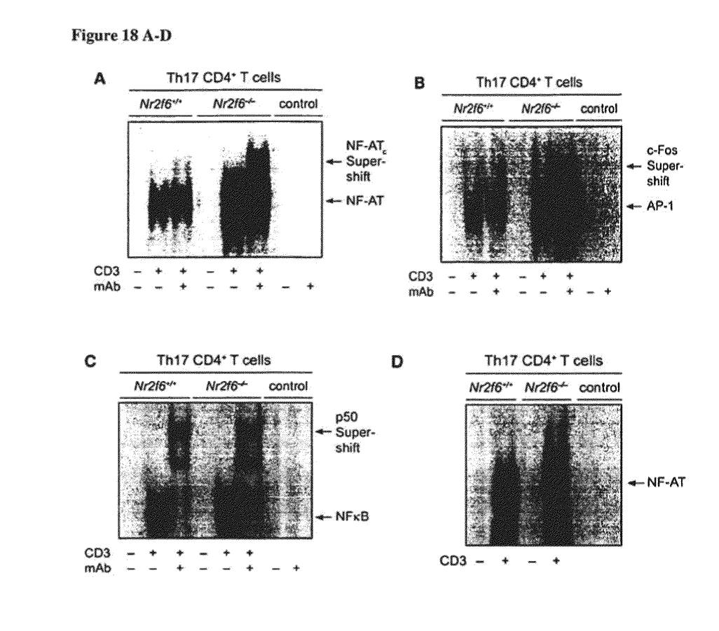

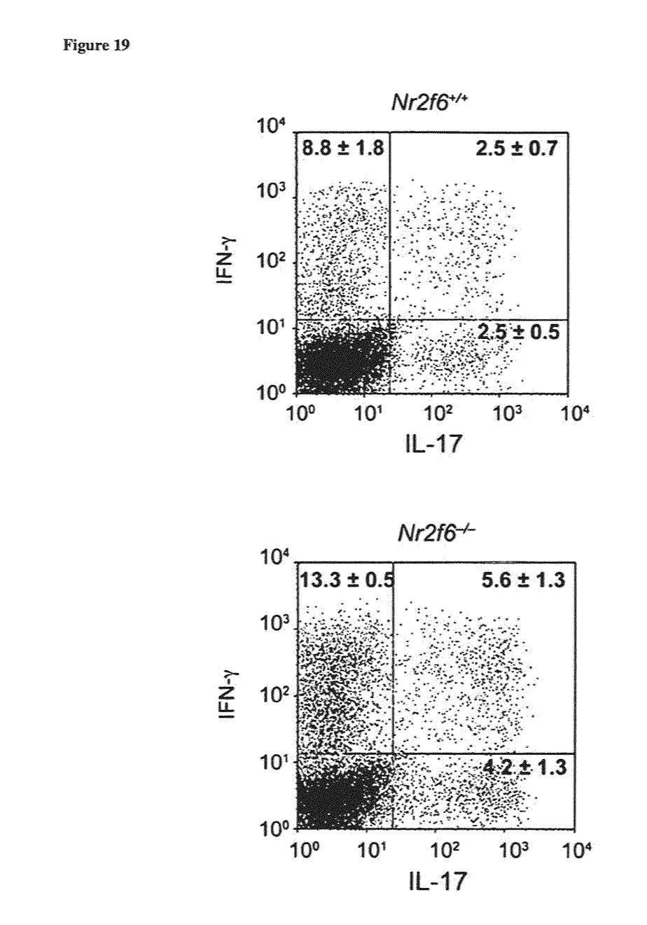

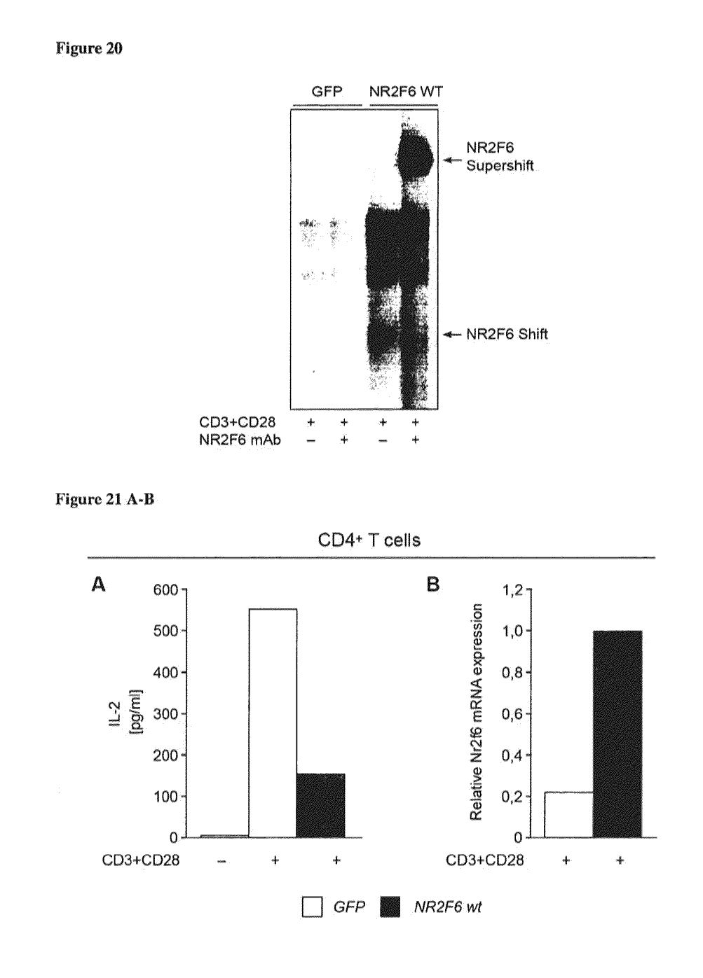

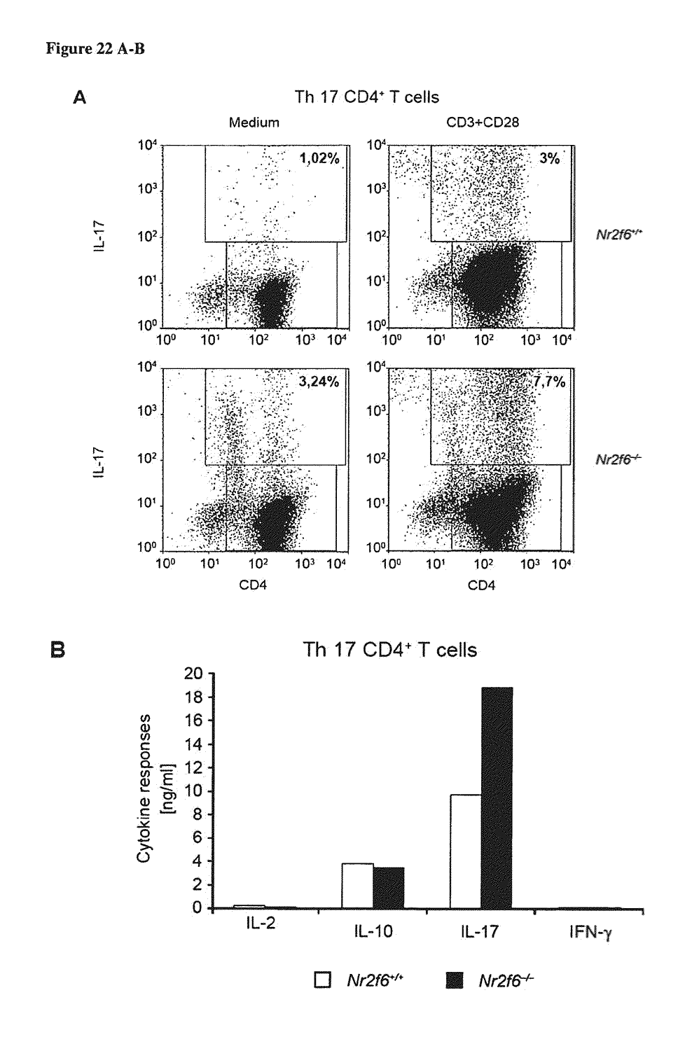

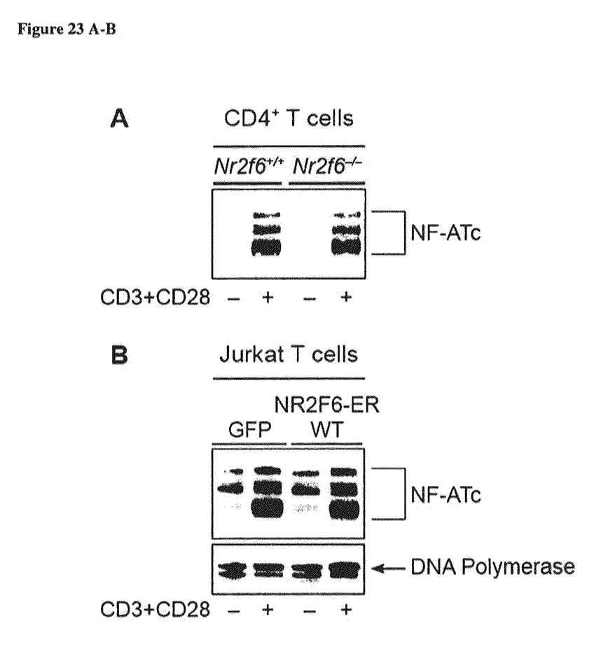

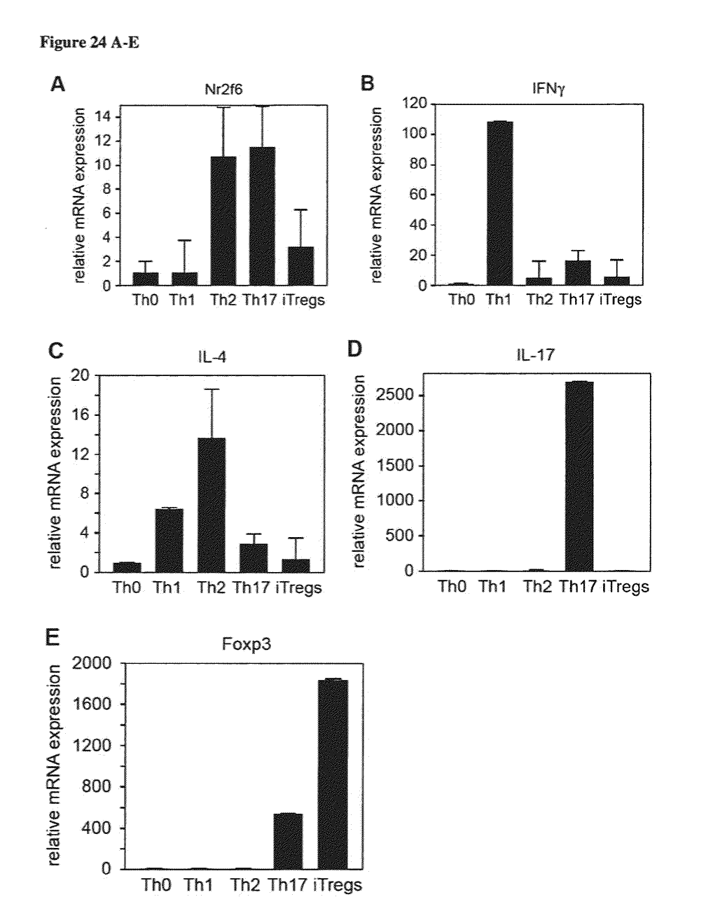

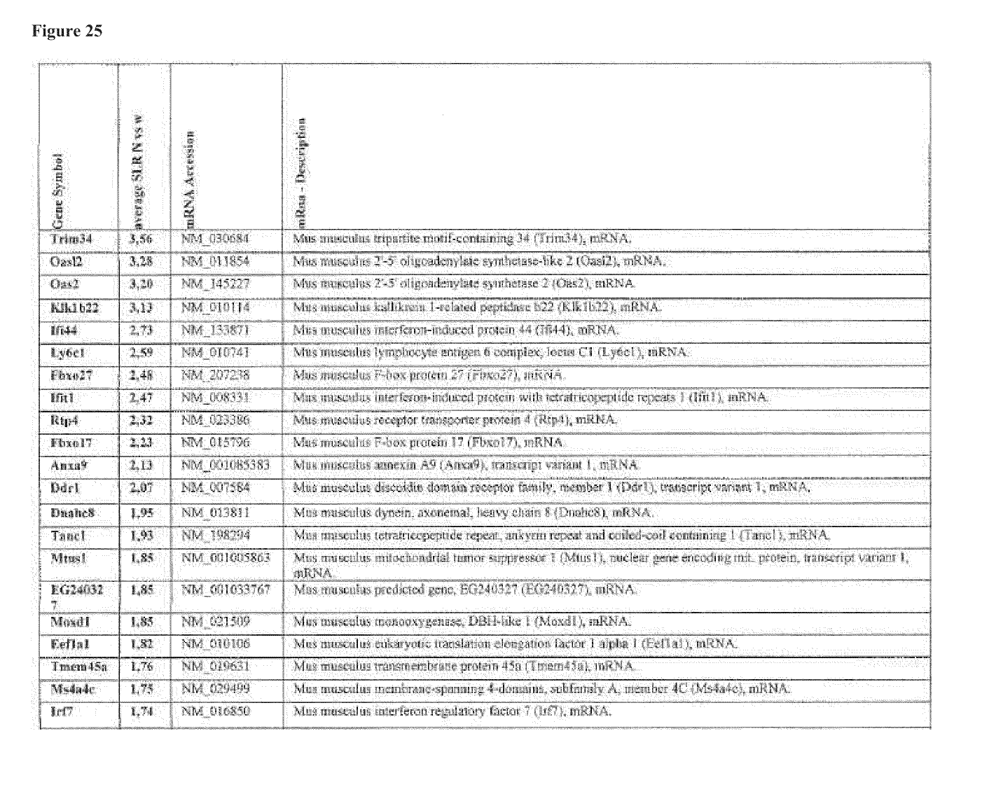

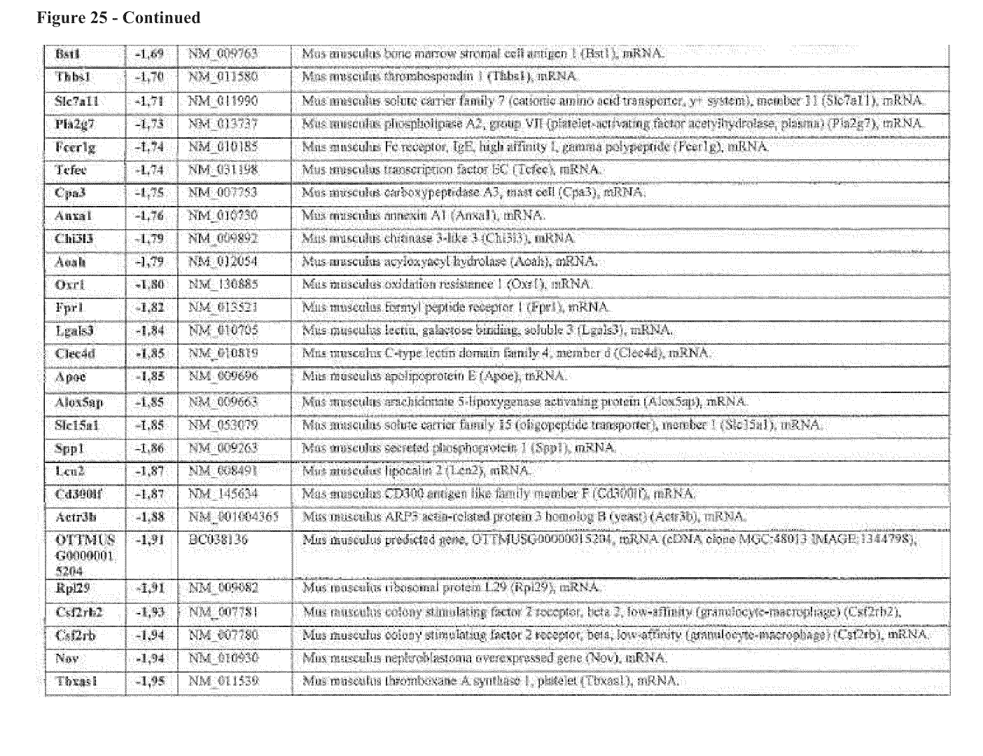

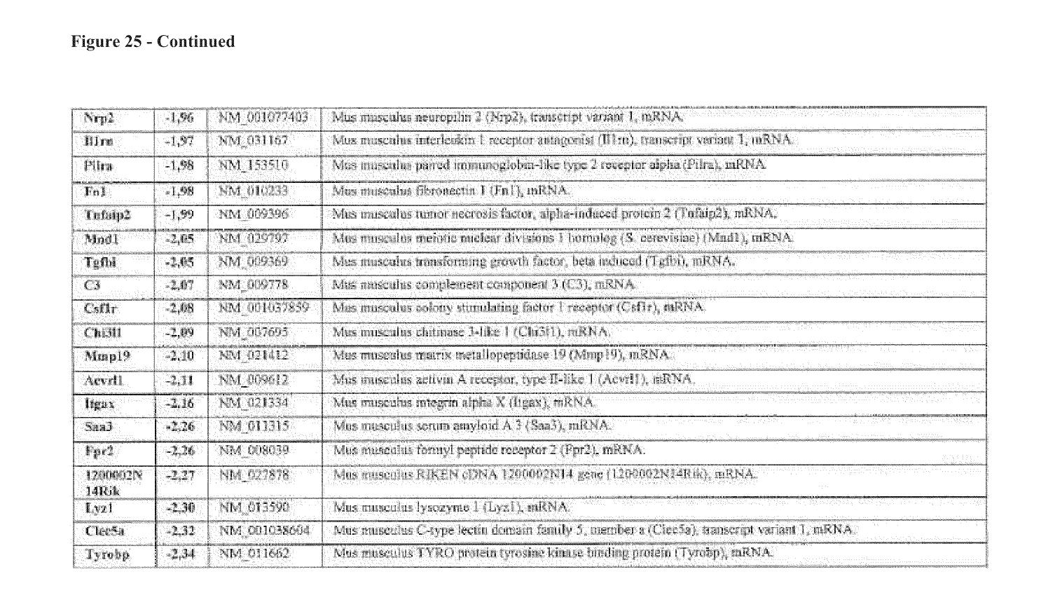

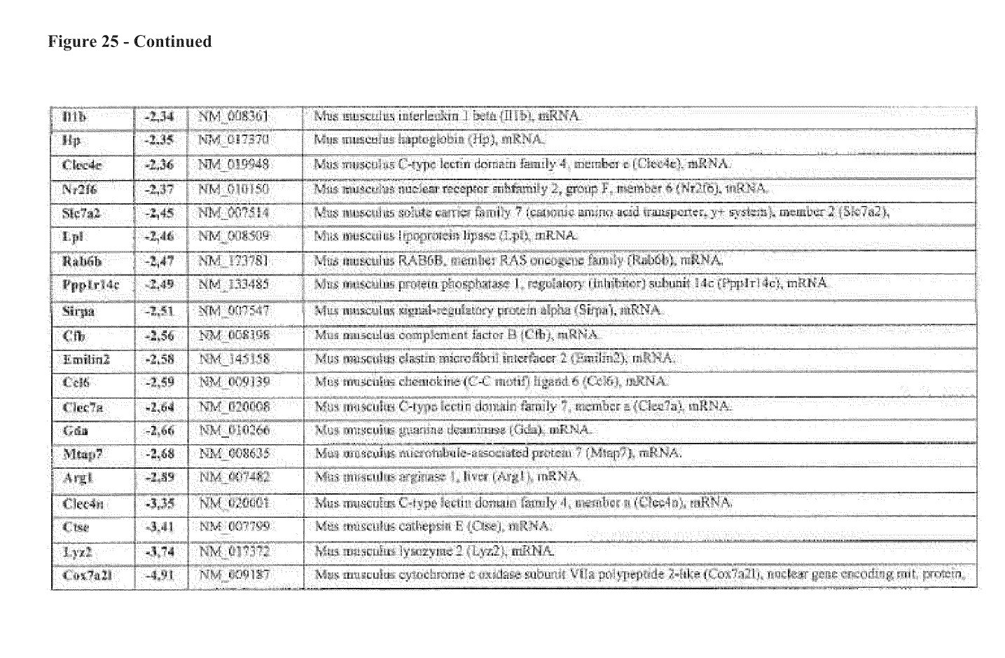

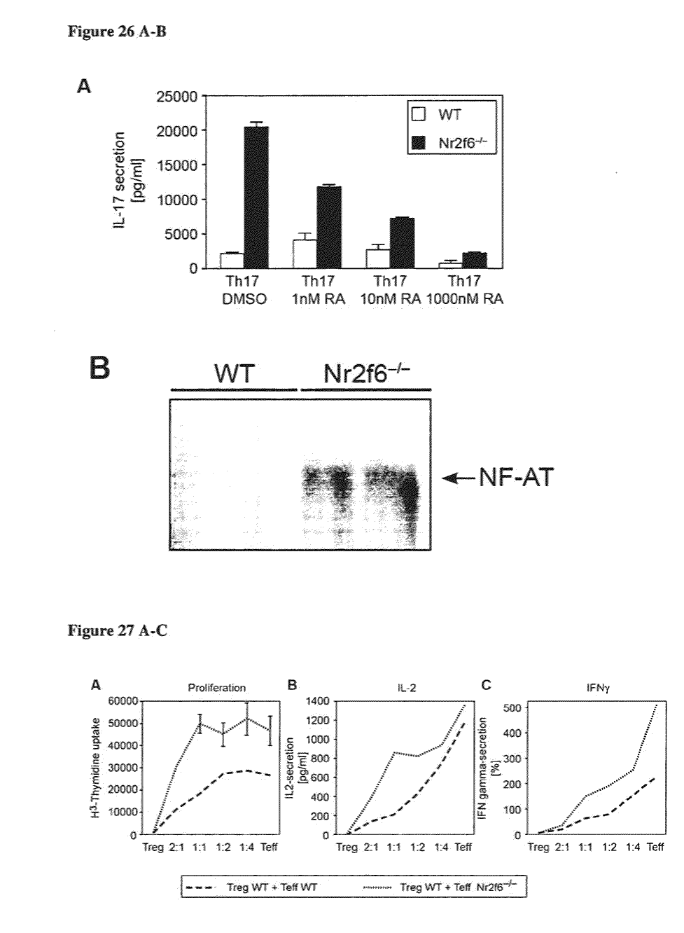

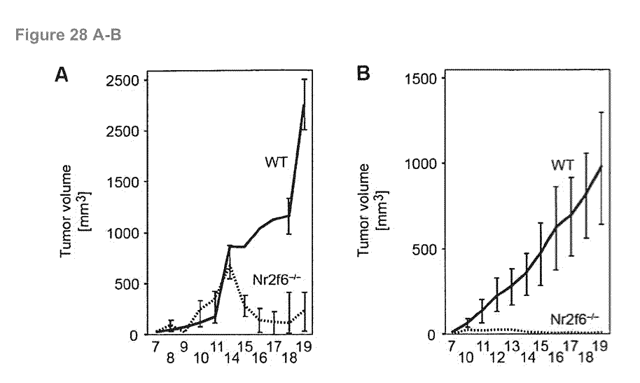

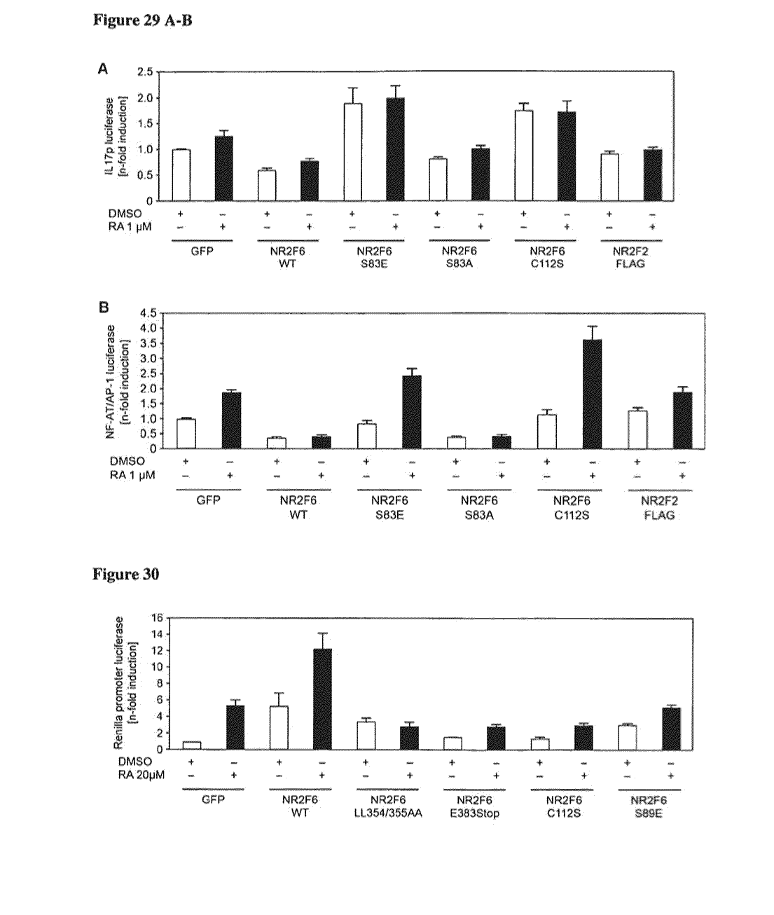

NR2F6 acts as repressor of DNA binding of nuclear factor of activated T cell (NF-AT)/activation protein 1 (AP-1). This is under the direct control of antigen receptor signalling, abrogating both mRNA level (see FIGS. 2J & 6D) and DNA binding capability (see FIG. 2F) of NR2F6.

FIG. 8: B cell development in the bone marrow.

B cell development in the bone marrow of NR2F6-deficient and NR2F6 wild-type littermate controls (n=3).

FIG. 9: CD3-induced depletion of thymocytes in vivo.

Negative selection was induced by anti-CD3 clone 2C11 injection (50 μg.) i.p. and analysis for double positive thymocytes in NR2F6-deficient and NR2F6 wild-type littermate controls was performed after 24 hrs (n=3).

FIG. 10: Proliferation responses of CD4⁺ and CD8⁺ T cells in vitro Proliferative responses of NR2F6-deficient and NR2F6 wild-type CD4⁺ and CD8⁺ splenic T cells were analyzed in comparison to wild-type littermate controls. After incubation with medium or different stimuli such as plate-bound anti-CD3, with or without soluble anti-CD28, cultures were harvested on filters at 50 or 64 hrs after a 16 hrs pulse with [³H]thymidine (1 μCi/well) and incorporation of [³H]thymidine was measured with a Matrix 96 direct betacounter system. Results shown are the means±SD of at least three independent experiments. Data are representative of three independent experiments of duplicates, means are shown with error bars.

FIG. 11: Flow cytometric analysis of expression of CD44 and CD25 on NR2F6^{+/+} and NR2F6^{-/-} T cells ex vivo.

Single-cell suspensions of purified peripheral CD3⁺ T cells, stimulated or not for 16 hr with anti-CD3 and anti-CD28, were stained with anti-CD25 and anti-CD44. Percentages of positive cells are indicated. (A) Splenic T cells expressed higher levels of CD44 in vivo, indicating an higher activation state. However, the kinetics and surface expression of CD25 (B), ICOS and CD69 (data not shown) were comparable between NR2F6^{+/+} and NR2F6^{-/-} T cells in response to CD31CD28 stimulation. The difference in surface CD44 expression was especially strinking in unstimulated cells t-test: t=3.2, df=11.2, p-value <0.008155, but is still significant after CD3/CD28 stimulation t=2.4, df=14.4, p-value <0.02620.

FIG. 12: Apoptotic rates of NR2F6^{-/-} T cells and B cells upon antigen receptor ligation in vitro.

(A) CD4⁺ T cells of NR2F6^{-/-} have a lower spontaneous apoptosis rate when induced with different concentrations of anti-CD3. (B) In contrast the apoptosis rate of the CD8⁺ T cell pool is intact when compared to wild-type controls. (C) Spontaneous and (D) IgM B cell apoptosis rate is also significant different compared to wild type controls. With CD4⁺ T cells a factorial split plot with the factors “genotypes”, with the six CD3 levels of each cell assembly of each animal revealed a significantly different p value p<0.02514 (n=6), that could not be found in the CD8⁺ T cells. B cells revealed following p values p<0.0364 (n=6), IgM p<0.0124.

FIG. 13: Plasma levels of IgM, IgE, IgG2a and IgG2b in older NR2F6^{-/-} mice.

Serum immunoglobulin levels of IgM, IgE, IgG2a and IgG2b of young (6-10 weeks) and aged (>12 month) NR2F6^{-/-} serum levels were not significant different compared to wild type controls. Experiments were repeated at least three times with similar results.

FIG. 14: NR2F6^{-/-} T and B cell subsets derived from EAE-disease affected mice.

No significant difference was seen in NR2F6^{-/-} T and B cell subsets when compared to wild-type NR2F6^{+/+} littermate controls.

FIG. 15: CD3/CD28-induced NF-AT/AP-1 composite element-dependent reporter is repressed by recombinant NR2F6. (A) Cartoon comparing structure of wild type NR2F6 protein and NR2F6-ER fusion mutant. (B) The equal expression levels of the recombinant NR2F6 in nuclear fractions were confirmed by immunoblotting (C) Transfected wild-type NR2F6 interfered with CD3 plus CD28-induced transcriptional activation of the NF-AT:AP-1 promoter luciferase reporter; this repressor activity was abolished by the S83E mutation in NR2F6. (D-F) The CD3/CD28-induced the NF-AT/AP-1 composite element-dependent reporter was repressed by recombinant NR2F6-ER in transiently transfected Jurkat T cells. (D) Cotransfection experiments in Jurkat T cells showed that recombinant NR2F6-ER wild-type but not DNA-binding-defective mutants, S83E and C112S, induced repression of CD3 plus CD28-induced NF-AT-dependent reporter luciferase gene transcription. (E) CD3 plus CD28-induced IL-17-dependent promoter luciferase reporter was similarly repressed by recombinant NR2F6-ER wild-type in transfected Jurkat T cells. Reporter induction rates were normalized for the transfected cells, and CD3 plus CD28-induced reporter activity without OHT treatment was arbitrarily set as 100%. Mean of at least two independent experiments analyzed in triplicates is shown. Error bars represent standard error. (F) Equal expression of NR2F6 wild-type and S83E and C112S mutant proteins in the nuclear fractions was confirmed by immunoblotting (DNA polymerase served as loading control).

This data from NR2F6 overexpression systems are complementary to data from Nr2f6-deficient T cells, thus demonstrating that NR2F6 is a nuclear attenuator that directly interferes with DNA-binding of NF-AT and, subsequently, transcriptional activity of i.e. the NF-AT:AP-1-dependent IL-17 gene expression.

FIG. 16: siRNA transfection

(A) Similarly to knock-out cells, siRNA-mediated Nr2f6 knockdown in CD4+ T cells resulted in enhanced IL-2 cytokine secretion upon CD3 plus CD28 stimulation, compared to siRNA nontargeting controls (n=2). (B) siRNA-mediated knock down abrogates target Nr2f6 mRNA levels qRT-PCR analysis of Nr2f6 mRNA level revealed that target Nr2f6 mRNA expression was diminished by >90% in Nr2f6 siRNA but not in the control siRNA bulk transfected CD4+ T cells. One of two representative experiments is shown.

FIG. 17: Naive CD4+ T cells differentiated under neutral Th0, Th1, Th2, and Th17 conditions.

Naive CD4+ T cells were differentiated under neutral Th0 (A), (B) Th1, (C) Th2, and (D) Th17 conditions, and relevant cytokines were measured from the supernatant after 4-5 days. One of two independent experiments with consistent results is shown for Th0, Th1, and Th2. IL-17 cytokine secretion in Nr2f6-deficient Th17 cells (black bars) was significantly higher than wild-type controls (white bars) (split-plot ANOVA Nr2f6+/+, n=5; Nr2f6−/−, n=5; p=0.004162) Error bars represent standard error.

FIG. 18: NR2F6 Suppresses NF-AT:AP-1 DNA Binding Specifically in CD4+ Th17 Effector-Memory T Cells

(A-C) EMSA analysis of nuclear extracts prepared from Th17-differentiated and aCD3-antibody-restimulated cells (IL-23, TGF-beta, IL-6, anti-IL-4, anti-IFN-gamma). NF-AT:AP-1 DNA binding was higher in Nr2f6-deficient extract when the NF-AT:AP-1 derived from the minimal IL-2 promoter was used, whereas NF-kappaB remained unchanged. (D) To distinguish between NF-AT:AP-1 and NF-AT-only binding, we used the NF-AT-specific probe #3 derived from the IL17A minimal promoter region (Liu et al., 2004); this again revealed a higher NF-AT binding in the Nr2f6-deficient nuclear Th17 cell extracts when compared to the wild-type control. Supershift analysis was performed with antibodies against c-fos, NF-ATc, and p50 as indicated. Controls are the radiolabeled probe with or without the supershifting Ab. One representative experiment out of two is shown.

FIG. 19: Cyokine production by CD4+ T cells isolated from CNS mononuclear cells 14 days after disease induction.

Cells were stimulated for 4 hr with PDBu plus ionomycin in the presence of Golgi stop and analyzed for IFN-gamma and IL-17 expression. A representative profile and mean±SD of cytokine staining are shown. A significant increase of IL-17-IFN-gamma double-positive cells (Nr2f6+/+, n=7; Nr2f6−/− n=7; p=0.0494) could be observed.

FIG. 20: Direct DNA Binding of NR2F6 as determined by EMSA analysis.

EMSA analysis employing transient Jurkat transfections has confirmed that only a slight shift can be observed but supershifting mAb (directed against an epitope with amino acids 13-44 of NR2F6) reproducibly induced a conformational switch and/or increased avidity that stabilizes NR2F6 DNA-binding. We did not see this stabilization effect with nonspecific antibody controls. The free probe does not shift with this NR2F6 mAb, as demonstrated in the GFP control transfections.

FIG. 21: NR2F6 overexpression suppresses IL-2 secretion in CD4+ T cells

(A) NR2F6 wild-type plasmid transfections of CD4+ T cells suppresses IL-2 secretion of CD3/CD28 stimulated cells after 24 h. One representative experiment out of two is shown. (B) qRT-PCR of Nr2f6 was used to control the transfection rate and data were normalized to GAPDH.

FIG. 22: IL-17 secretion in Th17 wild-type and Nr2f6-deficient cells

(A) Naïve CD4+ T cells from Nr2f6+/+ or Nr2f6−/− mice were differentiated under neutral (Medium) or stimulating (CD3 plus CD28) Th17 conditions for 4 days. IL-17 expressing cells were measured by intracellular staining after a 5 h PDBu plus lonomycin pulse in the presence of Golgi-plug. One representative experiment out of three is shown. These single cell level measurements of IL-17 expression in Th17 cells by flow cytometry confirmed data of secreted IL-17 cytokine levels obtained by BioPlex multi-analyte technology (FIG. 17). (B) In the Th17 subset, NR2F6 function appeared specific to the effector/memory but not regulatory cells, since no effect of Nr2f6 deficiency on IL-10 expression levels was observed. IL-10 secretion of the regulatory Th17 subsets was the same in wild-type (white bars) and Nr2f6-deficient (black bars) Th17 cells. Th17 differentiation status was controlled by IL-2, IL-17 and IFN-gamma secretion responses. One representative experiment of two is shown.

FIG. 23: NF-AT translocation is unaltered by NR2F6

NF-ATc translocation into the nucleus in unstimulated (−) and CD3 plus CD28 stimulated (+) (A) mouse wild-type and Nr2f6-deficient CD4+ T cells as well as in (B) Jurkat cells transfected with NR2F6-ER plasmid (under OHT co-treatment) was neither changed between the different genotypes nor between different transfected Jurkat extracts. DNA polymerase was used as loading control. One representative experiment out of 3 is shown.

FIG. 24: NR2F6 demonstrates a selectively high expression in Th2 and Th17 CD4+ T cells (A) Nr2f6 demonstrates a selectively high expression in Th2 and Th17 CD4+ T cells, when compared to Th0, Th1 and iTreg CD4+ T cells. Nr2f6 expression in Th0, Th1, Th2, Th17 and iTreg differentiated CD4⁺ T cells, data was normalized based on GAPDH expression. (B-E) Control qRT PCRs are shown in order to validate the proper differentiation of the naïve CD4⁺ T cells into the distinct T helper subsets as Th1 (IFNgamma), the Th2 (IL-4), the Th17 (IL-17) and the iTreg (Foxp3), as indicated. Data of two independent experiments is shown with standard error.

FIG. 25: Target synexpression groups of NR2F6 in Th17 T cells.

Affymetrics microarray expression analysis of Th17 differentiated CD4⁺ T cells. Relative fold induction of Nr2f6−/− T cells is shown in comparison to wild-type controls.

FIG. 26: NR2F6 is required for efficient retinoic acid (RA)-mediated immunosuppression of Th17 cells.

(A): Nr2f6^{-/-} Th17 T cells remain substantially resistant to RA-mediated immunesuppression. Nr2f6−/− CD4⁺ T cells have a significantly altered sensitivity towards RA treatment (500 nM) during Th17 differentiation when compared to wild type cells. IL-17 secretion on day 4 from wt and Nr2f6^{-/-} Th17 differentiated cells treated with increasing amounts of retinoic acid (RA) is shown. Data of three independent experiments performed in duplicates is shown with standard error. (B): RA abrogates NFAT DNA binding in wild type but not (or at least substantially less) in Nr2f6^{-/-} Th17 cells. Thus a NR2F6 antagonist will induce (partial) resistance to RA while a NR2F6 agonist will augment RA-mediated immunosuppression. EMSA analysis of nuclear extracts prepared from Th17 differentiated (IL-23, TGF-beta, IL-6, anti-IL-4, anti-IFN-gammaCD4⁺ T cells. EMSA analysis of nuclear extracts prepared from Th17 differentiated CD4⁺ T cells in the presence of RA (500 nM) revealed that NF-AT:AP-1 DNA binding was abrogated in wild type but not Nr2f6−/− Th17 cell extracts. A representative experiment is shown. Retinoic acid (RA), as a Vitamin A metabolite, is established to form ligands for retinoic acid-related nuclear receptors (RAR) that play pleiotropic roles in various biological processes. RA is known to be as a key modulator of TGF-beta-driven immunesuppression, capable of suppressing the differentiation of Th17 cells and conversely promoting the generation of Foxp3+ iTreg cells. Thus NR2F6 and its functional ligands, once defined, play a key role in balancing pathogenic Th17 versus regulatory iTreg T cell numbers and/or functions; modulation of this TGF-beta/RA/RAR regulatory module via NR2F6 ligand represent an innovative way to control a functional immune system.

FIG. 27. Nr2f6^{-/-} eff/memory T cells (Teff) are partially resistant to regulatory T cell (Treg)-mediated immunosuppression.

In order to analyse the sensitivity of Nr2f6−/− CD4+ effector T cells, we analysed the potential of Tregs to suppress wild-type and Nr2f6 effector cells. Nr2f6^{-/-} effector memory CD4 T cells are partially resistant towards Treg suppression as shown on the proliferation (A), and IL-2 (B) as well as IFNgamma cytokine (C) secretion response. One representative experiments is shown. This result is consistent with hyperplasia and late-onset immunopathology of Nr2f6^{-/-} mice (Immunity 08 paper). Thus a functional NR2F6 agonist will make T cells hypersensitive to Tregs, while a functional NR2F6 antagonist will make T cells hypersensitive to TGF-beta stimulation.

FIG. 28. Spontaneous tumor rejection in NR2F6^{-/-} mice.

Kinetics of tumor cell growth after tumor challenge with (A) 250.000 EL-4 tumor cells or (B) 100.000 E.G7 tumor cells is drastically reduced in the Nr2f6^{-/-} NR2F6^{-/-} mice when compared to the wild type mice. Data of two independent experiments with n=5 per group are shown with standard error.

FIG. 29. Transrepression assays for a NR2F6 compound finding program

NR2F6 transiently overexpressing Jurkat cells show trans-repression of both the human IL-17A promoter: luciferase reporter as well as a RAR promoter: luciferase reporter. NR2F6 wt and the S83A mutant proteins (but not NR2F2 wildtype, the closest relative to NR2F6) act as transcriptional repressors. As critical controls DNA-binding deficient mutants such as S83E or C112S are not able to suppress CD3/CD28-induced promoter reporter transactivation.

FIG. 30. Transcriptional activity of NR2F6 appears ligand-dependent.

NR2F family members appear as (hydrophobic) ligand-activated receptors whose apparent constitutive activity in cells results from the binding to an endogenous ligand (Kruse et al., PLOS Biol, 2008). LBD of NR2F6 is evolutionary conserved and critical for its transcriptional activity as shown by site-directed mutagenesis of the LBD domain employing the critical Leu 354/355 to Ala double mutant and an AF-2 E383Stop truncation mutant of NR2F6

The present invention is additionally described by way of the following illustrative non-limiting Examples, that provide a better understanding of the present invention and of its many advantages.

The following examples illustrate the invention:

### EXAMPLES

### Example 1

### Generation of NR2F6 Deficient Mice

NR2F6 knockout mice on a 129/SV background were generated as described in Warnecke (2005) Genes & development 19; 614-625. Accordingly, the NR2F6 gene was disrupted in murine embryonic stem (ES) cells using homologous recombination. The first two coding exons (DNA-binding domain) were replaced by an IRESLacZ/MCIneo cassette using the pKOS/EAR2-39 target vector (Lexicon Genetics). Targeted ES clones were identified by Southern blotting, and a mouse line with a disrupted NR2F6 gene was established using standard techniques.

### Example 2

### Identification of NR2F6-Ser83 as a PKC phosphorylation Site

Employing a new PKC phosphorylation site prediction program (Fujii (2004) PNAS 101:13744-13749), we identified NR2F6 (see protein structure in FIG. 2A) as a prime PKC substrate candidate in silica. Due to the combined presence of favoured and the absence of disfavoured residues within the surrounding sequences, both Ser-83 and Ser-89 on human NR2F6 were predicted to be phosphorylated by PKC. Indeed, PKCalpha, delta and theta (but not PKCzeta or PKA) were able to phosphorylate recombinant NR2F6 in vitro (FIG. 2B) This phosphorylation was lost after the predicted phosphorylation site at Ser-83 (but not at Ser-89) was mutated in the full-length NR2F6 protein, indicating that the evolutionary highly conserved Ser-83 on NR2F6 is the major PKC phosphorylation site on NR2F6 (FIG. 2C). Consistently, endogenous PKCalpha and PKCtheta bound to recombinant NR2F6 in pull down assays from Jurkat T cell lysates, identifying NR2F6 as an interacting protein partner for these PKC isotypes (FIG. 2D). This physical interaction occurred independently of T cell stimulation and the Ser-83 phosphorylation status on NR2F6. The presence of the novel PKC phospho-site on NR2F6 was further confirmed using a phosphospecific (p)Ser-83 antiserum that reacted with wild-type recombinant NR2F6 but not with its corresponding S83A mutant in stimulated Jurkat T cells (FIG. 2E). Taken together, these data indicate that NR2F6 is a phospho-protein at its Ser-83 site and a potential PKC substrate in Jurkat T cells.

DNA binding of recombinant NR2F6 itself to the TGACCT direct-repeat motif was observed in electrophoretic mobility band-shift assays (EMSA) from nuclear extracts derived from unstimulated cells (FIG. 2F). CD3/CD28 stimulation reduced DNA-binding activity of wild-type NR2F6 in this EMSA analysis albeit protein expression levels of NR2F6 protein in nuclear extracts remained unaltered (not shown). Since Ser-83 on NR2F6 was found to be the major phosphorylation site of PKC isotypes and located within the DNA-binding domain, we investigated if the phosphorylation status on this site could be involved in the observed CD3/CD28 activation-induced inhibition of NR2F6 DNA-binding. In order to answer this question, Ser-83 neutral (S83A) and acidic exchange (S83E) mutant of NR2F6 proteins were expressed at comparable levels and analyzed in parallel (FIG. 2G)

The S83A mutant of NR2F6 (S83A-NR2F6) as used herein may be encoded by the following nucleic acid sequence (start codon in bold, mutated nucleic acids leading to S83A mutation are underlined):

| (SEQ ID NO: 5) | |
|---|---|
| 1 | gtgcagcccg tgccccccgc gcgccggggc cgaatgcgcg ccgcgtaggg tcccccgggc |
| 61 | cgagaggggt gcccggaggg aagagcgcgg tgggggcgcc ccggccccgc tgccctgggg |
| 121 | ctatggccat ggtgaccggc ggctggggcg gccccggcgg cgacacgaac ggcgtggaca |
| 181 | aggcgggcgg ctacccgcgc gcggccgagg acgactcggc ctcgcccccc ggtgccgcca |
| 241 | gcgacgccga gccgggcgac gaggagcggc cggggctgca ggtggactgc gtggtgtgcg |
| 301 | gggacaagtc gagcggcaag cattacggtg tcttcacctg cgagggctgc aagagctttt |
| 361 | tcaagcgagc catccgccgc aacctcagct acacctgccg gtccaaccgt gactgccaga |
| 421 | tcgaccagca ccaccggaac cagtgccagt actgccgtct caagaagtgc ttccgggtgg |
| 481 | gcatgaggaa ggaggcggtg cagcgcggcc gcatcccgca ctcgctgcct ggtgccgtgg |
| 541 | ccgcctcctc gggcagcccc ccgggctcgg cgctggcggc agtggcgagc ggcggagacc |
| 601 | tcttcccggg ggaggcggtg tccgaactga tcgcgcagct gctgcgcgct gagccctacc |
| 661 | ctgcggcggc cggacgcttc ggcgcagggg gcggcgcggc gggcgcggtg ctgggcatcg |
| 721 | acaacgtgtg cgagctggcg gcgcggctgc tcttcagcac cgtggagtgg gcgcgccacg |
| 781 | cgcccttctt ccccgagctg ccggtggccg accaggtggc gctgctgcgc ctgagctgga |
| 841 | gcgagctctt cgtgctgaac gcggcgcagg cggcgctgcc cctgcacacg gcgccgctac |
| 901 | tggccgccgc cggcctccac gccgcgccta tggccgccga gcgcgccgtg gctttcatgg |
| 961 | accaggtgcg cgccttccag gagcaggtgg acaagctggg ccgcctgcag gtcgactcgg |
| 1021 | ccgagtatgg ctgcctcaag gccatcgcgc tcttcacgcc cgacgcctgt ggcctctcag |
| 1081 | acccggccca cgttgagagc ctgcaggaga aggcgcaggt ggccctcacc gagtatgtgc |
| 1141 | gggcgcagta cccgtcccag ccccagcgct tcgggcgcct gctgctgcgg ctccccgccc |
| 1201 | tgcgcgcggt ccctgcctcc ctcatctccc agctgttctt catgcgcctg gtggggaaga |
| 1261 | cgcccattga gacactgatc agagacatgc tgctgtcggg gagtaccttc aactggccct |
| 1321 | acggctcggg ccagtgacca tgacggggcc acgtgtgctg tggccaggcc tgcagacaga |
| 1381 | cctcaaggga cagggaatgc tgaggcctcg aggggcctcc cggggcccag gactctggct |
| 1441 | tctctcctca gacttctatt ttttaaagac tgtgaaatgt ttgtcttttc tgttttttaa |
| 1501 | atgatcatga aaccaaaaag agactgatca tccaggcctc agcctcatcc tccccaggac |
| 1561 | ccctgtccag gatggagggt ccaatcctag gacagccttg ttcctcagca cccctagcat |
| 1621 | gaacttgtgg gatggtgggg ttggcttccc tggcatgatg gacaaaggcc tggcgtcggc |
| 1681 | cagaggggct gctccagtgg gcaggggtag ctagcgtgtg ccaggcagat cctctggaca |
| 1741 | cgtaacctat gtcagacact acatgatgac tcaaggccaa taataaagac atttcctacc |
| 1801 | tgca |

which corresponds to the following amino acid sequence (S83A mutation underlined):

(SEQ ID NO: 6)
MAMVTGGWGGPGGDTNGVDKAGGYPRAAEDDSASPPGAASDAEPGDEE
RPGLQVDCVVCGDKSSGKHYGVFTCEGCKSFFKRAIRRNLSYTCRSNR
DCQIDQHHRNQCQYCRLKKCFRVGMRKEAVQRGRIPHSLPGAVAASSG
SPPGSALAAVASGGDLFPGQPVSELIAQLLRAEPYPAAAGRFGAGGGA
AGAVLGIDNVCELAARLLFSTVEWARHAPFFPELPVADQVALLRLSWS
ELFVLNAAQAALPLHTAPLLAAAGLHAAPMAAERAVAFMDQVRAFQEQ
VDKLGRLQVDSAEYGCLKAIALFTPDACGLSDPAHVESLQEKAQVALT
EYVRAQYPSQPQRFGRLLLRLPALRAVPASLISQLFFMRLVGKTPIET
LIRDMLLSGSTFNWPYGSGQ

The S83E mutant of NR2F6 (S83E-NR2F6) as used herein may be encoded by the following nucleic acid sequence (start codon in bold, mutated nucleic acids leading to S83E mutation are underlined).

| (SEQ ID NO: 7) | |
|---|---|
| 1 | gtgcagcccg tgccccccgc gcgccggggc cgaatgcgcg ccgcgtaggg tcccccgggc |
| 61 | cgagaggggt gcccggaggg aagagcgcgg tgggggcgcc ccggccccgc tgccctgggg |
| 121 | ctatggccat ggtgaccggc ggctggggcg gccccggcgg cgacacgaac ggcgtggaca |
| 181 | aggcgggcgg ctacccgcgc gcggccgagg acgactcggc ctcgcccccc ggtgccgcca |
| 241 | gcgacgccga gccgggcgac gaggagcggc cggggctgca ggtggactgc gtggtgtgcg |
| 301 | gggacaagtc gagcggcaag cattacggtg tcttcacctg cgagggctgc aagagctttt |
| 361 | tcaagcgaga gatccgccgc aacctcagct acacctgccg gtccaaccgt gactgccaga |
| 421 | tcgaccagca ccaccggaac cagtgccagt actgccgtct caagaagtgc ttccgggtgg |
| 481 | gcatgaggaa ggaggcggtg cagcgcggcc gcatcccgca ctcgctgcct ggtgccgtgg |
| 541 | ccgcctcctc gggcagcccc ccgggctcgg cgctggcggc agtggcgagc ggcggagacc |
| 601 | tcttcccggg gcagccggtg tccgaactga tcgcgcagct gctgcgcgct gagccctacc |
| 661 | ctgcggcggc cggacgcttc ggcgcagggg gcggcgcggc gggcgcggtg ctgggcatcg |
| 721 | acaacgtgtg cgagctggcg gcgcggctgc tcttcagcac cgtggagtgg gcgcgccacg |
| 781 | cgcccttctt ccccgagctg ccggtggccg accaggtggc gctgctgcgc ctgagctgga |
| 841 | gcgagctctt cgtgctgaac gcggcgcagg cggcgctgcc cctgcacacg gcgccgctac |
| 901 | tggccgccgc cggcctccac gccgcgccta tggccgccga gcgcgccgtg gctttcatgg |
| 961 | accaggtgcg cgccttccag gagcaggtgg acaagctggg ccgcctgcag gtcgactcgg |
| 1021 | ccgagtatgg ctgcctcaag gccatcgcgc tcttcacgcc cgacgcctgt ggcctctcag |
| 1081 | acccggccca cgttgagagc ctgcaggaga aggcgcaggt ggccctcacc gagtatgtgc |
| 1141 | gggcgcagta cccgtcccag ccccagcgct tcgggcgcct gctgctgcgg ctccccgccc |
| 1201 | tgcgcgcggt ccctgcctcc ctcatctccc agctgttctt catgcgcctg gtggggaaga |
| 1261 | cgcccattga gacactgatc agagacatgc tgctgtcggg gagtaccttc aactggccct |
| 1321 | acggctcggg ccagtgacca tgacggggcc acgtgtgctg tggccaggcc tgcagacaga |
| 1381 | cctcaaggga cagggaatgc tgaggcctcg aggggcctcc cggggcccag gactctggct |
| 1441 | tctctcctca gacttctatt ttttaaagac tgtgaaatgt ttgtcttttc tgttttttaa |
| 1501 | atgatcatga aaccaaaaag agactgatca tccaggcctc agcctcatcc tccccaggac |
| 1561 | ccctgtccag gatggagggt ccaatcctag gacagccttg ttcctcagca cccctagcat |
| 1621 | gaacttgtgg gatggtgggg ttggcttccc tggcatgatg gacaaaggcc tggcgtcggc |
| 1681 | cagaggggct gctccagtgg gcaggggtag ctagcgtgtg ccaggcagat cctctggaca |
| 1741 | cgtaacctat gtcagacact acatgatgac tcaaggccaa taataaagac atttcctacc |
| 1801 | tgca |

which corresponds to the following amino acid sequence (S83E mutation underlined):

(SEQ ID NO: 8)
MAMVTGGWGGPGGDTNGVDKAGGYPRAAEDDSASPPGAASDAEPGDEE
RPGLQVDCVVCGDKSSGKHYGVFTCEGCKSFFKREIRRNLSYTCRSNR
DCQIDQHHRNQCQYCRLKKCFRVGMRKEAVQRGRIPHSLPGAVAASSG
SPPGSALAAVASGGDLFPGQPVSELIAQLLRAEPYPAAAGRFGAGGGA
AGAVLGIDNVCELAARLLFSTVEWARHAPFFPELPVADQVALLRLSWS
ELFVLNAAQAALPLHTAPLLAAAGLHAAPMAAERAVAFMDQVRAFQEQ
VDKLGRLQVDSAEYGCLKAIALFTPDACGLSDPAHVESLQEKAQVALT
EYVRAQYPSQPQRFGRLLLRLPALRAVPASLISQLFFMRLVGKTPIET
LIRDMLLSGSTFNWPYGSGQ

Substitution of Ser-83 with alanine resulted in a decreased steady-state DNA-binding (most likely due to a conformational change resulting from the substitution) but in contrast to the NR2F6 wild-type, DNA-binding of this S83A mutant remained principally unchanged between resting and CD3/CD28-activated cells. Yet, and in strict contrast to the NR2F6 wild-type and S83A mutant, replacement of Ser-83 with glutamic acid (as Ser-83 phospho-mimic mutant), abrogated the DNA-binding of NR2F6 in nuclear extracts in resting and stimulated cells. These results suggest that, in part, the DNA binding of NR2F6 might be antagonized by a (p)Ser-83 switch on NR2F6, controlled by SIT (serine/threonine) protein kinases such as PKC family members, known to be rapidly activated after CD3/CD28 ligation (Tan(2003) Biochem. J. 376:545-552; Spitaler (2004) Science 288:2369-2373). Quite similarly, the DNA binding of another nuclear orphan receptor, Nur77, is inhibited by a direct phosphorylation within its DNA-binding domain during survival signalling via Akt/PKB (Masuyama (2001) J. Biol. Chem. 276, 32799-32805).

Protein kinase assay: PKC isotypes dependent phosphorylation was measured by incorporation of ³²P; from γ³²P ATP incubating 200 ng of purified recombinant GST-NR2F6 in vitro. Briefly, 40 mM Tris pH7.5, 40 mM MgCl₂, 0.2 mM DTT, 0.0002% Triton X-100, 0.3 mg ml⁻¹ BSA) containing 1 μM ATP, 2 μCi [³²P-ATP], 1 μM PDBu and 160 μM phosphatidylserine. After 20 min at 30° C., the reaction was stopped by adding stopp solution (10 mM ATP, 5 mM EGTA pH7.5, 0.1% Triton X-100). Incorporated radioactivity was measured by SDS-PAGE and X-ray autoradiography.

GST pull down assay: 1×10⁷ Jurkat T cells were lysed in 1 ml lysis buffer (25 mM MES pH6.5, 5 mM NaF, 1 mM Na₃VO₄, 2 mM EDTA, 150 mM NaCl, 0.5% TritonX-100, 50 μg/ml aprotinin and leupeptin). Lysates were precleared for 30 min at 4° C. Thereafter incubation with purified recombinant GST-NR2F6 fusion proteins for 3 h at 4° C., 5× washing in lysis buffer, SDS-PAGE under reducing conditions on Bis/Tris-buffered gels (Novex, San Diego, Calif.) was performed. Proteins were transferred onto a polyvinyl-difluoridon (PVDF) membrane (Millipore, Bedford, Mass.) by semi-dry blotting (90 mA, 80 min, 4° C.). The primary Abs against PKCO (Cell Signalling) or PKCα (UBI) were diluted in Tris-buffered saline (TBS) containing 0.5% Tween-20 and 5% non-fat dry milk for incubation. Peroxidase-conjugated antibodies (Pierce, Rockford, Ill.) served as secondary reagent (1:5,000). For antigen detection enhanced chemiluminescence was used (Super Signal, Pierce, Rockford, Ill.).

NR2F6 immunoprecipitation: 1×10⁷ Jurkat T cells were lysed in 1 ml lysis buffer (25 mM MES pH6.5, 5 mM NaF, 1 mM Na₃VO₄, 2 mM EDTA, 150 mM NaCl, 0.5% TritonX-100, 50 μg/ml aprotinin and leupeptin). Lysates were precleared for 30 min at 4° C. Immunoprecipitation was performed at 4° C. overnight. Thereafter incubation with Protein G Sepharose (Amersham-Pharmacia, Vienna) for 1 h at 4° C., 5× washing in lysis buffer, SDS-PAGE under reducing conditions on Bis/Tris-buffered gels (Novex, San Diego, Calif.) was performed. Proteins were transferred onto a polyvinyl-difluoridon (PVDF) membrane (Millipore, Bedford, Mass.) by semi-dry blotting (90 mA, 80 min, 4° C.). The (p)S-83 NR2F6 polyclonal antiserum was raised against the 12 amino-acid NH₂-SFFKR-(p)S-IRRNL-COOH phosphopeptide. The primary Abs against NR2F6 was from (Perseus Proteomics; R&D). Peroxidase-conjugated antibodies (Pierce, Rockford, Ill.) served as secondary reagent (1:5,000). For antigen detection enhanced chemiluminescence was used (Super Signal, Pierce, Rockford, Ill.).

Cells and transfections: Jurkat T cells were maintained in RPMI medium supplemented with 10% FCS (Life Technologies, Inc.). Transient transfection of cells was performed by electroporation in a BTX-T820 ElectroSquarePorator (ITC, Biotech, Heidelberg, Germany) apparatus using predetermined optimal conditions: 2×10⁷ cells at 450V/cm and 5 pulses of 99 msec. Optimix medium (Equibio, Kent, UK) was used for studies of promoter reporter gene expression.

Gel mobility shift assay: Jurkat T cells were transfected as described with NR2F6 wild-type or S83A and S83E mutant or GFP inert protein control, subcloned into the pEF-neo expression vector, and were either left unstimulated or were stimulated with solid-phase CD3 and CD28 antibodies for 16 hrs. Subsequently, nuclear extracts were harvested from 1-2×10⁷ cells. Cells were washed in PBS and resuspended in 10 mM HEPES (pH 7.9), 10 mM KCl, 0.1 mM EDTA, 0.1 mM EGTA, 1 mM DTT, and protease inhibitors. Cells were incubated on ice for 15 min. Nonidet P-40 was added to a final concentration of 0.6%, and cells were vortex-mixed vigorously, and the mixture was centrifuged for 5 min. The nuclear pellets were washed twice and resuspended in 20 mM HEPES (pH 7.9), 0.4 M NaCl, 1 mM EDTA, 1 mM EGTA, 1 mM DTT, and protease inhibitors, and the tube was rocked for 30 min at 4° C. After centrifugation for 10 min, the supernatant was collected. Extract proteins (2 μg) were incubated in binding buffer with ³²P-labeled, double-stranded oligonucleotide probes (NF-κB, 5′-GCC ATG GGG GGA TCC CCG AAG TCC-3′ (SEQ ID NO:15); AP-1,5′-CGC TTG ATG ACT CAG CCG GAA-3′ (SEQ ID NO:16); and NF-AT, 5′-GCC CAA AGA GGA AAA TTT GTT TCA TAC AG-3′ (SEQ ID NO:17) In each reaction, 3×10⁵ cpm of labelled probe was used, and band shifts were resolved on 5% polyacrylamide gels. Supershifts were performed using the monoclonal anti-human NR2F6 antibody (Perseus proteomics:R&D Systems).

Immunoblotting of recombinant NR2F6 expression: Untransfected cells or cells transfected with were lysed in ice-cold lysis buffer (5 mM NaP₂P, 5 mM NaF, 5 mM EDTA, 50 mM NaCl, 50 mM Tris (pH 7.3), 2% Nonidet P-40, and 50 μml⁻¹ each aprotinin and leupeptin) and centrifuged at 15,000×g for 15 min at 4° C. Protein lysates were subjected to immunoblotting using Abs against NR2F6 (Perseus Proteomics; R&D).

### Example 3

### NR2F6 is Expressed in Immune Relevant Tissues

Expression of NR2F6 mRNA has been reported to be high in the embryonic brain and in developing liver (Miyajima (1988) Nucleic Acids Res. 16:11057-11074; Warnecke (2005) Genes & Development 19:614-625). Surprisingly, NR2F6 expression was also found in the E14.5 embryonic thymus (FIG. 2H) and its pattern differed from the other COUP-TF family members NR2F1 and NR2F2. NR2F6 was also expressed in the spleen, lymph node, bone marrow (FIG. 21), CD3⁺ T and CD19⁺ B lymphocytes (not shown), indicating a potential functional role of NR2F6 in the immune system. In CD3⁺ lymphocytes, T cell activation was associated with a strong decrease in NR2F6 mRNA levels, suggesting a silencing effect on NR2F6 gene transcription by the T cell receptor (TCR)-mediated signalling pathway (FIG. 2J).

In situ hybridization: Non-radioactive ISH on 20-μm cryostat sections was carried out with digoxygenin-labeled antisense riboprobes. Templates for riboprobes were generated from embryonic NR2F6 cDNA.

RNA transcript analysis: RNA was prepared from cells and tissues indicated in the text with either Trizol (Invitrogen) or the MagAttract direct mRNA M48 kit (Qiagen), first strand cDNA synthesis was performend using oligo(dT) primers (Promega) using the Qiagen Omniscript RT kit according to the instructions of the supplier. Samples were subject to real-time PCR analysis in duplicates on an ABI PRIM 7000 Sequence Detection System (Applied Biosystems) using TaqMan gene expression assays for GAPDH VIC labelled and NR2F6 (Mm01340321-m1) FAM labelled.

### Example 4

### Identification of Over-Reactive Immune Phenotype in NR2F6 Deficient Mice

### Normal Differentiation/Development of T and B Cells in NR2F6 Deficient Mice

To explore the in vivo function of NR2F6 in the adaptive immune system, T and B lymphocyte maturation were compared between NR2F6^{-/-} mice and their wild-type NR2F6^{+/+} controls. In NR2F6^{-/-} mice, CD4⁺/CD8⁺ double positive thymocytes were able to differentiate into normal numbers of CD4⁺ single positive cells, but a reduction of single positive CD8⁺ T cell numbers was observed; see Table 1 as provided herein below:

Thymus

| | NR2F6^{+/+} | NR2F6^{-/-} |
|---|---|---|
| total (×10⁶) | 241.6 ± 9.0 | 206.6 ± 20.1 |
| CD4⁺CD8⁻ | 20.3 ± 4.1 | 17.5 ± 4.4 |
| CD4⁻CD8⁺ | 7.1 ± 0.6 | 3.8 ± 0.8 |
| CD4⁺CD8⁺ | 200.4 ± 8.3 | 175.9 ± 16.3 |

B lymphocyte development in the bone marrow of NR2F6 knockout mice was not significantly different from wild-type controls thereby resulting in normal B cell subsets in young (6-10 weeks-old) knockout mice; see FIG. 8 and Table 2 as provided as herein below

Spleen (6-10 weeks old mice)

| | NR2F6^{+/+} | NR2F6^{-/-} |
|---|---|---|
| total (×10⁶) | 92.6 ± 10.6 | 79.0 ± 8.2 |
| T cells | | |
| CD4 | 16.2 ± 1.1 | 18.8 ± 1.4 |
| CD44^{lo}/CD62L^{hi} | 9.6 ± 1.5 | 12.7 ± 1.7 |
| CD44^{hi}/CD62L^{lo} | 7.4 ± 2.4 | 4.9 ± 0.6 |
| CD8⁺ | 11.3 ± 0.8 | 8.6 ± 0.7 |
| CD44^{lo}/CD62L^{hi} | 7.4 ± 0.8 | 6.4 ± 0.8 |
| CD44^{hi}/CD62L^{lo} | 3.2 ± 1.3 | 1.6 ± 0.4 |
| B cells | | |
| CD19⁺ | 29.1 ± 4.1 | 35.0 ± 4.4 |
| IgM/IgD | 34.1 ± 3.7 | 41.9 ± 5.0 |
| T1 | 2.8 ± 0.4 | 2.7 ± 0.4 |
| MZ | 3.4 ± 1.0 | 6.1 ± 1.8 |

NR2F6^{-/-} thymocytes demonstrated normal susceptibility to apoptosis responses both in vitro (not shown) and during anti-CD3 antibody-mediated depletion of CD4⁺/CD8⁺ double positive thymocytes in vivo (FIG. 9), suggesting normal sensitivity to negative selection signals of immature thymocytes. Consistently, FACS analysis of spleen and lymph nodes of 6-10 weeks old NR2F6^{-/-} mice revealed no gross differences in the distribution of CD3, CD4, CD8, and CD19 positive cells. In spleens, surface expression of CD3, CD4, CD8, CD44, CD62L, ICOS, TCR, Vβ8, CD19, B220, IgM, IgD, CD43, CD5, CD21, CD23, Ger1, Mac1, Thy1 numbers and ratios of T and B cell populations were comparable to those of NR2F6^{+/+} mice (see Table 2 as provided herein above and data not shown) suggesting that NR2F6 is not essential for normal lymphocytes development.

### Reduced Activation Threshold of NR2F6 Deficient Immune Cells

To determine the function of NR2F6 in peripheral T and B cells, we analysed their activation thresholds. NR2F6^{-/-} CD3⁺ T cells were more sensitive towards CD3, CD31CD28 and allogeneic MHC (major histocompatibility complex) stimulation (FIGS. 3A & B). This higher activation response was not due to an upregulation of CD3 receptor on the surface of CD3⁺ T cells as shown by flow cytometry (FIG. 3C). Unlike the CD8⁺ T cells, only the CD4⁺ T subset did show enhanced proliferation responses upon CD3/CD28 stimulation (FIG. 10). NR2F6^{-/-} CD4⁺ T cells produced significantly more IL-2 than NR2F6^{+/+} wild-type T cells after CD3/CD28 stimulation (FIG. 3D), whereas NR2F6 IFNgamma secretion levels of CD8⁺ T cells did not differ significantly between both genotypes (FIG. 3E). In addition, NR2F6^{-/-} B cells displayed enhanced proliferative responses upon IgM plus IL-4 stimulation (FIG. 3F). Of note, the expression of CD44, a key component for cell adhesion and migration was found to be up-regulated in both NR2F6^{-/-} untreated and stimulated T cells, suggesting a higher activation status of NR2F6^{-/-} T cells in vivo (FIG. 11). Gene ablation of NR2F6 resulted in a profound augmentation of IL-2 plasma levels 2 hrs after i.p. injection of super antigen staphylococcal enterotoxin B (SEB), which selectively activates the TCR-Vβ8-positive T cells (FIGS. 3G & H), suggesting a critical role of NR2F6 in signal attenuation of T cell antigen receptor signalling also in vivo.

### Negative Regulation of NF-AT and AP-1 by NR2F6 in CD4⁺ T Cells

In the absence of NR2F6 no differences in the extents or kinetics of membrane-proximal phosphorylation events, as (p)Tyr-783 on PLCγ1, (p)Ser-32 on I-κBα or on the activation loops on (p)ERK1/2 could be observed upon CD3/CD28 stimulation of CD3⁺ T cells (FIG. 4A). However, analysis of nuclear extracts with critical enhancer sequences in EMSA, demonstrated elevated NF-AT and AP-1 DNA binding in CD3⁺ T cells derived from NR2F6-knockout mice (FIG. 4B). Similarly, activated NR2F6^{-/-} CD4⁺ T cells showed a strong augmentation in DNA-binding of both NF-AT and AP-1, when compared to wild-type controls (FIGS. 4C & D). However, AP-1 activation was normal in the NR2F6^{-/-} CD8⁺ T cell subset, compared to wild-type CD8⁺ T cell controls (FIG. 4D). Unlike NF-AT and AP-1, NF-κB activation was not affected by the absence of NR2F6 in neither T cell subset (FIGS. 4B & E). These data indicated that particularly in CD4⁺ T cells, NR2F6 functions selectively as a negative regulator of CD3/CD28-induced NF-AT and AP-1 transactivation responses.

### NR2F6^{-/-} Mice Develop a Late-Onset Immunopathology

NR2F6^{-/-} CD4⁺ (but not CD8⁺) T cell blasts, expanded from concanavalin A/IL-2-treated peripheral CD3⁺ T cells, demonstrated a reduced rate of apoptotsis upon re-stimulation with anti-CD3 antibodies in vitro (FIGS. 12, A & B). NR2F6^{-/-} CD4⁺ T cells were, however, essentially as sensitive as the NR2F6^{+/+} wild-type cells to treatment with a non-related apoptosis trigger, such as the DNA-damaging agent and etoposide (not shown). Of note, in one-year old mice most T cells displayed an effector/memory phenotype; see Table 3 as provided herein below.

Spleen (1 year old mice)

| | NR2F6^{+/+} | NR2F6^{-/-} |
|---|---|---|
| total (×10⁶) | 64.8 ± 6.2 | 131.6 ± 26 |
| T cells | | |
| CD3⁺ | 26.6 ± 7.1 | 45.3 ± 11 |
| CD4⁺ | 13.6 ± 2.7 | 30.1 ± 6.2 |
| CD44^{lo}/CD62L^{hi} | 7.1 ± 0.4 | 11.6 ± 0.9 |
| CD44^{hi}/CD62L^{lo} | 4.8 ± 0.6 | 13.0 ± 5.5 |
| CD8⁺ | 8.7 ± 1.3 | 8.9 ± 2.7 |
| CD44^{lo}/CD62L^{hi} | 3.9 ± 0.6 | 3.6 ± 0.4 |
| CD44^{hi}/CD62L^{lo} | 6.3 ± 0.9 | 8.0 ± 2.0 |
| B cells | | |
| CD19⁺ | 32.6 ± 3.5 | 62.4 ± 15.5 |
| IgM/IgD | 17.5 ± 2.1 | 35.4 ± 9.3 |
| T1 | 3.6 ± 0.8 | 6.5 ± 1.5 |
| T2 | 1,5 ± 0.2 | 2.5 ± 0.3 |
| MZ | 2.8 ± 0.5 | 5.6 ± 1.5 |

NR2F6^{-/-} B cells were also more refractory than wild-type cells to spontaneous apoptosis as well as apoptosis induced by B cell receptor ligation using anti-IgM Fab fragments in vitro (FIGS. 12, C & D).

With increasing age (≧12 months) NR2F6 deficiency led to hyperplasia revealing a lymphocyte homeostasis defect in vivo. Specifically, CD4⁺ T cells and MZ-B cells were significantly increased in older NR2F6^{-/-} mice (see Table 3 as provided herein above). Despite normal lymph nodes, about half of the aged NR2F6^{-/-} mice showed enlarged spleens (FIG. 5A), bearing approximately twice as many lymphocytes (FIG. 5B-E). One-year old NR2F6^{-/-} mice demonstrated signs of autoimmunity, since significantly higher titters of IgG1 (FIG. 5F and FIG. 13) as well as autoantibodies against nuclear antigens (ANA) including double-stranded (ds) DNA (FIG. 5G-I) were detected.

Flow cytometry: Single-cell suspensions were prepared and incubated for 30 min on ice in staining buffer (PBS containing 2% FCS and 0.2% NaN₃) with FITC, PE, APC or biotinylated Ab conjugates. Surface marker expression of thymocytes, splenocytes or lymph node cells were analyzed using a FACS Calibur cytometer (BD Biosciences) and CellQuestPro software according to standard protocols. Abs against murine CD3, CD4, and CD8 were obtained from Caltag Laboratories and CD28, CD69, CD44, CD25, CD62L, ICOS, TCR, Vbeta8, CD19, B220, IgM, IgD, CD43, CD5, CD21, CD23, Ger1, Mac1, Thy1 and CD19 were obtained from BD Pharmingen, FoxP3 from e-Bioscience respectively. Single cell suspensions derived from 2 femurs/mouse were counted in a hemocytometer and stained with cocktails of anti-B220-FITC (clone RA3-6B2), anti-IgM-APC (clone II/41), anti-IgD-FITC (clone 11-26), anti-CD21/CD35-bio (clone 7G6) and anti-CD43-bio (clone R2/60) Abs for 30 min at 4° C. All antibodies were purchased from eBioscience. Streptavidin-RPE (Dako) was used to stain biotinylated mAbs.

Analysis of proliferation responses: Antibody- and alloantigen-induced proliferation was measured by [³H]thymidine incorporation during the last 16 hrs of incubation. Naive mouse CD3⁺ T cells were purified from pooled spleen and lymph nodes with mouse T cell enrichment columns (R&D Systems). CD4⁺ and CD8⁺ T cells were negatively selected by magnetic cell sorting, according to manufacturers instructions (Miltenyi Biotec). For anti-CD3 stimulations, T cells (5×10⁵) in 200 μl proliferation medium (RPMI supplemented with 10% FCS, 2 mM L-glutamine and 50 units ml⁻¹ penicillin/streptomycin) were added in duplicates to plates precoated with anti-CD3 antibody (clone 2C11, 10 μg ml⁻¹). Where indicated soluble anti-CD28 (1 μg ml⁻¹; BD Bioscience) was added. Splenic B cells were purified by depletion of non-B cells on MACS columns (Miltenyi Biotec) with anti-CD43 Abs coupled to magnetic beads (Miltenyi Biotec). The purity of B cells was typically 95%, as determined by staining and flow cytometry. B cells were stimulated with 1.2 or 2.4 μg ml⁻¹ goat anti-mouse IgM F(ab′)₂ (Dianova) in combination with 25 U ml⁻¹ recombinant mouse IL-4 (Roche). Cells were harvested on filters at 50 or 64 hrs after a 16 hrs pulse with [³H]thymidine (1 μCi/well) and incorporation of [³H]thymidine was measured with a Matrix 96 direct β counter system. Results shown are the means±SD of at least three independent experiments.

Analysis of cytokine production: 1 μg SEB (Staphylococcus enterotoxin B) was injected i.p. in parallel to PBS control, animals were sacrificed 2 hrs later and serum was collected and analyzed via BioPlex technology (BioRad). Cytokine production in mouse CD3⁺, CD4⁺ or CD8⁺ T cells after antibody stimulation or antigenic-peptide challenge in spleen cells from mice was assessed via the BioPlex technology (BioRad) according to manufacturer's instructions. Results shown are the means±SD of at least three independent experiments.

Western blot analysis: T cells were stimulated with solid-phase hamster anti-CD3 (clone 145-2C11), with or without hamster anti-CD28 (clone 37.51; BD Biosciences) at 37° C. for various time periods. Cells were lysed in ice-cold lysis buffer (5 mM NaP₂P, 5 mM NaF, 5 mM EDTA, 50 mM NaCl, 50 mM Tris (pH 7.3), 2% Nonidet P-40, and 50 μg ml⁻¹ each aprotinin and leupeptin) and centrifuged at 15,000×g for 15 min at 4° C. Protein lysates were subjected to immunoblotting using Abs against (p)Y-783 PLCγ1 (Cell Signalling), PLCγ1, Fyn (Santa Cruz Biotechnology), (p)S-32 IκBα (Cell signalling); (p)ERK and ERK.

Gel mobility shift assay: Nuclear extracts were harvested from 1-2×10⁷ CD3⁺, CD4⁺ or CD8⁺ T cells that were purified as described herein above. Purified cells were washed in PBS and resuspended in 10 mM HEPES (pH 7.9), 10 mM KCl, 0.1 mM EDTA, 0.1 mM EGTA, 1 mM DTT, and protease inhibitors. Cells were incubated on ice for 15 min. Nonidet P-40 was added to a final concentration of 0.6%, and cells were vortex-mixed vigorously, and the mixture was centrifuged for 5 min. The nuclear pellets were washed twice and resuspended in 20 mM HEPES (pH 7.9), 0.4 M NaCl, 1 mM EDTA, 1 mM EGTA, 1 mM DTT, and protease inhibitors, and the tube was rocked for 30 min at 4° C. After centrifugation for 10 min, the supernatant was collected. Extract proteins (2 μg) were incubated in binding buffer with ³²P-labeled, double-stranded oligonucleotide probes (NF-κB, 5′-GCC ATG GGG GGA TCC CCG AAG TCC-3′ (SEQ ID NO:15); AP-1,5″-CGC TTG ATG ACT CAG CCG GAA-3′ (SEQ ID NO:16); and NF-AT, 5′-GCC CAA AGA GGA AAA TTT GTT TCA TAC AG-3′ (SEQ ID NO:17) (Nushift; Active Motif). For NR2F6 the EMSA Probe set of Panomics for NR2F1 (AY1284P) with the sequence 5′-GTGTCAAAGGTCGTGTCAAAGGTC-3′ (SEQ ID NO:18) which contains the same binding sequence as NR2F6 was used. In each reaction, 3×10⁵ cpm of labelled probe was used, and band shifts were resolved on 5% polyacrylamide gels. Supershifts were performed using the following antibodies NR2F6: monoclonal anti-humanEar2/Nr2F6 antibody (Perseus proteomics:R&D Systems); AP-1 cFos; NF-ATc; NFK-B p50 (all Nushift; Active Motive).

Apoptosis detection: Freshly isolated thymocytes from 6-8 week old mice were plated in 96 well plates at a density of 2.5×10⁵ cells/well in a total volume of 200 μl (RPMI/10% FCS/2 mM L-Glutamin/50 u ml⁻¹ Pen/Strep). Apoptosis induction was performed by addition of either Con A (10 μg ml¹), phorbol 12,13-dibutyrate (PDBu; 1 μg ml⁻¹), ionomycin (1 μml⁴), camptothecin (1 μM), dexamethasone (10⁻⁶ M), or staurosporin (100 nM). Percentage of viable cells was determined by propidium iodide staining at time points between 10 and 40 hrs after apoptosis induction using a FACScan cytometer (BD Biosciences) and CellQuestPro software. Total splenocytes were used to generate activated T cell blasts using Con A (2 μml⁻¹) for 48 hours, followed by IL-2 stimulation (100 u ml⁻¹) for additional 72 hrs. (IMEM/10% FCS/2 mM L-Glutamin/50 u ml⁻¹ Pen/Strep) After 5 days, activated T cell blasts were washed twice in medium, viable cells were enriched by Lympholyte™ (Cedarlane) gradient centrifugation (viability >90%), and incubated in IMEM medium (10% FCS/2 mM L-Glutamin/50 u ml⁻¹ Pen/Strep). Apoptosis sensitivity was challenged by different concentrations of anti-CD3 cross-linking Abs (clone 2C11) or cross-linked recombinant FasL (FasL 100 ng ml⁻¹ & enhancer for FasL at 1 μg ml⁻¹) to induce activation-induced cell death. 8 hrs after apoptosis induction cells were harvested and stained with annexin V-FITC (Molecular Probes), anti CD4-PE and anti CD8-APC (Caltag). The percentage of apoptotic cells in each T cell subset was determined by FACS analysis using FACSCalibur (BD) and CellQuestPro software. Spenic B cells from wt and NR2F6^{-/-} mice were purified in a FACSvantage sorter by negative cell sorting using following monoclonal antibodies: MI/70, anti-Mac-1 (ebioscience); Ter119, antierythroid cell surfacemarker (BD); T24.31.2, anti-Thy-1. B cells were cultured at an initial concentration of 5×10⁵ cells ml⁻¹ in RPMI medium 1640 supplemented with 4.5 g l⁻¹ glucose (Cambrex), 50 μM β-mercaptoethanol (Merck), 10 mM Hepes (Sigma-Aldrich), 2 mM L-Glutamine (Cambrex), 1 mM Sodium pyruvate (PAA), 100 mM non essential amino acids (Gibco), 100 μg ml⁻¹ penicillin and 100 u ml⁻¹ streptomycin (Cambrex), 500 μg ml⁻¹ gentamycine (Gibco) and 10% fetal bovine serum (PAA). For the induction of cell death anti-IgM F(ab) fragments (Jackson Immuno Research) were used. The percentages of viable cells in culture were determined by staining cell suspensions with 2 μg ml⁻¹ PI plus annexin V-FITC (Molecular Probes).

Determination of serum immunoglobulin (Ig) concentrations: The concentration of different Ig subclasses in mouse sera were analyzed using isotype-specific antibodies using the following ELISA Quantification kits IgGa (Bethyl: E90-107), IgG1 (Bethyl: E90-105), IgG (Bethyl: E90131), IgE (Bethyl: E90-115), IgM (Bethyl: E90-101) following the manufacturer's instructions.

Detection of autoantibodies in mouse serum on rat liver sections and with ELISA: Slides precoated with rat liver sections were used to evaluate the presence of ANA in dilutions obtained from mouse blood serum using a Alexa Fluor 488 goat anti-mouse IgG1 antibody (Molecular Probes). Measurement of autoantibodies against double-stranded DNA, and single-stranded DNA were performed by ELISA (αDiagnostics), ANA ELISA No. 5200 and Anti-dsDNA No. 5100) and performed according to manufacturer's instructions. All samples were assayed in duplicate and results expressed as an absorbance value at 450 nM.

### Example 5

### NR2F6-Deficient Mice are More Susceptible to Antigen-Induced Autoimmunity

To gain more insight into NR2F6 immune function in vivo, we employed the experimental autoimmune encephalomyelitis (EAE) model, a multiple sclerosis-like autoimmune disease induced by immunizing 6-10 week old female mice with the myelin component MOG₃₅₋₅₅. Consistent with our hypothesis that NR2F6 may be critical for normal immune regulation and the preclusion of autoimmunity, NR2F6^{-/-} mice were found to be significantly more susceptible to EAE induction. When the progressive paralysis from tail to the head was scored, NR2F6^{-/-} mice demonstrated both a faster onset and an overall higher clinical score (FIG. 6A). The pro-inflammatory T helper cell subpopulation, termed Th17 has been known to play the critical role in the mouse EAE disease progression. In fact, IL-17 production rates from the CD4⁺ T cell subset has been specifically connected with the “decision making” between immunological tolerance and autoimmunity (Harrington (2005) Nature immunology 6, 1123-1132; Harrington (2006) Curr Opin Immunol 18, 349-356; McKenzie (2006) Trends Immunol 27, 17-23; Bettelli (2007) Nature immunology 8, 345-350). Consistently, accelerated disease in the NR2F6^{-/-} mice was associated with a significant increase of IFNgamma and, particularly, IL-17 cytokine responses in MOG₃₅₋₅₅ antigen-dependent recall assays ex vivo (FIGS. 6B & C), confirming that Th17 cell functions were hyper-reactive in NR2F6 deficient mice. Numbers and ratios of T and B cell populations in EAE-diseased mice did not differ between genotypes including the numbers and ratios of both FOXP3⁺ Treg and Th17 cell lineages (FIG. 14). Similarly to CD3/CD28 stimulation of naïve CD3⁺ T cells (FIG. 2H), application of the MOG₃₅₋₅₅ antigenic peptide to memory CD3⁺ T cells isolated from MOG₃₅₋₅₅-immunized mice leads to a decrease in NR2F6 mRNA expression level (FIG. 6D). Collectively, the in vivo data physiologically established NR2F6 as a transcriptional repressor of cytokine transcription. NR2F6 function counter-balanced T cell signalling thresholds, thereby preventing autoimmunity.

Induction of EAE: MOG₃₅₋₅₅ peptide was synthesized by NeoSystems, France. Female 8-12 week old mice were immunized by one injection of 200 μg MOG₃₅₋₅₅ Peptide in CFA, supplemented with 5 μg ml⁻¹ Mycobacterium tuberculosis H37 Ra (Difco Laboratories), 1:1 in PBS emulsified in a total of 200 μl. In addition, 200 ng of pertussis toxin (Sigma.Aldrich) dissolved in 200 μl PBS was injected 24 and 72 hrs later i.v. (tail vein). Mice were monitored daily for clinical signs of EAE and graded on a scale of increasing severity from 0 to 4 by two independent investigators. Following scale was used: 0 no symptoms; 0.5 distal weak or spastic tail; 1.0 complete limp tail; 1.5 limp tail and hind limb weakness (feet slip through cage grill); 2.0 unilateral partial hindlimb paralysis; 2.5 bilateral partial hindlimb paralysis; 3.0 complete bilateral hindlimb paralysis; 3.5 complete hindlimb and unilateral partial forelimb paralysis; 4.0 death or moribund (Salek-Ardakani (2005) J Immunol 175, 7635-7641).

Antigen recall assay: Splenocyte cell suspension and serum were isolated from MOG₃₅₋₅₅ peptide immunized wt and NR2F6^{-/-} mice, on day 21 along with PBS treated wt and mutant mice. Splenocytes from individual mice, depleted of RBC with lysing buffer (R&D), were plated in duplicates (5×10⁵/well) in 200 μl proliferation medium (RPMI supplemented with 10% FCS, 2 mM L-glutamine and 50 U ml⁻¹ penicillin/streptomycin) containing either 0, 1, 10 or 100 μg ml⁻¹ MOG₃₅₋₅₅ Peptide and cultured at 37° C. in 5% CO₂. Cytokine production in cells after MOG₃₅₋₅₅ peptide stimulation was analyzed by removing 60 μl of cell culture supernatant/well after 60 hrs of culture and stored at −80° C. Cytokines were analyzed with BioPlex multianalyte technology (BioRad) according to the manufacturer's instructions.

Flow cytometry: Single-cell suspensions were prepared and incubated for 30 min on ice in staining buffer (PBS containing 2% FCS and 0.2% NaN₃) with FITC, PE, APC or biotinylatedAb conjugates. Surface marker expression of thymocytes, splenocytes or lymph node cells were analyzed using a FACS Calibur cytometer (BD Biosciences) and CellQuestPro software according to standard protocols. Abs against murine CD4, and CD8 were obtained from Caltag Laboratories and CD44, CD62L, CD19, IgM, and CD19 were obtained from BD Pharmingen, FoxP3 from e-Bioscience respectively. Single cell suspensions derived from 2 femurs/mouse were counted in a hemocytometer and stained with cocktails of anti-IgM-APC (clone II/41) and anti-IgD-FITC (clone 11-26) Abs for 30 min at 4° C. All antibodies were purchased from eBioscience except the IL-23R antibody which was from R&D.

RNA transcript analysis: RNA was prepared from cells with the MagAttract direct mRNA M48 kit (Qiagen), first strand cDNA synthesis was performend using oligo(dT) primers (Promega) using the Qiagen Omniscript RT kit according to the instructions of the supplier. Samples were subject to real-time PCR analysis in duplicates on an ABI PRIM 7000 Sequence Detection System (Applied Biosystems) using TaqMan gene expression assays for GAPDH VIC labelled and NR2F6 (Mm01340321-ml) FAM labelled.

### Example 6

### NR2F6-ER Fusion Protein Allows Suppression of NF-AT/AP-1 Activation

When constructing and employing a conditionally activated NR2F6 estrogen-receptor ligand binding domain mutant (ERₘᵤₜ-LBD) fusion protein, termed “NR2F6-ER” (FIG. 15A), the CD3/CD28-induced the NF-AT/AP-1 composite element-dependent reporter was repressed by recombinant NR2F6-ER in transiently transfected Jurkat T cells. Tamoxifen (OHT) as the agonist of ERₘᵤₜ-LBD within the recombinant NR2F6-ER fusion mutant selectively enhanced transrepression of CD3/CD28-stimulation induced NF-AT-promoter reporter luciferase activation in Jurkat T cells. Similarly, phosphorylation status of Ser-83 affected NR2F6 cellular function, since its substitution with glutamic acid (but not with alanine) reduced NR2F6 repressional activity on NF-AT/AP-1-dependent promoter reporter transcription; see FIG. 15C. This teaches that recombinant NR2F6 blocked CD3/CD28-induced transcriptional activation of the NF-AT/AP-1 in NR2F6-ER expressing T cells. This repressor activity was abolished by the S83E mutation on NR2F6, the pseudo-phosphorylation mimic of residue 83, but not by the S83A mutation; see FIG. 15C. Results with recombinant NR2F6 in transfected human Jurkat T cells thus independently validated the antagonism between NF-AT/AP-1 and NR2F6 observed in our knockout mouse studies as described herein above. The equal expression levels of the recombinant NR2F6 in nuclear fractions were confirmed by immunoblotting (FIG. 15B). Accordingly, the gain-of-function fusion mutant NR2F6-ER, as proof of concept, indicates that a NR2F6 agonist (similar to OHT as agonist of the recombinant NR2F6-ER mutant) will induce immunosuppression.

Jurkat T cells were maintained in RPMI medium supplemented with 10% FCS (Life Technologies, Inc.). Transient transfection of cells was performed by electroporation in a BTX-T820 ElectroSquarePorator (ITC, Biotech, Heidelberg, Germany) apparatus using predetermined optimal conditions: 2×10⁷ cells at 450V/cm and 5 pulses of 99 msec. Optimix medium (Equibio, Kent, UK) was used for studies of promoter reporter gene expression. Reporter gene expression was measured in co-transfection assays using 15 μg of the NR2F6 wild-type or mutant expression vectors and 15 μg of the AP-1/NF-AT promoter firefly luciferase reporter (RLU1). For normalization 0.3 μg of the renilla luciferase reporter vector pTK-Renilla-Luc (Promega, Medison, Wi) (RLU2) has been used. After 24 hrs cells were stimulated with tamoxifen (OHT) or solid phase CD3 and CD28 antibodies for 16 hrs or left unstimulated, as indicated.

The coding sequence of the NR2F6 wild-type ER-LBD fusion mutant (NR2F6-ER) as used herein corresponds to the following nucleic acid sequence (start codon in bold, linker sequence double underlined, ERₘᵤₜ-LBD coding sequence in italics):

| (SEQ ID NO: 9) | |
|---|---|
| 123 | atggccat ggtgaccggc ggctggggcg gccccggcgg cgacacgaac ggcgtggaca |
| 181 | aggcgggcgg ctacccgcgc gcggccgagg acgactcggc ctcgcccccc ggtgccgcca |
| 241 | gcgacgccga gccgggcgac gaggagcggc cggggctgca ggtggactgc gtggtgtgcg |
| 301 | gggacaagtc gagcggcaag cattacggtg tcttcacctg cgagggctgc aagagctttt |
| 361 | tcaagcgaag catccgccgc aacctcagct acacctgccg gtccaaccgt gactgccaga |
| 421 | tcgaccagca ccaccggaac cagtgccagt actgccgtct caagaagtgc ttccgggtgg |
| 481 | gcatgaggaa ggaggcggtg cagcgcggcc gcatcccgca ctcgctgcct ggtgccgtgg |
| 541 | ccgcctcctc gggcagcccc ccgggctcgg cgctggcggc agtggcgagc ggcggagacc |
| 601 | tcttcccggg gcagccggtg tccgaactga tcgcgcagct gctgcgcgct gagccctacc |
| 661 | ctgcggcggc cggacgcttc ggcgcagggg gcggcgcggc gggcgcggtg ctgggcatcg |
| 721 | acaacgtgtg cgagctggcg gcg |
| | tgg gat cca |
| 1022 | cgaaatgaa atgggtgctt caggagacat gagggctgcc aacctttggc caagccctct |
| 1081 | tgtgattaag cacactaaga agaatagccc tgccttgtcc ttgacagctg accagatggt |
| 1141 | cagtgccttg ttggatgctg aaccgcccat gatctattct gaatatgatc cttctagacc |
| 1201 | cttcagtgaa gcctcaatga tgggcttatt gaccaaccta gcagataggg agctggttca |
| 1261 | tatgatcaac tgggcaaaga gagtgccagg ctttggggac ttgaatctcc atgatcaggt |
| 1321 | ccaccttctc gagtgtgcct ggctggagat tctgatgatt ggtctcgtct ggcgctccat |
| 1381 | ggaacacccg gggaagctcc tgtttgctcc taacttgctc ctggacagga atcaaggtaa |
| 1441 | atgtgtggaa ggcatggtgg agatctttga catgttgctg gctacgtcaa gtcggttccg |
| 1501 | catgatgaac ctgcagggag aagagtttgt gtgcctcaaa tccatcattt tgcttaattc |
| 1561 | cggagtgtac acgtttctgt ccagcacctt gaagtctctg gaagagaagg accacatcca |
| 1621 | ccgtgtcctg gacaagatca cagacacttt gatccacctg atggccaaag ctggcctgac |
| 1681 | tctgcagcag cagcatcgcc gcctagctca gctccttctc attctttccc atatccggca |
| 1741 | catgagtaac aaaggcatgg agcatctcta caacatgaaa tgcaagaacg ttgtgcccct |
| 1801 | ctatgacctg ctcctggaga tgttggatgc ccaccgcctt catgccccag ccagtcgcat |
| 1861 | gggagtgccc ccagaggagc ccagccagac ccagctggcc accaccagct ccacttcagc |
| 1921 | acattcctta caaacctact acataccccc ggaagcagag ggcttcccca acacgatctg |
| 1981 | a |

which corresponds to the following amino acid sequence (linker amino acids double underlined, ERₘᵤₜ-LBD amino acid sequence in italics):

(SEQ ID NO: 10)
MAMVTGGWGGPGGDTNGVDKAGGYPRAAEDDSASPPGAASDAEPGDEE
RPGLQVDCVVCGDKSSGKHYGVFTCEGCKSFFKRSIRRNLSYTCRSNR
DCQIDQHHRNQCQYCRLKKCFRVGMRKEAVQRGRIPHSLPGAVAASSG
SPPGSALAAVASGGDLFPGQPVSELIAQLLRAEPYPAAAGRFGAGGGA
AGAVLGIDNVCELAWDPRNEMGASGDMRAANLWPSPLVIKHTKKNSPA
LSLTADQMVSALLDAEPPMIYSEYDPSRPFSEASMMGLLTNLADRELV
HMINWAKRVPGFGDLNLHDQVHLLECAWLEILMIGLVWRSMEHPGKLL
FAPNLLLDRNQGKCVEGMVEIFDMLLATSSRFRMMNLQGEEFVCLKSI
ILLNSGVYTFLSSTLKSLEEKDHIHRVLDKITDTLIHLMAKAGLTLQQ
QHRRLAQLLLILSHIRHMSNKGMEHLYNMKCKNVVPLYDLLLEMLDAH
RLHAPASRMGVPPEEPSQTQLATTSSTSAHSLQTYYIPPEAEGFPNTI

The coding sequence of the S83A mutated NR2F6 ER-LBD fusion mutant (S83A-NR2F6-ER) as used herein corresponds to the following nucleic acid sequence (start codon in bold, mutated nucleic acids leading to S83A mutation are underlined, linker sequence double underlined, ERₘᵤₜ-LBD coding sequence in italics):

| (SEQ ID NO: 11) | |
|---|---|
| 123 | atggccat ggtgaccggc ggctggggcg gccccggcgg cgacacgaac ggcgtggaca |
| 181 | aggcgggcgg ctacccgcgc gcggccgagg acgactcggc ctcgcccccc ggtgccgcca |
| 241 | gcgacgccga gccgggcgac gaggagcggc cggggctgca ggtggactgc gtggtgtgcg |
| 301 | gggacaagtc gagcggcaag cattacggtg tcttcacctg cgagggctgc aagagctttt |
| 361 | tcaagcgagc catccgccgc aacctcagct acacctgccg gtccaaccgt gactgccaga |
| 421 | tcgaccagca ccaccggaac cagtgccagt actgccgtct caagaagtgc ttccgggtgg |
| 481 | gcatgaggaa ggaggcggtg cagcgcggcc gcatcccgca ctcgctgcct ggtgccgtgg |
| 541 | ccgcctcctc gggcagcccc ccgggctcgg cgctggcggc agtggcgagc ggcggagacc |
| 601 | tcttcccggg gcagccggtg tccgaactga tcgcgcagct gctgcgcgct gagccctacc |
| 661 | ctgcggcggc cggacgcttc ggcgcagggg gcggcgcggc gggcgcggtg ctgggcatcg |
| 721 | acaacgtgtg cgagctggcg gcg |
| | tgg cat cca |
| 1022 | cgaaatgaa atgggtgctt caggagacat gagggctgcc aacctttggc caagccctct |
| 1081 | tgtgattaag cacactaaga agaatagccc tgccttgtcc ttgacagctg accagatggt |
| 1141 | cagtgccttg ttggatgctg aaccgcccat gatctattct gaatatgatc cttctagacc |
| 1201 | cttcagtgaa gcctcaatga tgggcttatt gaccaaccta gcagataggg agctggttca |
| 1261 | tatgatcaac tgggcaaaga gagtgccagg ctttggggac ttgaatctcc atgatcaggt |
| 1321 | ccaccttctc gagtgtgcct ggctggagat tctgatgatt ggtctcgtct ggcgctccat |
| 1381 | ggaacacccg gggaagctcc tgtttgctcc taacttgctc ctggacagga atcaaggtaa |
| 1441 | atgtgtggaa ggcatggtgg agatctttga catgttgctg gctacgtcaa gtcggttccg |
| 1501 | catgatgaac ctgcagggag aagagctttt gtgcctcaaa tccatcattt tgcttaattc |
| 1561 | cggagtgtac acgtttctgt ccagcacctt gaagtctctg gaagagaagg accacatcca |
| 1621 | ccgtgtcctg gacaagatca cagacacttt gatccacctg atggccaaag ctggcctgac |
| 1681 | tctgcagcag cagcatcgcc gcctagctca gctccttctc attctttccc atatccggca |
| 1741 | catgagtaac aaaggcatgg agcatctcta caacatgaaa tgcaagaacg ttgtgcccct |
| 1801 | ctatgacctg ctcctggaga tgttggatgc ccaccgcctt catgccccag ccagtcgcat |
| 1861 | gggagtgccc ccagaggagc ccagccagac ccagctggcc accaccagct ccacttcagc |
| 1921 | acattcctta caaacctact acataccccc ggaagcagag ggcttcccca acacgatctg |
| 1981 | a |

which corresponds to the following amino acid sequence (S83A mutation underlined, linker amino acids double underlined, ERₘᵤₜ-LBD amino acid sequence in italics):

(SEQ ID NO: 12)
MAMVTGGWGGPGGDTNGVDKAGGYPRAAEDDSASPPGAASDAEPGDEE
RPGLQVDCVVCGDKSSGKHYGVFTCEGCKSFFKRAIRRNLSYTCRSNR
DCQIDQHHRNQCQYCRLKKCFRVGMRKEAVQRGRIPHSLPGAVAASSG
SPPGSALAAVASGGDLFPGQPVSELIAQLLRAEPYPAAAGRFGAGGGA
AGAVLGIDNVCELAWDPRNEMGASGDMRAANLWPSPLVIKHTKKN
SPALSLTADQMVSALLDAEPPMIYSEYDPSRPFSEASMMGLLTNLADR
ELVHMINWAKRVPGFGDLNLHDQVHLLECAWLEILMIGLVWRSMEHPG
KLLFAPNLLLDRNQGKCVEGMVEIFDMLLATSSRFRMMNLQGEEFVCL
KSIILLNSGVYTFLSSTLKSLEEKDHIHRVLDKITDTLIHLMAKAGLT
LQQQHRRLAQLLLILSHIRHMSNKGMEHLYNMKCKNVVPLYDLLLEML
DAHRLHAPASRMGVPPEEPSQTQLATTSSTSAHSLQTYYIPPEAEGFP
NTI

The coding sequence of the S83E mutated NR2F6 ER-LBD fusion mutant (S83E-NR2F6-ER) as used herein corresponds to the following nucleic acid sequence (start codon in bold, mutated nucleic acids leading to S83E mutation are underlined, linker sequence double underlined, ERₘᵤₜ-LBD coding sequence in italics):

| (SEQ ID NO: 13) | |
|---|---|
| 123 | atggccat ggtgaccggc ggctggggcg gccccggcgg cgacacgaac ggcgtggaca |
| 181 | aggcgggcgg ctacccgcgc gcggccgagg acgactcggc ctcgcccccc ggtgccgcca |
| 241 | gcgacgccga gccgggcgac gaggagcggc cggggctgca ggtggactgc gtggtgtgcg |
| 301 | gggacaagtc gagcggcaag cattacggtg tcttcacctg cgagggctgc aagagctttt |
| 361 | tcaagcgaga gatccgccgc aacctcagct acacctgccg gtccaaccgt gactgccaga |
| 421 | tcgaccagca ccaccggaac cagtgccagt actgccgtct caagaagtgc ttccgggtgg |
| 481 | gcatgaggaa ggaggcggtg cagcgcggcc gcatcccgca ctcgctgcct ggtgccgtgg |
| 541 | ccgcctcctc gggcagcccc ccgggctcgg cgctggcggc agtggcgagc ggcggagacc |
| 601 | tcttcccggg gcagccggtg tccgaactga tcgcgcagct gctgcgcgct gagccctacc |
| 661 | ctgcggcggc cggacgcttc ggcgcagggg gcggcgcggc gggcgcggtg ctgggcatcg |
| 721 | acaacgtgtg cgagctggcg gcg |
| | tgg gat cca |
| 1022 | cgaaatgaa atgggtgctt caggagacat gagggctgcc aacctttggc caagccctct |
| 1081 | tgtgattaag cacactaaga agaatagccc tgccttgtcc ttgacagctg accagatggt |
| 1141 | cagtgccttg ttggatgctg aaccgcccat gatctattct gaatatgatc cttctagacc |
| 1201 | cttcagtgaa gcctcaatga tgggcttatt gaccaaccta gcagataggg agctggttca |
| 1261 | tatgatcaac tgggcaaaga gagtgccagg ctttggggac ttgaatctcc atgatcaggt |
| 1321 | ccaccttctc gagtgtgcct ggctggagat tctgatgatt ggtctcgtct ggcgctccat |
| 1381 | ggaacacccg gggaagctcc tgtttgctcc taacttgctc ctggacagga atcaaggtaa |
| 1441 | atgtgtggaa ggcatggtgg agatctttga catgttgctg gctacgtcaa gtcggttccg |
| 1501 | catgagtaac ctgcagggag aagagtttgt gtgcctcaaa tccatcattt tgcttaattc |
| 1561 | cggagtgtac acgtttctgt ccagcacctt gaagtctctg gaagagaagg accacatcca |
| 1621 | ccgtgtcctg gacaagatca cagacacttt gatccacctg atggccaaag ctggcctgac |
| 1681 | tctgcagcag cagcatcgcc gcctagctca gctccttctc attctttccc atatccggca |
| 1741 | catgagtaac aaaggcatgg agcatctcta caacatgaaa tgcaagaacg ttgtgcccct |
| 1801 | ctatgacctg ctcctggaga tgttggatgc ccaccgcctt catgccccag ccagtcgcat |
| 1861 | gggagtgccc ccagaggagc ccagccagac ccagctggcc accaccagct ccacttcagc |
| 1921 | acattcctta caaacctact acataccccc ggaagcagag ggcttcccca acacgatctg |
| 1981 | a |

which corresponds to the following amino acid sequence (S83E mutation underlined, linker amino acids double underlined, ERₘᵤₜ-LBD amino acid sequence in italics):

(SEQ ID NO: 14)
MAMVTGGWGGPGGDTNGVDKAGGYPRAAEDDSASPPGAASDAEPGDEE
RPGLQVDCVVCGDKSSGKHYGVFTCEGCKSFFKREIRRNLSYTCRSNR
DCQIDQHHRNQCQYCRLKKCFRVGMRKEAVQRGRIPHSLPGAVAASSG
SPPGSALAAVASGGDLFPGQPVSELIAQLLRAEPYPAAAGRFGAGGGA
AGAVLGIDNVCELAWDPRNEMGASGDMRAANLWPSPLVIKHTKKN
SPALSLTADQMVSALLDAEPPMIYSEYDPSRPFSEASMMGLLTNLADR
ELVHMINWAKRVPGFGDLNLHDQVHLLECAWLEILMIGLVWRSMEHPG
KLLFAPNLLLDRNQGKCVEGMVEIFDMLLATSSRFRMMNLQGEEFVCL
KSIILLNSGVYTFLSSTLKSLEEKDHIHRVLDKITDTLIHLMAKAGLT
LQQQHRRLAQLLLILSHIRHMSNKGMEHLYNMKCKNVVPLYDLLLEML
DAHRLHAPASRMGVPPEEPSQTQLATTSSTSAHSLQTYYIPPEAEGFP
NTI

### Example 7

### Transfection with NR2F6-Specific siRNAs and NR2F6 Overexpression

siRNA Transfection

CD4+ T cells were negatively selected by magnetic cell sorting (Miltenyi Biotec). T cell populations were typically 95% CD4+, as determined by staining and flow cytometry. Cells were resuspended in solution from Nucleofactor mouse T cell kit and program X-01 following the Amaxa guidelines for cell-line transfection. In brief, up to 1×10⁷ cells were mixed with 1.5 μM of synthetic ON-TARGETp1us siRNA predesigned by Dharmacon (i.e., order code 045088) as pool (see SEQ ID NOs: 19 to 26) or ON-TARGETplussiCONTROL nontargeting pool were nucleofected with the Amaxy Nucleofactor apparatus and immediately transferred into 37° C. prewarmed culture medium and cultured for a minimum of 24 hours before experimental analysis; see also FIG. 16.

### NR2F6 Overexpression

### Overexpression of NR2F6 Protein Diminished Activation Responses

5-fold NR2F6 recombinant overexpression in primary mouse CD4+ T cells significantly suppresses IL-2 secretion responses from CD3/CD28 stimulated cells (see FIG. 21). Similarly, both transfected wild-type NR2F6 and recombinant NR2F6-ER fusion mutant (once activated by OHT) induced repression of CD3 plus CD28-induced activation and reporter luciferase gene transcription of the NF-AT:AP-1 and IL17A promoter reporter in Jurkat T cells (see FIGS. 15 & 29). This strictly depended on DNA-binding activity of NR2F6, since this repressor activity was abolished by mutation in NR2F6 DNA-binding domain, i.e. S83E and C112S, respectively. The nucleic acid sequence and the amino acid of the NR2F6 mutant termed C112S are depicted in SEQ ID NOs: 30 and 31, respectively. This data from NR2F6 overexpression systems are complementary to data from NR2F6-deficient T cells, thus together demonstrating that NR2F6 is a nuclear attenuator that directly interferes with DNA-binding of NF-AT/AP-1 and, subsequently, transcriptional activity of NF-AT/AP-1-dependent, IL-17 gene expression.

RNA transcript analysis: RNA was prepared from cells and tissues indicated in the text with MagAttract direct mRNA M48 kit (Qiagen), first strand cDNA synthesis was performed using oligo(dT) primers (Promega) using the Qiagen Omniscript RT kit according to the instructions of the supplier. Samples were subject to real-time PCR analysis in duplicates on an ABI PRIM 7000 Sequence Detection System (Applied Biosystems) using TaqMan gene expression assays for GAPDH VIC labelled and NR2F6 (Mm01340321-m1) FAM labelled. NR2F6 mRNA expression was normalized based on GAPDH expression.

Analysis of cytokine production: Cytokine production of mouse CD4⁺ T cells after Th17 polarizing stimulation was assessed via the BioPlex technology (BioRad) according to manufacturer's instructions.

### Example 8

### Expression of NR2F6 mRNA

Expression of NR2F6 mRNA has been reported to be high in the embryonic brain and in developing liver (Miyajima (1988) Nucleic Acids Res. 16:11057-11074; Warnecke (2005) Genes & Development 19:614-625). NR2F6 expression was also found in the embryonic thymus, in the spleen, lymph node, bone marrow; see FIG. 24A-E. Furthermore and surprisingly, NR2F6 mRNA demonstrated a selectively high expression in immune disease related Th2 and Th17 CD4+ T cells, but not Th0, Th1 and iTreg CD4+ T cells, indicating a potential functional role of NR2F6 in these particular CD4+ T cell subsets of the immune system.

### Th0, Th1, Th2 and Th17 In Vitro Differentiation

For T cell differentiation naïve CD4⁺ cells were isolated via the CD4⁺ CD62L⁺ T Cell Isolation Kit II (Miltenyi Biotec). Polarization of T cells into Th0, Th1, Th2 or Th17 cells was performed by solid-phase anti-CD3 (5 μg/ml) and soluble anti-CD28 (1 μg/ml) in the absence (for neutral differentiation) or presence of polarizing cytokines (Th0: IL-2 (30 ng/ml); Th1: m IL-12 (10 n g/ml), ° IL-4 (5 μg/ml); Th2: IL-4 (10 ng/ml), αIL-12 (5 μg/ml) and αIFN-γ (5 μg/ml); Th17: IL-23 (10 ng/ml), TGF-β (5 ng/ml), IL-6 (20 ng/ml), αIL-4 (2 μg/ml), αIFN-γ (2 μg/ml) as described (Yang et al., 2008). Supernatant was collected on day 4 or 5 and analyzed via BioPlex multi-analyte technology (BioRad). For FACS analysis cells were washed and restimulated with phorbol 12-myristate 13-acetate (PMA) and ionomycin in the presence of Golgi-plug for 5 h, after which IL-17 producing cells were analyzed via intracellular staining.

RNA transcript analysis: RNA was prepared from cells and tissues indicated in the text with either Trizol (Invitrogen) or the MagAttract direct mRNA M48 kit (Qiagen), first strand cDNA synthesis was performed using oligo(dT) primers (Promega) using the Qiagen Omniscript RT kit according to the instructions of the supplier. Samples were subject to real-time PCR analysis in duplicates on an ABI PRIM 7000 Sequence Detection System (Applied Biosystems) using TaqMan gene expression assays for GAPDH VIC labelled and NR2F6 (Mm01340321-ml) FAM labelled. NR2F6 mRNA expression in Th0, Th1, Th2, Th17 and iTreg differentiated CD4⁺ T cells, data were normalized based on GAPDH expression. Control qRT PCRs are shown in order to validate the proper differentiation of the naïve CD4⁺ T cells into the distinct T helper subsets as Th1 (IFNgamma), the Th2 (IL-4), the Th17 (IL-17) and the iTreg (Foxp3), as indicated.

### Example 9

### Target Synexpression Groups of NR2F6 in Th17 T Cells

Affymetrics micro array expression analysis of Th17 differentiated CD4⁺ T cells, directly comparing wild type and NR2F6 knockout T cells (FIG. 25).

RNA transcript analysis: RNA was prepared from Th17 cells with the MagAttract direct mRNA M48 kit (Qiagen), and Affymetrics analysis was performed with the GeneChip® Mouse Exon 1.0 ST Array that employs approximately four probes per exon and roughly 40 probes per gene. Relative fold induction of Nr2f6−/− T cells is shown in comparison to wild-type controls. Logarithm calculated differences: 1 means two fold induction

### Example 10

### NR2F6 is Required for Efficient Retinoic Acid (RA)-Mediated Immunosuppression of Th17 Cells

Retinoic acid (RA), as a Vitamin A metabolite, is established to form ligands with retinoic acid-related nuclear receptors (RAR) that play pleiotropic roles in various biological processes. RA is known to be as a key modulator of TGF-beta-driven immunesuppression, capable of suppressing the differentiation of Th17 cells and conversely promoting the generation of Foxp3+ iTreg cells. Thus NR2F6 and its functional ligands, once defined, play a key role in balancing pathogenic Th17 versus regulatory iTreg numbers and/or functions.

Nr2f6^{-/-} Th17 T cells have a significantly altered sensitivity towards RA treatment in IL-17 activation responses, when compared to wild type Th17 cells. Consistently, RA abrogates NFAT DNA binding in wild type but not in NR2F6^{-/-} Th17 cells; see FIG. 26. Thus a NR2F6 antagonist will induce (partial) resistance to RA while a NR2F6 agonist will augment RA -mediated immunosuppression. Surprisingly, thus any modulation of this TGF-beta/RA/RAR regulatory module via NR2F6 ligands represents an innovative way to control and modulate a functional immune system.

Analysis of cytokine production: Cytokine production of mouse CD4⁺ T cells after Th17 polarizing stimulation was assessed via the BioPlex technology (BioRad) according to manufacturer's instructions.

Gel mobility shift assay: Nuclear extracts were harvested from 1-2×10⁷ T cells that were purified as described herein above. Purified cells were washed in PBS and resuspended in 10 mM HEPES (pH 7.9), 10 mM KCl, 0.1 mM EDTA, 0.1 mM EGTA, 1 mM DTT, and protease inhibitors. Cells were incubated on ice for 15 min. Nonidet P-40 was added to a final concentration of 0.6%, and cells were vortex-mixed vigorously, and the mixture was centrifuged for 5 min. The nuclear pellets were washed twice and resuspended in 20 mM HEPES (pH 7.9), 0.4 M NaCl, 1 mM EDTA, 1 mM EGTA, 1 mM DTT, and protease inhibitors, and the tube was rocked for 30 min at 4° C. After centrifugation for 10 min, the supernatant was collected. Extract proteins (2 μg) were incubated in binding buffer with ³²P-labeled, double-stranded oligonucleotide probe NF-AT, 5′-GCC CAA AGA GGA AAA TTT GTT TCA TAC AG-3′ (SEQ ID NO:17) (Nushift; Active Motif).

### Example 11

### Nr2f6^{-/-} eff/Memory T Cells (Teff) are Partially Resistant to Regulatory T Cell (Treg)-Mediated Immunosuppression

In order to analyse the sensitivity of Nr2f6−/− CD4+ effector T cells, CD4⁺CD25⁺ Treg cells were mixed with CD4⁺CD25⁻ responder T cells (Teff) in different ratios as indicated. As result, we determined that Tregs suppress wild type but not (or at least much less) Nr2f6−/− effector cells in their proliferation or IL-2 and IFNgamma cytokine responses; see FIG. 27. This result is consistent with hyperplasia and late-onset immunopathology of NR2F6^{-/-} mice, indicating an in vivo defect of Treg-mediated homeostasis of CD4+ T cells; see the tables of Example 4. Thus, and again surprisingly, a functional NR2F6 agonist will make T cells hypersensitive to Tregs, while a functional NR2F6 antagonist will make T cells hypersensitive to Treg-mediated immunosuppression.

### Analysis of Proliferation Responses

Antibody- and alloantigen-induced proliferation was described (Pfeifhofer et al., 2006; Pfeifhofer et al., 2003).

### Analysis of Cytokine Production

IL-2 and IFN-γ cytokine amounts from culture supernatant of the CD4⁺ T cells were measured by BioPlex multi-analyte technology (BioRad).

### Example 12

### Spontaneous Tumor Rejection in NR2F6^{-/-} Mice

Surprisingly, gene ablation of NR2F6 has been shown to be sufficient to induce potent anti-tumor immunity in induced mouse cancer models. Tumor xenografts grow significantly slower in NR2F6−/− then in wild-type control mice; see FIG. 28. These results validate that inactivation of NR2F6 as a single negative regulator of effector T cell signalling confers anti-cancer activity in vivo using tumor models relevant for human cancers. Thus, genetic inactivation of NR2F6 appears a suitable strategy for a future anti-cancer adjuvant immunotherapy to augment the effectiveness of tumor specific T cells and to tip the balance of the endogenous immune system towards cancer immune surveillance allowing effective eradication of tumor cells in vivo.

Moreover, siRNA mediated silencing of NR2F6 is effective and leads to hyper-responsiveness of T cells; see FIG. 28. As one example and within the concept of personalized medicine and as a proof of concept, we propose to employ the potentials of (repetitive) adoptive transfer of ex vivo NR2F6 siRNA-silenced T cells to counteract cancer-associated pathological tumor cell immune evasion in combinations with current chemo- and/or immunotherapy regiments and to establish potential additive/synergistic efficacies for long-term amelioration of malignant diseases. Consistently, Nr2f6^{-/-} eff/memory T cells (Teff) are found to be partially resistant to regulatory T cell (Treg)-mediated immunosuppression; see FIG. 28. Together, this discovery thereby provides the unique therapeutically option of directly targeting tumors via endogenous T cell responses in spite of their cancer-associated immunosuppressive mechanisms such as ineffective co-stimulation, impaired CD4⁺ T cell help and/or inhibitory signals by Tregs (Zou et al, Nat Rev Immunol 2006).

In vivo tumor cell growth. EL4 and E.G7 cells were injected s.c. into the shaved left flank of 8-12 weeks old female mice, as indicated. In all experimental groups, tumor growth was monitored three times per week by measuring tumor length, width and height with a caliper. Mice were euthanized when tumor volume reached 1 cm³. All experimental procedures performed on mice were in accordance with institutional guidelines.

### Example 13

### Promoter:Reporter Transrepression Assays for a Cellular NR2F6 Compound Finding Program

NR2F6 transiently overexpressing Jurkat cells show robust and reproducible trans-repression of both CD3/CD28 activation-induced IL-17A (A) as well as a NFAT/AP-1(B) promoter:luciferase reporter; see FIG. 29. As critical controls, NR2F2 wild type, the closest relative of NR2F6, and DNA-binding deficient mutants of NR2F6 such as S83E are not able to suppress CD3/CD28-induced promoter reporter transactivation. Similarly, the C112S zinc finger mutant of NR2F6, established to be defective in DNA binding (data not shown), lost its transcriptional repressor activity. Together this indicates that NR2F6-mediated transcriptional repression is isotype selective and dependent on its DNA binding activity in T cells. This validates an IL17A promoter context dependent cellular HTS in T cells for a NR2F6 compound finding program.

### Reporter Gene Assays

Jurkat-TAg cells (a kind gift from Dr. Crabtree, Stanford University, CA) have been transiently transfected with circular plasmid DNA by electroporation (BTX-T820 ElectroSquarePorator™, ITC, Biotech, Heidelberg, Germany), using predetermined optimal conditions (1×10⁷ cells in 200 μl RPMI medium at 450 V/cm and 5 pulses of 99 msec), yielding approx. 40% transfection efficiency. NF-AT:AP-1 reporter gene expression was measured in transient cotransfection assays using 10 μg pSRα-CD28, 15 μg of the GFP or Nr2f6 expression vectors and 15 μg of the promoter firefly luciferase reporter. The latter were a NF-AT:AP-1 composite site reporter (Macian et al., 2001), a NF-AT reporter construct containing three tandem copies of the NF-AT minimal consensus sequence and the proximal IL-17A promoter reporter (Liu et al., 2004). The proximal IL-17A promoter is also described elsewhere herein. After 21 h cells were stimulated with solid-phase CD3 and CD28 agonistic antibodies for 16 h or left unstimulated, as indicated and the firefly luciferase was measured employing the luciferase detection kit (Promega, Madison, Wis.) and the f3Jet-Luminometer (WALLAC, Turku, Finland).

### Example 14

### Transcriptional Activity of NR2F6 Appears Ligand-Dependent and Thus Drugable

NR2F family members appear as (hydrophobic) ligand-activated receptors whose apparent constitutive activity in cells results from the binding to an endogenous ligand (Kruse et al., PLOS Biol, 2008). LBD of NR2F6 is evolutionary conserved and critical for its transcriptional activity as shown by site-directed mutagenesis of the LBD domain employing the critical Leu 354/355 to Ala double mutant and an AF-2 E383Stop truncation mutant of NR2F6; see FIG. 30. RA served as low-affinity agonist of NR2F6 and was used at 20 microM in this experimental setting. Thus and importantly, the LBD of NR2F6 is critical for the transcriptional repressor activity of NR2F6, indicating that NR2F6-mediated transcriptional repression depends on ligands for the LBD on NR2F6. Again DNA-binding deficient mutants of NR2F6 such as S83E and C112S lost its transcriptional repressor activity. These findings validate NR2F6 as a critical modulator of transcriptional responses in Jurkat T cells and provides strong experimental evidence that it's the LBD of NR2F6 is druggable; Thus NR2F6 is validated as a drug target for immunomodulation therapy.

### Reporter Gene Assays

Jurkat T cells were maintained in RPMI medium supplemented with 10% FCS (Life Technologies, Inc.). Transient transfection of cells was performed by electroporation in a BTX-T820 ElectroSquarePorator (ITC, Biotech, Heidelberg, Germany) apparatus using predetermined optimal conditions: 2×10⁷ cells at 450V/cm and 5 pulses of 99 msec. Optimix medium (Equibio, Kent, UK) was used for studies of promoter reporter gene expression. Reporter gene expression was measured in co-transfection assays using 15 μg of the NR2F6 wild type or mutant expression vectors and 5 μg of the RARE containing renilla luciferase reporter vector pTK-Renilla-Luc (Promega, Medison, Wi). After 24 hrs cells were stimulated with solid phase CD3 and CD28 antibodies with or without retinoic acid (RA at 20 microM) for 16 hrs, as indicated.

1. A composition comprising an antagonist of NR2F6.

2. (canceled)

3. The composition of claim 1 wherein said antagonist of NR2F6 is selected from the group consisting of small binding molecules, RNAi, siRNA, shRNA, stRNA, anti-NR2F6 antisense molecules, intracellular binding-partners, aptamers and intramers.

4. The composition of claim 3, wherein said siRNA molecule is selected from the group consisting of:

| | |
|---|---|
| (a) GUGGAAAGCAUUACGGCGUUU | (SEQ ID No: 19) |
| and | |
| 5′-PACGCCGUAAUGCUUUCCACUU; | (SEQ ID No: 20) |
| (b) AGGUGGAUGCUGCGGAGUAUU | (SEQ ID No: 21) |
| and | |
| 5′-PUACUCCGCAGCAUCCACCUUU; | (SEQ ID No: 22) |
| (c) GCAUCGACAACGUGUGCGAUU | (SEQ ID No: 23) |
| and | |
| 5′-PUCGCACACGUUGUCGAUGCUU; | (SEQ ID No: 24) |
| and | |
| (d) GGCAAGACACCCAUCGAGAUU; | (SEQ ID No: 25) |
| and | |
| 5′-PUCUCGAUGGGUGUCUUGCCUU. | (SEQ ID No: 26) |

5. A method of treating a disease related to an insufficient immune response comprising administering an effective amount of an antagonist of NR2F6, wherein said disease related to an insufficient immune response is primary immunodeficiency or acquired immunodeficiency.

6. The method of claim 5, wherein said acquired immunodeficiency is multiple myeloma, chronic lymphatic leukaemia, drug-induced immunosuppression or acquired immune deficiency syndrome (AIDS).

7. The method of claim 5, wherein said disease related to an insufficient immune response is cancer.

8. The method of claim 7, wherein said cancer is a solid tumor-induced cancer.

9. The method of claim 7, wherein said antagonist of NR2F6 is co-administered with an anti-tumor chemotherapeutic agent, dendritic cell (DC)-based tumor vaccines and/or anti-tumor radiation.

10. The composition of claim 1 further comprising a pharmaceutical carrier.

11. A method for identifying immuno-augmenting agents comprising
(a) contacting a cell, tissue or a non-human animal comprising a reporter construct for NR2F6-inhibition with a candidate molecule;
(b) measuring the reporter signal; and
(c) selecting a candidate molecule which alters the reporter signal.

12. The method of claim 11, wherein said reporter construct for NR2F6-inhibition is selected from the group consisting of a ligand-mediated reporter gene expression construct, a ligand displacement construct, a fluorescent cellular sensor fusion mutant construct, and a ligand-induced homo- and/or heterodimer construct.

13. The method of claim 12, wherein
(a) said ligand-mediated reporter gene expression construct comprises an NR2F6-promoter reporter and consecutively expressed NR2F6, whereby a change in ligand binding to said NR2F6-promoter reporter leads to a change in reporter signal;
(b) said ligand displacement construct comprises ligand-binding domain of NR2F6 and a nuclear receptor-ligand or co-receptor protein, whereby displacement of said nuclear receptor-ligand or co-receptor protein from said ligand-binding domain of NR2F6 leads to a fluorescence polarization (FP) or scintillation proximity (SP) signal;
(c) said fluorescent cellular sensor fusion mutant construct comprises a reporter protein that is fused to a ligand-binding domain of NR2F6, whereby binding of a ligand to said ligand-binding domain of NR2F6 leads to a change in reporter signal; or
(d) said ligand-induced homo- and/or heterodimer construct comprises a NR2F6 or a dimerization-capable part thereof and a dimerization partner of NR2F6, whereby dimerization of said NR2F6 or said part thereof and said dimerization partner of NR2F6 leads to a fluorescence resonance energy transfer (FRET) signal.

14-16. (canceled)

17. The method of claim 13, wherein said dimerization partner of NR2F6 is selected from the group consisting of PPAR, RXR, RAR, VDR, T3R, NF-AT, AP-1 and Nur77.

18. A method for assessing the activity of a candidate antagonist of NR2F6 comprising the steps of:
(a) contacting a cell, tissue or a non-human animal comprising NR2F6 with said candidate;
(b) detecting a decrease in NR2F6 activity or an increase in the activity and/or expression of at least one component of the NR2F6-dependent intracellular signalling cascade; and
(c) selecting a candidate that decreases NR2F6 activity or increases the activity and/or expression of at least one component of the NR2F6-dependent intracellular signalling cascade;
wherein a decrease of the NR2F6 activity or increase of the activity and/or expression of at least one component of the NR2F6-dependent intracellular signalling cascade is indicative for the capacity of the candidate to augment immune response.

19. (canceled)

20. The method of claim 18, wherein said component is selected from the group consisting of NF-AT, AP-1, IL2, IL17 and IFN gamma.

21. The method of claim 11, wherein said cells are Jurkat T-cells.

22. The method of claim 11, wherein said non-human animal is a transgenic non-human animal.

23. A non-human transgenic animal comprising a reporter construct for NR2F6-inhibition or cells or tissue derived therefrom

24. A reporter construct for detection of NR2F6-inhibition comprising:
(a) a ligand-mediated reporter gene expression construct comprising an NR2F6-promoter reporter and consecutively expressed NR2F6, whereby a change in ligand binding to said NR2F6-promoter reporter leads to a change in reporter signal;
(b) a ligand displacement construct comprising a ligand-binding domain of NR2F6 and a nuclear receptor-ligand or co-receptor protein, whereby displacement of said nuclear receptor-ligand or co-receptor protein from said ligand-binding domain of NR2F6 leads to a fluorescence polarization (FP) or scintillation proximity (SP) signal;
(c) a fluorescent cellular sensor fusion mutant construct comprising a reporter protein that is fused to a ligand-binding domain of NR2F6, whereby binding of a ligand to said ligand-binding domain of NR2F6 leads to a change in reporter signal; or
(d) a ligand-induced homo- and/or heterodimer construct comprising a NR2F6 or a dimerization-capable part thereof and a dimerization partner of NR2F6, whereby dimerization of said NR2F6 or said part thereof and said dimerization partner of NR2F6 leads to a fluorescence resonance energy transfer (FRET) signal.

25. (canceled)

26. A kit for assessing the activity of a candidate antagonist of NR2F6 comprising polynucleotides and/or antibodies capable of detecting the activity of NR2F6 or capable of detecting a change in the activity of components of the NR2F6-dependent intracellular signal cascade.

27. (canceled)
